(19) United States
(12) Patent Application Publication
(10) Pub. No.: US 2008/0243049 A1
(43) Pub. Date: Oct. 2, 2008

(54) Biodegradable Triblock Copolymers for Use in Acoustically Mediated Intracellular Drug Delivery in vivo

(75) Inventor: Charles Thomas Hardy, Foster City, CA (US)

Correspondence Address: Biovaluation & Analysis, Inc., 509 Jibstay Lane, Foster City, CA, 94404, US

(73) Assignee: Biovaluation & Analysis, Inc.

(21) Appl. No.: 12/133,631

(22) Filed: Jun. 5, 2008

Related U.S. Application Data
(60) Provisional application No. 60/943,603, filed on Jun. 13, 2007.
(60) Provisional application No. 60/943,589, filed on Jun. 13, 2007.
(60) Provisional application No. 60/943,584, filed on Jun. 13, 2007.
(60) Provisional application No. 60/943,574, filed on Jun. 13, 2007.
(60) Provisional application No. 60/942,453, filed on Jun. 6, 2007.
(60) Provisional application No. 60/942,451, filed on Jun. 6, 2007.
(60) Provisional application No. 60/942,447, filed on Jun. 6, 2007.
(60) Provisional application No. 60/942,443, filed on Jun. 6, 2007.
(60) Provisional application No. 60/942,438, filed on Jun. 6, 2007.

(51) Int. Cl. A61N 7/00; A61M 31/00; A61B 6/12

(52) U.S. Cl. 604/22; 604/59; 600/431

(57) ABSTRACT

Targeted therapeutic delivery systems comprising specially designed nanocarriers for intracellular therapeutic delivery, mediated by acoustic energy, for use either in vivo or in vitro, are described. Nanocarriers comprised of substantially biodegradable triblock copolymers, and mixtures thereof, are used to treat a variety of diseases in humans and other species, such as cancer, opthalmological, pulmonary, urinary or other pathologies. Methods for preparing the targeted therapeutic delivery systems are also embodied, which comprise processing a solution comprised of biopolymers or other species and components, with or without targeting moieties, adding said biopolymers and other compounds to a solution containing one or more therapeutic agents, stabilizing or not stabilizing said nanocarriers, adding one or more contrast agents, and resulting in a targeted therapeutic delivery system. Preferred therapeutics for use with the present invention include nucleic acids, proteins, peptides, and other therapeutic macromolecules.

### CROSS-REFERENCES

The present application claims the benefit of my Provisional Application No. 60/943,603, Methods and Systems for Utilizing Supramolecular Assemblies in Pulsed Cavitation-mediated Ultrasonic Drug Delivery, filed on Jun. 13, 2007; and the benefit of my Provisional Application No. 60/943,589, Methods and Systems for Utilizing Polymersomes and Peptosomes in Pulsed Cavitation-mediated Ultrasonic Drug Delivery, filed on Jun. 13, 2007; and the benefit of my Provisional Application No. 60/943,584, Methods and Systems for Utilizing Dendritic and Branched Chain Polymers in Pulsed Cavitation-mediated Ultrasonic Drug Delivery, filed on Jun. 13, 2007; and the benefit of my Provisional Application No. 60/943,574, Methods and Systems for Utilizing Biodegradable Tri-block Copolymers in Cavitation-mediated Ultrasonic Drug Delivery, filed on Jun. 13, 2007, now abandoned; and the benefit of my Provisional Application No. 60/942,453, Supramolecular assemblies, and Mixtures of the Same, For Acoustically Mediated Intracellular Drug Delivery in vivo, filed on Jun. 6, 2007; and the benefit of my Provisional Application No. 60/942,451, Polymersomes, Peptosomes, and Mixtures of the Same, For, Acoustically Mediated Intracellular Drug Delivery in vivo, filed on Jun. 6, 2007; and the benefit of my Provisional Application No. 60/942,447, Methods and Systems for Pulsed Cavitation-mediated Ultrasonic Drug Delivery, filed on Jun. 6, 2007; and the benefit of my Provisional Application No. 60/942,443, Dendritic and Branched Chain Polymers, and Mixtures of the Same, for Acoustically Mediated Intracellular Drug Delivery in vivo, filed on Jun. 6, 2007, now abandoned; and the benefit of my Provisional Application No. 60/942,438, Biodegradable Tri-block Copolymers, and Mixtures of the Same, for Acoustically mediated Intracellular Drug Delivery in vivo, filed on Jun. 6, 2007; where I am the sole inventor on all applications. All of the aforementioned specifications (i.e., applications) are incorporated herein by reference in their entirety for all purposes.

### REFERENCES CITED

### U.S. Patent Documents

| Patent | Issue Date | Inventor |
|---|---|---|
| 4,162,282 | July 1979 | Fulwyler et al. |
| 4,310,505 | January 1982 | Baldeschwieler et al. |
| 4,410,688 | October 1983 | Denkewalter et al. |
| 4,507,466 | March 1985 | Tomalia et al. |
| 4,533,254 | August 1985 | Cook et al. |
| 4,586,512 | May 1986 | Do-huu et al. |
| 4,620,546 | November 1986 | Aida et al. |
| 4,638,045 | November 1987 | Kohn et al. |
| 4,658,828 | April 1986 | Dory |
| 4,728,575 | March 1988 | Gamble et al. |
| 4,728,578 | March 1988 | Higgins et al. |
| 4,737,323 | April 1988 | Martin et al. |
| 4,806,621 | February 1989 | Kohn et al. |
| 4,863,735 | September 1989 | Kohn et al. |
| 4,921,706 | May 1990 | Roberts et al. |
| 4,980,449 | December 1990 | Kohn et al. |
| 5,099,060 | March 1992 | Kohn et al. |
| 5,120,657 | June 1992 | McCabe et al. |
| 5,128,326 | July 1992 | Balazs et al. |
| 5,140,094 | August 1992 | Kohn et al. |
| 5,194,570 | March 1993 | Kohn et al. |
| 5,196,343 | March 1993 | Zerhouni et al. |
| 5,198,507 | March 1993 | Kohn et al. |
| 5,216,115 | June 1993 | Kohn et al. |
| 5,219,564 | June 1993 | Zalipsky et al. |
| 5,242,997 | September 1993 | Kohn et al. |
| 5,264,537 | November 1993 | Kohn et al. |
| 5,312,617 | May 1994 | Unger et al. |
| 5,317,077 | May 1994 | Kohn et al. |
| 5,455,027 | October 1995 | Zalipsky et al. |
| 5,587,507 | December 1996 | Kohn et al. |
| 5,658,995 | August 1997 | Kohn et al. |
| 5,670,602 | September 1997 | Kohn et al. |
| 5,580,960 | December 1996 | Burgeson et al. |
| 5,610,031 | March 1997 | Burgeson et al. |
| 5,625,040 | April 1997 | Margolis et al. |
| 5,648,465 | July 1997 | Margolis et al. |
| 5,728,379 | March 1998 | Martuza et al. |
| 5,766,922 | June 1998 | Peles |
| 5,770,222 | June 1998 | Unger et al. |
| 5,770,565 | June 1998 | Cheng et al. |
| 5,792,743 | August 1998 | Schachner |
| 5,836,905 | November 1998 | Lemelson et al. |
| 5,849,865 | December 1998 | Cheng et al. |
| 5,866,165 | February 1999 | Liu et al. |
| 5,872,231 | February 1999 | Engvall et al. |
| 5,877,224 | March 1999 | Kohn et al. |
| 5,935,553 | August 1999 | Unger et al. |
| 6,048,521 | April 2000 | Kohn et al. |
| 6,063,629 | May 2000 | Knoblauch |
| 6,517,824 | February 2003 | Kohn et al. |
| 6,602,497 | August 2003 | Kohn et al. |
| 6,852,308 | February 2005 | Kohn et al. |
| 7,005,454 | February 2006 | Brocchini et al. |
| 6,071,495 | June 2000 | Unger et al. |
| 6,103,255 | August 2000 | Levene et al. |
| 6,113,946 | September 2000 | Szoka et al. |
| 6,120,491 | September 2000 | Kohn et al. |
| 6,121,231 | September 2000 | Petit et al. |
| 6,139,819 | October 2000 | Unger et al. |
| 6,140,117 | October 2000 | Milbrandt et al. |
| 6,146,657 | November 2000 | Unger et al. |
| 6,159,467 | December 2000 | Chung et al. |
| 6,169,078 | January 2001 | Hughes et al. |
| 6,200,547 | March 2001 | Volkonsky et al. |
| 6,204,054 | March 2001 | Sutton et al. |
| 6,267,987 | July 2001 | Park et al. |
| 6,284,862 | September 2001 | Kohn et al. |
| 6,319,492 | November 2001 | Kohn et al. |
| 6,319,703 | November 2001 | Speck |
| 6,331,658 | December 2001 | Cooper et al. |
| 6,337,198 | January 2002 | Levene et al. |
| 6,335,011 | January 2002 | Podsakoff et al. |
| 6,344,446 | February 2002 | Gorman et al. |
| 6,352,972 | March 2002 | Nimni et al. |
| 6,359,102 | March 2002 | Kemnitzer et al. |
| 6,372,720 | April 2002 | Longmuir et al. |
| 6,383,500 | May 2002 | Wooley et al. |
| 6,399,663 | June 2002 | Haces et al. |
| 6,403,056 | June 2002 | Unger |
| 6,416,740 | July 2002 | Unger |
| 6,436,709 | August 2002 | Lin et al. |
| 6,461,641 | October 2002 | Fick |
| 6,475,477 | November 2002 | Kohn et al. |
| 6,482,410 | November 2002 | Crossin |
| 6,482,436 | November 2002 | Volkonsky et al. |
| 6,488,615 | December 2002 | Mitchiner et al. |
| 6,514,762 | February 2003 | Wang |
| 6,548,740 | April 2003 | Bremel et al. |
| 6,593,130 | July 2003 | Sen et al. |
| 6,603,998 | August 2003 | King et al. |
| 6,610,290 | August 2003 | Podsakoff et al. |
| 6,616,944 | September 2003 | Kissel et al. |
| 6,632,671 | October 2003 | Unger |
| 6,663,555 | December 2003 | Mitchiner et al. |
| 6,716,882 | April 2004 | Haces et al. |
| 6,746,860 | June 2004 | Tokusumi et al. |
| 6,852,308 | February 2005 | Kohn et al. |
| 6,773,696 | August 2004 | Unger |
| 6,780,846 | August 2004 | O'Mahony et al. |
| 6,872,406 | March 2005 | Qi |
| 6,878,541 | April 2005 | Qiao et al. |
| 6,978,172 | December 2005 | Mori et al. |
| 6,998,107 | February 2006 | Unger |
| 7,001,614 | February 2006 | Smyth-Templeton et al. |
| 7,083,572 | August 2006 | Unger et al. |
| 7,081,495 | July 2006 | Florence et al. |
| 7,087,729 | August 2006 | Prive |
| 7,089,053 | August 2006 | Tokudome et al. |
| 7,115,380 | October 2006 | Allinquant |
| 7,127,284 | October 2006 | Seward |
| 7,138,562 | November 2006 | Bremel et al. |
| 7,153,905 | December 2006 | Banerjee et al. |
| 7,195,780 | March 2007 | Dennis et al. |
| 7,241,617 | July 2007 | Fukumura et al. |
| 7,250,154 | July 2007 | Kohn et al. |
| 7,271,234 | September 2007 | Kohn et al. |
| 7,268,214 | September 2007 | O'Mahony et al. |
| 7,271,234 | September 2007 | Kohn et al. |
| 7,288,266 | October 2007 | Smyth-Templeton et al. |
| 7,297,838 | November 2007 | Chen et al. |
| 7,309,757 | December 2007 | Liu et al. |
| 7,314,614 | January 2008 | Yonemitsu et al. |
| 7,345,138 | March 2008 | Wang et al. |

### REFERENCES CITED

### U.S. Patent Applications

| Application | Submission Date | Inventor |
|---|---|---|
| 11/177,827 | July 2005 | Fedewa et al. |
| 10/514,215 | May 2003 | Kohn et al. |
| 10/952,274 | September 2004 | Brandom et al. |
| 11/001,250 | December 2004 | Kohn et al. |
| 11/191,181 | July 2005 | Kohn et al. |
| 10/952,202 | February 2006 | Kohn et al. |

### REFERENCES CITED

### Other References

Abragam, A., Principles of Nuclear Magnetism (International Series of Monographs on Physics), University Press, 1983, Oxford, England.
Abramson et al., “A Computational Approach to Predicting Cell Growth on Polymeric Biomaterials,” J. Biomed. Mater. Res. A., 2005, 73(1), 116.
Akhtar et al., “Nonviral Delivery of Synthetic siRNAs in vivo,” J. Clin. Invest., 2007, 117(12), 3623-3632.
Al-Jamal et al., “Supramolecular Structures from Dendrons and Dendrimers,” Adv. Drug Deliv. Rev., 2005, 57(15), 2238.
Anderson, J. M. et al., “Poly-Alpha Amino Acids as Biomedical Polymers,” Biocompatibility of Tissue Analogs, Vol. 1., pp. 67-88, D. E Williams, CRC Press, 1985, Boca Raton, Fla.
Auguste et al., “Association of Hydrophobically-Modified Poly(Ethylene Glycol) with Fusogenic Liposomes,” Biochim. Biophys. ACTA, 2003, 1616(2), 184.
Auguste et al., “pH Triggered Release of Protective Poly(Ethylene Glycol)-B-Polycation Copolymers from Liposomes,” Biomaterials, 2006, 27(12), 2599.
Baban et al., “Control of Tumour Vascular Permeability,” Adv. Drug. Deliv. Rev., 1998, 34(1), 109-119.
Bae et al., “Minute Changes in Composition of Polymer Substrates Produce Amplified Differences in Cell Adhesion and Motility Via Optimal Ligand Conditioning,” ACTA Biomater., 2006, 2(5), 473.
Bailey et al., “Cellular Response to Phase-Separated Blends of Tyrosine-Derived Polycarbonates,” J. Biomed. Mater. Res. A., 2006, 76(3), 491.
Basu et al., Liposome Methods and Protocols, Totowa, Humana Press, 2002, New Jersey.
Bhaskar et al., “Effect of an Applied Electric Field on Liquid Crystalline Membranes: Control of Permeability,” J. Membr. Sci., 1985, 1, 83.
Bishop, P., “The Biochemical Structure of Mammalian Vitreous,” Eye, 1996, 10, 664-670.
Bland et al., Ultrathin Magnetic Structures I: An Introduction to the Electronic Magnetic and Structural Properties, Springer, 1994, Berlin, Germany; New York, N. Y.
Bourke et al., “A Photo-Crosslinked Poly(Vinyl Alcohol) Hydrogel Growth Factor Release Vehicle for Wound Healing Applications,” AAPS Pharm. Sci., 2003a, 5(4), E33.
Bourke et al., “Polymers Derived from the Amino Acid L-Tyrosine: Polycarbonates, Polyarylates and Copolymers with Poly(Ethylene Glycol),” Adv. Drug Deliv. Rev., 2003b, 55(4), 447.
Bourke et al., “Preliminary Development of a Novel Resorbable Synthetic Polymer Fiber Scaffold for Anterior Cruciate Ligament Reconstruction,” Tissue Eng., 2004, 10(1-2), 43.
Brocchini et al., “Structure-Property Correlations in a Combinatorial Library of Degradable Biomaterials,” J. Biomed. Mater. Res., 1998, 42(1), 66.
Burov, A., “High-Intensity Ultrasonic Vibrations for Action on Animal and Human Malignant Tumours,” Dokl. Akad. Nauk., SSSR, 1956, 106, 239-241.
Capecci, M., “High Efficiency Transformation by Direct Microinjection of DNA into Cultured Mammalian Cells,” Cell, 1980, 22(2 Pt 2), 479-88.
Carson et al., “Ultrasonic Power and Intensities Produced by Diagnostic Ultrasound Equipment,” Ultrasound Med. Biol., 1978, 3(4), 341.
Chaikof et al., “Bioscaffolds for Tissue Repair: Breakout Session Summary,”, Ann. N.Y. Acad. Sci., 2002a, 961, 112.
Chaikof et al., “Biomaterials and Scaffolds in Reparative Medicine,” Ann. N.Y. Acad. Sci., 2002b, 961, 96.
Chen et al., “Magnetically Responsive Polymerized Liposomes as Potential Oral Delivery Vehicles,” Pharm. Res., 1997, 14(4), 537.
Choueka et al., “Canine Bone Response to Tyrosine-Derived Polycarbonates and Poly(L-Lactic Acid),” J. Biomed. Mater. Res., 1996, 31(1), 35.
Colowick et al., Drug and Enzyme Targeting, Part A: Volume 112: Drug and Enzyme Targeting (Methods in Enzymology), Academic Press, 2006, New York, N.Y.
Craik, D., Magnetism: Principles and Applications, Wiley, 1995, Chichester, N.Y.
Duvvuri et al., “Drug Delivery to the Fetina: Challenges and Opportunities,” Expert. Opin. Biol. Ther., 2003, 3, 45-56.
Duzgunes, N., Liposomes, Part A, Volume 367 (Methods in Enzymology), Academic Press, 2003a, New York, N.Y.
Duzgunes, N., Liposomes, Part C, Volume 373 (Methods in Enzymology), Academic Press, 2003b, New York, N.Y.
Duzgunes, N., Liposomes, Part D, Volume 387 (Methods in Enzymology), Academic Press, 2004, New York, N.Y.
Duzgunes, N., Liposomes, Part E, Volume 391 (Methods in Enzymology), Academic Press, 2005, New York, N. Y.
Engelberg et al., “Physico-Mechanical Properties of Degradable Polymers Used in Medical Applications: A Comparative Study,” Biomaterials, 1991, 12(3), 292.
Ertel et al., “Evaluation of a Series of Tyrosine-Derived Polycarbonates as Degradable Biomaterials,” J. Biomed. Mater. Res., 1994, 28(8), 919.
Ertel et al., “Evaluation of Poly(Dth Carbonate), a Tyrosine-Derived Degradable Polymer, for Orthopedic Applications,” J. Biomed. Mater. Res., 1995, 29(11), 1337.
Fellinger et al., “Klinik und Therapies des Chemischen Gelenkreumatismus,” Maudrich Vienna, Austria, 1954, 549-552.
Fiordeliso et al., “Design, Synthesis, and Preliminary Characterization of Tyrosine-Containing Polyarylates: New Biomaterials for Medical Applications,” J. Biomater. Sci. Polym. Ed., 1994, 5(6), 497.
Fouassier, J., Photoinitiation, Photopolymerization, and Photocuring: Fundamentals and Applications, Distributed by Hanser/Gardner Publications, Hanser, 1995, Munich, Germany; New York, N.Y.; Cincinnati, Ohio.
Golemis et al., Protein-Protein Interactions: A Molecular Cloning Manual, Cold Spring Harbor Laboratory Press, 2005, Cold Spring Harbor, N. Y.
Greco et al., “Polymer of Proline Analogue with Sustained Antifibrotic Activity in Lung Fibrosis,”Am. J. Respir. Crit. Care Med., 1997, 155(4), 1391.
Gregoriadis, G., Liposome Technology, Third Edition, Informa Healthcare, 2006, New York, N. Y.
Gennaro et al., Remington: The Science and Practice of Pharmacy, 19th edition, Mack Publishing Company, 1995.
Guzmán et al., “Ultrasound-mediated Disruption of Cell Membranes. I. Quantification of Molecular Uptake and Cell Viability,” J. Acoust. Soc. Am., 2001a, 110(1): 588-596.
Guzmán et al., “Ultrasound-mediated Disruption of Cell Membranes. II. Heterogeneous Effects on Cells,” J. Acoust. Soc. Sm., 2001b, 110(1), 597-606.
Guzmán et al., “Equilibrium loading of Cells with Macromolecules by Ultrasound: Effects of Molecular Size and Acoustic Energy,” J. Pharm. Sci., 2002, 91(7), 1693-1701.
Guzmán et al., “Bioeffects Caused by Changes in Acoustic Cavitation Bubble Density and Cell Concentration: A Unified Explanation Based on Cell-to-Bubble Ratio and Blast Radius,” Ultrasound Med. Biol., 2003, 29(8), 1211-1222.
Harlow et al., Using Antibodies: A Laboratory Manual, Cold Spring Harbor Laboratory Press, 1999, Cold Spring Harbor, N. Y.
He et al., “Studies on Pharmacologic Disposition of Adriamycin in Dog after Hepatic Arterial Embolization with Adriamycin Carboxymethyl-Dextran-Microspheres,” Yao Xue Xue Bao, 1993, 28(11), 859.
Hench et al., Biomaterials—an Interfacial Approach, Biophysics and Bioengineering Series. A, Academic Press, 1982, Noordergraaf, Germany; New York, N.Y.,
Hermanson, G., Bioconjugate Techniques, Academic Press, 1996, San Diego, Calif.
Hille, B., Ion Channels of Excitable Membranes, Sinauer Associates, Inc., 2001, Sunderland, Mass.
Hooper et al., “Comparative Histological Evaluation of New Tyrosine-Derived Polymers and Poly (L-Lactic Acid) as a Function of Polymer Degradation,” J. Biomed. Mater. Res., 1998, 41(3), 443.
Hooper et al., “Characterization of the Inflammatory Response to Biomaterials Using a Rodent Air Pouch Model,” J. Biomed. Mater. Res., 2000, 50(3), 365.
Hsieh et al., “Magnetic Modulation of Release of Macromolecules from Polymers,” Proc. Natl. Acad. Sci. U.S.A., 1981, 78(3), 1863.
James et al., “Small Changes in Polymer Chemistry Have a Large Effect on the Bone-Implant Interface: Evaluation of a Series of Degradable Tyrosine-Derived Polycarbonates in Bone Defects,” Biomaterials, 1999, 20(23-24), 2203.
Jones et al., “Experimental Examination of a Targeted Hyperthermia System Using Inductively Heated Ferromagnetic Microspheres in Rabbit Kidney,” Phys. Med. Biol., 2001, 46(2), 385.
Kohn et al., “Single-Step Immunization Using a Controlled Release, Biodegradable Polymer with Sustained Adjuvant Activity,” J. Immunol. Methods, 1986a, 95(1), 31.
Kohn et al., “Poly(Iminocarbonates) as Potential Biomaterials.” Biomaterials, 1986b, 7(3), 176.
Kohn et al., “Identification and Colorimetric Determination of Organic Cyanates in Nanomolar Quantities,” Anal. Chem., 1986c, 58(14), 3184.
Kohn et al., “Polymerization Reactions Involving the Side Chains of A-L-Amino Acids,” J. Am. Chem. Soc., 1987, 109, 817.
Kohn et al., “Backbone Modifications of Synthetic Poly-A-L-Amino Acids,” In: Peptides—Chemistry and Biology: Proceedings of the 10th American Peptide Symposium, Marshall et al., Leiden Escom Publishing, 1988, 658-661.
Kohn, J., “Biomaterials Science at a Crossroads: Are Current Product Liability Laws in the United States Hampering Innovation and the Development of Safer Medical Implants?” Pharm. Res. 1996, 13(6), 815.
Kohn, J., “Current Trends in the Development of Synthetic Materials for Medical Applications,” Med. Device Technol., 1990, 1(6), 34.
Kohn, J., “Pseudo-Poly(Amino Acids),” Drug News Perspect., 1991a, 4(5), 289.
Kohn, J., “The Use of Natural Metabolites in the Design of Non-Toxic Polymers for Medical Applications,” Polym. News, 1991b, 16(11), 325.
Kohn, J., “Desaminotyrosyl-Tyrosine Alkyl Esters: New Diphenolic Monomers for the Design of Tyrosine-Derived Pseudopoly(Amino Acids),” In: Polymeric Drugs and Drug Delivery Systems, Acs Symposium, Series Vol. 469., Dunn et al., American Chemical Society, 1991c, Washington, D.C., 155-169.
Kohn et al., “Engineering the Future,” Rehab. Manag., 1994, 7(1), 30.
Kong et al., “Hyperthermia Enables Tumor-specific Nanoparticle Delivery: Effect of Particle Size,” Cancer Res., 2000, 60, 4440-4445.
Kohn, J., “New Approaches to Biomaterials Design,” Nat. Mater., 2004a, 3 (11), 745.
Kohn et al., “Random-Coil Behavior and the Dimensions of Chemically Unfolded Proteins.” Proc. Natl. Acad. Sci. U.S.A, 2004b, 101(34), 12491.
Kohn et al., “Degradable, Drug-Eluting Stents: A New Frontier for the Treatment of Coronary Artery Disease,” Expert Rev. Med. Devices, 2005a, 2(6), 667.
Kohn et al., “Engineering a Signal Transduction Mechanism for Protein-Based Biosensors,” Proc. Natl. Acad. Sci. U.S.A., 2005b, 102(31), 10841.
Kost et al., “Magnetically Enhanced Insulin Release in Diabetic Rats,” J. Biomed. Mater. Res., 1987, 21 (12), 1367.
Lamba et al., Polyurethanes in Biomedical Applications, CRC Press, 1998, Boca Raton, Fla.
Laskey, R., “CIBA Medal Lecture, Regulatory Roles of the Nuclear Membrane,” Biochem. Soc. Trans., 1998, 26(4), 561-567.
Lechardeur et al., “Determinants of the Nuclear Localization of the Heterodimeric DNA Fragmentation Factor (ICAD/CAD)” J. Cell Biol., 2000, 150(2), 321-334.
Lescure et al., J. Biomed. Mat. Res., 1989, 23, 1299.
Liu et al., “Quantitative Biorelevant Profiling of Material Microstructure within 3d Porous Scaffolds Via Multiphoton Fluorescence Microscopy,” J. Biomed. Mater. Res. B. Appl. Biomater., 2007.
Luby-Phelps, K., “Cytoarchitecture and Physical Properties of Cytoplasm: Volume, Viscosity, Diffusion, Intracellular Surface Area,” Int. Rev. Cytol., 2000, 192, 189-221.
Lu et al., Cellular Drug Delivery: Principles and Practice, Humana Press, 2004, Totowa, N.J.
Lubbe et al., “Clinical Applications of Magnetic Drug Targeting,” J. Surg. Res., 2001, 95 (2), 200.
Lyman, D., Polymers in Medicine—an Overview, Polymers in Medicine. E. Chiellini et al., Plenum Press, 1982, New York, 215-218.
Mahato, R., Biomaterials for Delivery and Targeting of Proteins and Nucleic Acids, CRC Press, 2005, Boca Raton, Fla.
McNeil et al., “Plasma Membrane Disruption: Repair, Prevention, Adaptation,” Annu. Rev. Cell. Dev. Biol., 2003, 19, 697-731.
Meechaisue et al., “Electrospun Mat of Tyrosine-Derived Polycarbonate Fibers for Potential Use as Tissue Scaffolding Material,” J. Biomater. Sci. Polym. Ed., 2006, 17(9), 1039.
Miller et al., Magnetism: Molecules to Materials, Wiley-VCH, 2001, Weinheim, N.Y.
Molema et al., Drug Targeting: Organ-Specific Strategies, Wiley-VCH, 2001, Weinheim, N.Y.
Moroz et al., “Targeting Liver Tumors with Hyperthermia: Ferromagnetic Embolization in a Rabbit Liver Tumor Model,” J. Surg Oncol., 2001, 78(1), 22.
Muzykantov et al., Biomedical Aspects of Drug Targeting, Kluwer Academic Publishers, 2002, Boston, Mass.
Nardin et al., “Nontoxic Block Copolymer Nanospheres: Design and Characterization,” Langmuir, 2004, 20(26), 11721.
Nathan et al., “Copolymers of Lysine and Polyethylene Glycol: A New Family of Functionalized Drug Carriers,” Bioconjug. Chem., 1993, 4(1), 54.
Neumann et al., “Gene Transfer into Mouse Lyoma Cells by Electroporation in High Electric Fields,” Embo. J. 1982, 1(7), 841-845.
Nijenhuis et al., Chemical and Physical Networks: Formation and Control of Properties, Wiley, 1998, Chichester, N.Y.
Nyborg et al., “Biological Effects of Ultrasound: Development of Safety Guidelines. Part II: General Review,” Ultrasound Med. Biol., 2001, 27(3), 301-333.
O'Brien, W., “Ultrasound-biophysics Mechanisms,” Prog. Biophys. Mol. Biol., 2007, 93, 212-255.
Ottenbrite et al., Polymeric Drugs & Drug Delivery Systems, Technomic Pub., 2001, Lancaster, Pa.
Parang et al., “Development of Photo-Crosslinking Reagents for Protein Kinase-Substrate Interactions,” FEBS Lett., 2002, 520(1-3), 156.
Parkinson et al., “Crystal Structure and NMR Conformation of a Cyclic Pseudotetrapeptide Containing Urethane Backbone Linkages,” Biopolymers, 1994, 34(3), 403.
Peeters et al., “Vitreous: a Barrier to Nonviral Ocular Gene Therapy,”, Invest. Opthalmol. Vis. Sci., 2005, 46, 3553-3561.
Pitkanen et al., “Vitreous is a Barrier in Nonviral Gene Transfer by Cationic Lipids and Polymers,” Pharm. Res., 2003, 20, 576-583.
Poiani et al., “Polymeric Carrier of Proline Analogue with Antifibrotic Effect in Pulmonary Vascular Remodeling,” Am. J. Respir. Crit. Care Med., 1997, 155(4), 1384.
Pulapura et al., “Structure-Property Relationships for the Design of Polyiminocarbonates,” Biomaterials, 1990, 11(9), 666.
Pulapura et al., “Trends in the Development of Bioresorbable Polymers for Medical Applications,” Biomater. Appl., 1992a, 6(3), 216.
Pulapura et al., “Tyrosine-Derived Polycarbonates: Backbone-Modified ‘Pseudo’-Poly (Amino Acids) Designed for Biomedical Applications,” Biopolymers, 1992b, 32(4), 411.
Raspe et al., “S-Allele Diversity in Sorbus Aucuparia and Crataegus Monogyna (Rosaceae: Maloideae),” Heredity, 2002, 88(6), 458.
Rudge et al., “Preparation, Characterization, and Performance of Magnetic Iron-Carbon Composite Microparticles for Chemotherapy,” Biomaterials, 2000, 21(14), 1411.
Ruponen et al., “Extracellular Glycosaminoglycans Modify Cellular Trafficking of Lipoplexes and Polyplexes,” J. Biol. Chem., 2001, 276, 33875-33880.
Sambrook et al., Molecular Cloning: Laboratory Manual, Cold Spring Harbor Laboratory Press, 2001, Cold Spring Harbor, N. Y.
Schachter et al., “A Synthetic Polymer Matrix for the Delayed or Pulsatile Release of Water-Soluble Peptides,” J. Control Release, 2002, 78(1-3), 143.
Schlicher et al., “Mechanism of Intracellular Delivery by Acoustic Cavitation,” Ultrasound Med. Biol., 2006, 32(6), 915-924.
Schreier, H., Drug Targeting Technology: Physical Chemical, Biological Methods, Marcel Dekker, 2001, New York.
Sharma et al., “Poly(Ethylene Glycol) Enhances Cell Motility on Protein-Based Poly(Ethylene Glycol)-Polycarbonate Substrates: A Mechanism for Cell-Guided Ligand Remodeling,” J. Biomed. Mater. Res. A., 2004, 69(1), 114.
Sheihet et al., “Effect of Tyrosine-Derived Triblock Copolymer Compositions on Nanosphere Self-Assembly and Drug Delivery,”, Biomacromolecules, 2007.
Sheihet et al., “Hydrophobic Drug Delivery by Self-Assembling Triblock Copolymer-Derived Nanospheres,” Biomacromolecules, 2005, 6(5), 2726.
Schnell, H., Chemistry and Physics of Polycarbonates, Interscience Publishers, 1964, New York, N.Y.
Sidman et al., “Biodegradable Implantable Sustained Release Systems Based on Glutamic Acid Copolymers,” J. Membr. Sci., 1980, 7, 277.
Sidman et al., “Controlled Release of Macromolecules and Pharmaceuticals from Synthetic Polypeptides Based on Glutamic Acid,” Biopolymers, 1983, 22, 547.
Silver et al., “Tissue Compatibility of Tyrosine-Derived Polycarbonates and Polyiminocarbonates: An Initial Evaluation,” J. Long Term Eff. Med. Implants, 1992, 1 (4), 329.
Simionescu, N., “Cellular Aspects of Trans-capillary Exchange,” Physiol. Rev., 1983, 63, 1536-1579.
Singer et al., “The Fluid Mosaic Model of the Structure of Cell Membranes,” Science, 1972, 175(23), 720.
Smith et al., “Using Surrogate Modeling in the Prediction of Fibrinogen Adsorption onto Polymer Surfaces,” J. Chem. Inf. Comput. Sci., 2004, 44(3), 1088.
Sousa et al., “Selective Protein Adsorption on a Phase-Separated Solvent-Cast Polymer Blend,” Langmuir, 2006, 22(14), 6286.
Suarez et al., “The Study of Water Uptake in Degradable Polymers by Thermally Stimulated Depolarization Currents,” Biomaterials, 1998, 19(24), 2347.
Takakura et al., “Extravasation of Macromolecules,” Adv. Drug Deliver. Rev., 1998, 34, 93-108.
Talcott et al., “Getting Across the Nuclear Pore Complex,” Trends Cell. Biol., 1999, 9(8), 312-318.
Tangpasuthadol et al., “Thermal Properties and Physical Ageing Behaviour of Tyrosine-Derived Polycarbonates,” Biomaterials, 1996, 17(4), 463.
Tangpasuthadol et al., “Hydrolytic Degradation of Tyrosine-Derived Polycarbonates, a Class of New Biomaterials. Part I: Study of Model Compounds,” Biomaterials, 2000a, 21(23), 2371.
Tangpasuthadol et al., “Hydrolytic Degradation of Tyrosine-Derived Polycarbonates, a Class of New Biomaterials. Part II: 3-Yr Study of Polymeric Devices,” Biomaterials, 2000b, 21(23), 2379.
Taylor et al., “Differing Hepatic Lesions Caused by the Same Dose of Ultrasound,” J. Pathol., 1969, 98, 291-293.
Taylor et al., “A Randomized Clinical Trial of the Effectiveness of an Acupressure Device (Relief Brief) for Managing Symptoms of Dysmenorrhea,” J. Altern. Complement. Med., 2002, 8(3), 357.
Tønnesen, H., Photostability of Drugs and Drug Formulations, CRC Press, 2004, Boca Raton, Fla.
Torchilin et al., Liposomes: A Practical Approach, Oxford University Press, 2003, Oxford, England; New York, N.Y.
Uchegbu et al., Polymers in Drug Delivery, CRC/Taylor & Francis, 2006, Boca Raton, Fla.
Villanueva et al., “Microbubbles Targeted to Intercellular Adhesion Molecule-1 Bind to Activated Coronary Artery Endothelial Cells,”Circulation, 1998, 98(1), 1.
Wang et al., Drug Delivery: Principles and Applications, Wiley-Interscience, 2005, Hoboken, N.J.
Weber et al., “Small Changes in the Polymer Structure Influence the Adsorption Behavior of Fibrinogen on Polymer Surfaces: Validation of a New Rapid Screening Technique,” J. Biomed. Mater. Res. A, 2004, 68(3), 496.
Weber et al., “Formation of Viscoelastic Protein Layers on Polymeric Surfaces Relevant to Platelet Adhesion,” J. Biomed. Mater. Res. A, 2005, 72(4), 420.
Weber et al., “Viscoelastic Properties of Fibrinogen Adsorbed to the Surface of Biomaterials Used in Blood-Contacting Medical Devices,” Langmuir, 2007, 23(6), 3298.
Weller et al., “Modulating Targeted Adhesion of an Ultrasound Contrast Agent to Dysfunctional Endothelium” Ann. Biomed. Eng, 2002, 30(8), 1012.
Widder et al., “Magnetic Microspheres: Synthesis of a Novel Parenteral Drug Carrier,” J. Pharm. Sci., 1979, 68(1), 79.
Wong et al., “Electric Field Mediated Gene Transfer,” Biochem. Biophys. Res. Commun., 1982, 107(2), 584-7.
Wu et al., “Studies on Distribution of Magnetic Gelatin Microspheres in Rabbits,” Yao Xue Xue Bao, 1993, 28(6), 464.
Wu et al., “Studies on Magnetic Responsiveness and Renal Embolization with Magnetic Microspheres in Dogs,” Yao Xue Xue Bao, 1994, 29(4), 311.
Yu et al., “Tyrosine-Peg-Derived Poly(Ether Carbonate)S as New Biomaterials. Part I: Synthesis and Evaluation,” Biomaterials, 1999a, 20(3), 253.
Yu et al., “Tyrosine-Peg-Derived Poly(Ether Carbonate)S as New Biomaterials. Part II: Study of Inverse Temperature Transitions,” Biomaterials, 1999b, 20(3), 265.
Yuan et al., “Vascular-permeability in a Human Tumor Xenograft—Molecular-size Dependence and Cutoff Size,” Cancer Res., 1995, 55, 3752-3756.

### BACKGROUND

Today, clinical medicine has a long list of diverse pharmaceutical products: every year many new drugs are added to the index. Physicians and patients are never satisfied with just a favorable drug action against the malady under treatment. Rather, the task of avoiding undesirable drug actions on normal organs and tissues, and minimizing side effects is of increasing importance to the global healthcare system. In fact, many pharmacologically effective compounds cannot be used as drugs due to their undesirable action on normal tissues. Further, because of researchers' increasing understanding of the human body and the explosion of new and potential treatments resulting from discoveries of bioactive molecules and gene therapies, pharmaceutical research hangs on the verge of yet another great series of advancements. However, this next leap requires not only the development of new treatments, but also the mechanisms to target and efficiently deliver them.

The rapid developments in biotechnology and molecular biology have made it possible to produce a large number of exciting and novel therapeutics in quantities sufficient enough for large-scale clinical use. Pharmaceutically active peptides and proteins can now be used in the treatment of life-threatening diseases (e.g., cancer and diabetes) and of several types of viral, bacterial, and parasitic diseases, as well as, for example, in vaccines for prophylactic purposes. Nucleic acid-based therapeutics, including plasmids containing transgenes for gene therapy, oligonucleotides for antisense and antigene applications, DNAzymes, aptamers, and small interfering RNAs (siRNA), represent an especially promising class of drugs for the treatment of a wide range of diseases. These include, for example, cancer, AIDS, neurological disorders (e.g., Parkinson's and Alzheimer's disease) as well as cardiovascular disorders. The specialized biological activities of these types of novel therapeutics, which hereafter may be referred to as therapeutic macromolecules, provide tremendous advantages over other types of pharmaceuticals. However, most of these macromolecules require delivery to a well-defined compartment of the body for therapeutic effectiveness, and conventional drug delivery technologies are still largely ineffective at meeting these and other challenges.

Elucidation of the human genome has generated a major impetus in identifying human genes implicated in diseases, which should ultimately lead to the development of therapeutic macromolecules for applications such as, for example, gene replacement, potential targets for gene ablation, and the like. In addition, using genomic data, potent nucleic acid drugs may be developed for individualized medicine. Data from the Human Genome Project will continue to assist in determining genetic markers responsible for patient responses to drug therapy, drug interactions, and potential side effects. Developments in human genomics, transcriptomics, and proteomics will provide an additional impetus for the advancement of nucleic acid-based therapeutic macromolecules by supplying novel targets for drug design, screening, and selection.

Unfortunately, developing adequate delivery systems for most of these new drugs remains one of the major challenges in recognizing the full therapeutic potential of many, and probably the most valuable of these therapeutic macromolecules. Indeed, the innate ability of nucleic acid-based drugs to be internalized by target cells is minimal under normal circumstances. Presently, nucleic acid delivery systems are categorized into four broad categories: (1) mechanical transfection, (2) electrical techniques, (3) chemical methods, and (4) vector-assisted delivery systems. Attempts to deliver therapeutic nucleic acids known in the art employing each of these broad strategies, many for use ultimately in clinical settings, will be briefly reviewed.

Mechanical techniques include the direct injection of nucleic acids (i.e., uncomplexed DNA) into a cell nucleus, which is perhaps the most conceptually simple and appealing approach to gene delivery. Obviously, a major drawback to this method is that microinjection can be achieved only one cell at a time, which limits its approach to individual cell manipulation, such as producing transgenic animals. Information relevant to attempts to address these problems using microinjection can be found in, for example, U.S. Pat. Nos. 6,063,629; 6,331,658; 6,548,740; and 7,138,562. Though relatively efficient, the method is also slow and laborious, and therefore neither appropriate for clinical applications nor in research situations with large numbers of cells. Another mechanical technique is particle bombardment, also called biolistic particle delivery. In this strategy, nucleic acids are introduced into several cells simultaneously by coating, for example, microparticles composed of metals such as gold or tungsten with nucleic acids; the coated particles are then accelerated to high velocity to penetrate cell membranes or cell walls. This is accomplished by an apparatus such as, for example, a ‘gene gun’ (e.g., see U.S. Pat. No. 6,436,709). Information relevant to attempts to introduce therapeutic nucleic acids using particle bombardment can be found in, for example, U.S. Pat. Nos. 5,120,657; 5,836,905; 6,436,709; and 7,297,838. Because of the difficulty in controlling the DNA entry pathway, limited tissue penetration depth, when applied externally, target specificity, as well as many other limitations, this approach has been used primarily in research applications.

Another approach for introducing nucleic acids includes the use of high-voltage electrical pulses to transiently permeabilize cell membranes, thus permitting cellular uptake of macromolecules. This process, called electroporation, was first used to deliver DNA to mammalian cells in 1982 (Neumann et al., 1982; Wong et al., 1982), and subsequently has been used to deliver nucleic acids to a large variety of cells in the laboratory. Information relevant to attempts to ultimately deliver nucleic acids in a clinical environment using electroporation can be found in, for example, U.S. Pat. Nos. 6,514,762; 6,593,130; 6,603,998; 6,978,172; 7,089,053; and 7,127,284. Although useful in the research settings, each one of these preceding applications is seriously limited because of, for example, the high mortality of cells after high-voltage exposure and difficulties in optimization.

The use of uptake-enhancing chemicals, which is arguably the easiest, versatile, and most desirable of the DNA delivery methods, has been used in vitro for decades. The general principle is based on complex formation between positively charged chemicals (i.e., usually polymers) and negatively charged DNA molecules. These techniques can be broadly classified by the chemical involved in the complex formation, including calcium phosphate, artificial lipids, 2(diethylamino)ether (DEAE)-dextran, protein, dendrimers, and others. Information relevant to the delivery of nucleic acids, clinically and otherwise, using chemical means, can be found in, for example, U.S. Pat. Nos. 6,113,946; 6,169,078; 6,267,987; 6,344,446; 6,399,663; 6,716,882; 7,081,495; 7,153,905; and 7,309,757. While chemical transfection is conceptually appealing, each one of the preceding specifications suffers from one or more of the following disadvantages: (1) lack of cell-specific targeting, (2) poorly understood structure of delivery complexes, and (3) especially high toxicity which is seriously problematic, especially in clinical settings.

Because of their highly evolved and specialized components, viral vector systems are by far the most effective means of DNA delivery, achieving high efficiencies (i.e., usually >90%) for both delivery and expression. In fact, the majority of clinical gene therapy protocols either have or are currently using virus-based vectors for DNA delivery. Unfortunately, nearly all of the failures of these clinical applications can be attributed to the limitations of viral-mediated delivery. Information relevant to attempts to address nucleic acid delivery using viral vectors can be found in, for example, U.S. Pat. Nos. 5,728,379; 6,319,703; 6,335,011; 6,610,290; 6,746,860; 6,878,541; 7,241,617; and 7,314,614. Although viral vectors are highly efficient at delivering DNA into cells, each one of the preceding specifications suffers from one or more of the following disadvantages: (1) high toxicity, (2) restricted targeting of specific cell types, (3) limited DNA-carrying capacity, (4) production and packaging problems, (5) recombination, (6) potential for insertional mutagenesis, and (7) high cost. Further, the toxicity and immunogenicity of viral systems also hamper their routine use in basic research laboratories.

### Protection of Therapeutics

An area of significant importance in the delivery of therapeutic macromolecules is the necessity of their protection from proteolytic, nucleolytic, and immune degradation, while traversing extracellular spaces. For some applications, a possible solution to these and other problems is targeting drugs using carriers such as liposomes, niosomes, nanosuspensions, microspheres, nanoparticles, and micelles, etc. Information relevant to attempts to deliver drugs using these conventional carriers is extensive and includes, for example, U.S. Pat. Nos. 6,372,720; 6,383,500; 6,461,641; 6,569,528; 6,616,944; 7,001,614; 7,195,780; 7,288,266; and 7,345,138. However, when drug-carrying vessels reach a diseased target site using one or more of these conventional carriers—a feat that with present drug delivery technology is infrequent (depending on the therapeutic macromolecule and delivery)—in order to have any biologic or therapeutic effect, the drugs must typically gain entry into the cytoplasm of target cells. The present invention provides many novel methods and strategies to accomplish precisely this critical objective.

### Crossing Biological Barriers

Even though a close proximity of a therapeutic to many target cells can be achieved in some circumstances by employing various transport strategies—including the aforementioned vesicles—the plasma membrane of target cells, composed primarily of a bimolecular lipid matrix (i.e., mostly cholesterol and phospholipids), provides a formidable obstacle for both large and charged molecules. Thus, getting a drug across the plasma membrane into the cytosol, especially if enclosed in one of the aforementioned conventional carriers, is considered one of the greatest rate-limiting steps to intracellular drug delivery, as the majority of cells are not phagocytic and fusion of carriers with target cells is a very rare phenomenon. Unfortunately, the traditional route of internalization of many carriers and therapeutic macromolecules is by endocytosis, with subsequent degradation of the delivered therapeutic nucleic acids by lysosomal nucleases, strongly limiting the efficacy of most approaches known in the art. From this perspective alone, the development of a new, broadly applicable methodology, which can deliver genetic constructs and many other therapeutic macromolecules and other compounds directly into the cytoplasm of target cells in vivo, is highly desirable, both for use in clinical and laboratory settings.

Many organisms have developed processes for introducing macromolecules into living cells, and researchers are exploiting these methods for intracellular drug delivery. Aside from the cell-specific, usually receptor-mediated or active-uptake mechanisms, the major mechanism relies on peptides that have evolved to interact with and insert into lipid bilayer membranes. Drug delivery strategies utilize these peptides to cross both the plasma membrane bounding the cell, as well as intracellular membranes (e.g., membranes enclosing endocytic and other vesicles). These peptides include bacterial colicins, human porins, protein transduction domains (PTDs), and the like, from diverse species. Most of these compounds share the motif of a positively charged alpha-helix, frequently with an amphipathic structure, which is capable of inserting into lipid membranes and delivering larger cargoes intracellularly. Information relevant to attempts in intracellular drug delivery using these strategies may be found, for example, in U.S. Pat. Nos. 6,632,671; 6,780,846; 6,872,406; 7,087,729; 7,115,380; and 7,268,214, However, there are significant problems with using this type of approach for the intracellular delivery of pharmaceuticals, such as, for example, the specificity of this type of targeting to particular sites and structures, which greatly limits the technique's clinical application, as well as limiting many derivative methodologies.

### Reduction of Side Effects

Lastly, a common reason for side effects associated with many therapeutics is the high dosages required for most effective treatments. Typically, even modern pharmaceuticals do not accumulate selectively in disease areas and target tissues. Rather, following administration, therapeutics are more or less evenly distributed throughout the body. In order to generate clinically significant concentrations of drug at their desired site of action, a high concentration of the drug is typically administered. Doing this has the potential to cause undesirable complications and can be prohibitively expensive, especially considering the cost of most modern therapeutics. Therefore, development of effective drug delivery systems, such as embodiments of the present invention, that can transport and deliver a drug precisely and safely to its intended site of action, remains greatly sought after by modern medicine and scientific researchers.

### SUMMARY

Embodiments of the present invention are directed to methods and apparatuses with broad application in gene and drug delivery, satisfying the need for the delivery of a wide variety of pharmaceuticals intracellularly, most preferably nucleic acid therapeutics. Preferred embodiments are delivery systems designed to achieve (1) specific drug and gene targeting and (2) noninvasive, high-precision, in vivo intracellular drug delivery to selected cells and tissues. This is accomplished first by using, for example, self-assembling nanocarriers comprised substantially of biodegradable triblock copolymers, which may or may not be actively targeted, and mixtures thereof to safely carry therapeutics to the target site of the patient. The active targeting of said nanocarriers may be achieved using, for example, ligands such as antibody fragments or nucleic acid aptamers, or, in yet another preferred embodiment, guided by a magnetic field.

Once the nanocarriers are in the treatment area, drug delivery will commence following nanocarrier disassociation by high-intensity ultrasound (HIFU). Contrast agents are also used in embodiments of the invention to amplify, assist in controlling, and minimize tissue damage from acoustic cavitation in vivo. Thus, noninvasive sonic energy being applied to the patient in the treatment area, directly and indirectly, results in both therapeutic release from carrier vesicles, and cell- and tissue-specific drug delivery. Importantly, this is a delivery method that avoids the endocytic pathway(s) and many other biological barriers to efficient intracellular drug delivery, theoretically maximizing therapeutic efficacy. Another important embodiment of the invention is the delivery of therapeutics to organelles inside target cells, such as, for example, mitochondria, as well as to specific organs or organ regions, such as the anterior and posterior portion of the eye. Additional preferred embodiments include enclosing said nanocarriers in an acoustically responsive drug-delivery polymer matrix (e.g., a hydrogel).

### BRIEF DESCRIPTION OF THE DRAWINGS

The teachings of the present specification may be better understood, as well as its numerous features, benefits, and advantages made apparent to those skilled in the art, by referencing the accompanying drawings:

FIG. 1. Illustrates an embodiment of the nanocarriers of this specification.

FIG. 2A. Illustrates a cross-section of a capillary wall of the patent following administration with drug-containing nanocarriers and ultrasound contrast agents.

FIG. 2B. Illustrates a magnified view of a small section of FIG. 2A, showing a drug-containing nanocarrier bound onto the membrane of a cell bordering a capillary of the patient.

FIG. 3A. Illustrates the cross-section of a capillary wall in FIG. 2A, following exposure of said area to continuous acoustic energy. Said exposure causes acoustic cavitation, nanocarrier rupture, therapeutic release, and cell and membrane permeation, allowing therapeutics to diffuse into the cytoplasm of target cells.

FIG. 3B. Illustrates a magnified view of a small section of FIG. 3A, following exposure of said area to continuous acoustic energy. Said exposure causes acoustic cavitation, nanocarrier rupture, therapeutic release, and cell and membrane permeation, allowing therapeutics to diffuse into the cytoplasm of target cells.

FIG. 4. Illustrates the Fluid Mosaic Model of membrane structure (Singer et al., 1972), an appreciation of which is important in understanding the importance of this specification.

FIG. 5. Illustrates some of the basic processes of the endocytic pathway, an appreciation of which is important in understanding the importance of this specification.

FIG. 6. Illustrates the basic components of an ultrasonic sound wave.

FIG. 7. Illustrates a flowchart showing a preferred methodology for practicing the present invention.

FIG. 8A-C. Illustrate a preparation method for a single preferred method for practicing the present invention.

FIG. 9. Illustrates the sterile filtration in the final preparation step before administration of the assembled nanocarriers to the patient, where said vesicles contain one or more therapeutics.

FIG. 10. Illustrates prospective data describing the encapsulation efficiency of nanocarriers comprised substantially of biodegradable triblock copolymers.

FIG. 11. Illustrates a prospective experimental system that is used for evaluating parameters that may impact ultrasonic drug delivery.

FIG. 12A-12C. Illustrate prospective data describing the impact of contrast agent concentration, ultrasonic pressure and exposure time on cell viability (i.e., white bars) and the percentage with calcein uptake (i.e., black bars) following nanocarrier rupture employing the experimental system of #FIG. 11.

FIG. 13A-13B. Illustrates prospective data describing the impact of ultrasonic pressure and exposure time, in the presence of contrast agent, on HeLa viability (i.e., white bars) and the percentage with calcein uptake (i.e., black bars) following nanocarrier rupture employing the experimental system of #FIG. 11.

FIG. 13C-13D. Illustrates prospective data describing the impact of ultrasonic pressure and exposure time, in the presence of contrast agent, on AoSMC cell viability (i.e., white bars) and the percentage with calcein uptake (i.e., black bars) following nanocarrier rupture employing the experimental system of #FIG. 11.

### DESCRIPTION OF THE PREFERRED EMBODIMENTS

Unless defined otherwise, all technical and scientific terms used herein generally have the same meaning as commonly understood by one of ordinary skill in the art to which embodiments of the present invention belong. Generally, the nomenclature used herein, unless specifically defined below, and the clinical and laboratory procedures in cell culture, molecular genetics, organic chemistry, polymer chemistry, nucleic acid chemistry, and diagnostic ultrasound are those well known and commonly employed in the art. In addition, the techniques and procedures are generally performed according to conventional methods in the art. Throughout this specification, various general references describing said techniques and procedures are provided primarily for enablement purposes.

### DEFINITIONS

“Acoustic” generally refers to processes or procedures having to do with the generation, transmission, focusing, sensitivity, and disposition of sound wave energy.

“Acoustic energy” refers to any form of pressure wave, whether audible or inaudible. The frequency of the acoustic energy can be a single frequency or a combination of frequencies. The range of useful frequencies preferably is between approximately 1 Hz and 100 MHz, and more preferably is between approximately 15 kHz and 2 MHz. The waveform of the acoustic energy can be of any shape including a sinewave or a combination of sinewaves. The pressure of the acoustic energy can be up to a few hundred atmospheres, and preferably is applied at a peak positive pressure of up to 100 atmospheres. The optimal pressure is a function of acoustic frequency and other parameters detailed herein. The acoustic energy can be applied continuously, intermittently (e.g., pulsed), or a combination thereof.

“Acoustic sensitivity,” “acoustic responsiveness” “ultrasonically sensitive,” or “ultrasonic sensitivity” of a compound, polymer, copolymer, structure or other material, etc., is generally used herein to refer to materials described in detail under the definition of “ultrasonically sensitive materials.”

As used herein, “administering” means oral administration, administration as a suppository, topical contact, intravenous, intraperitoneal, intramuscular, intralesional, intranasal, or subcutaneous administration, or the implantation of a slow-release device (e.g., a mini-osmotic pump) to the patient. Administration is by any route including parenteral and transmucosal (e.g., oral, nasal, vaginal, rectal, or transdermal). Parenteral administration includes, for example, intravenous, intramuscular, intra-arteriole, intradermal, subcutaneous, intraperitoneal, intraventricular, and intracranial. Moreover, where injection is to treat a tumor (e.g., induce apoptosis), administration may be directly to the tumor and/or into tissues surrounding the tumor.

An “amphiphile” or “amphipathic” chemical species refers to a chemical compound possessing both a hydrophilic and hydrophobic nature. Molecules of amphiphilic compounds have hydrophobic (i.e., usually of a hydrocarbon nature) and hydrophilic structural regions (i.e., represented by either ionic or uncharged polar functional groups). Phospholipids, a double-chain class of amphiphilic molecules, are the main components of biological membranes. The amphiphilic nature of these molecules defines the way in which they form membranes. They arrange themselves into bilayers, by positioning their polar groups toward the surrounding aqueous medium, and their hydrophobic chains toward the inside of the bilayer, defining a non-polar region between two polar ones. Although phospholipids are the principal constituents of biological membranes, there are other amphiphilic molecules (e.g., cholesterol and glycolipids), which are also included in animal cell membranes, giving them different physical and biological properties. Many other amphiphilic compounds strongly interact with biological membranes by insertion of a hydrophobic part into the lipid membrane, while exposing the hydrophilic portion to an aqueous medium, altering the membrane's physical behavior and sometimes disrupting the membrane. For example, surfactants are an example group of amphiphilic compounds where their polar region can be either ionic or non-ionic. Some typical members of this group include sodium dodecyl sulphate (i.e., anionic), benzalkonium chloride (i.e., cationic), cocamidopropyl betaine (i.e., zwitterionic), and octanol (i.e., long-chain alcohol, non-ionic). In addition, many biological compounds are amphiphilic by nature (e.g., phospholipids, cholesterol, glycolipids, fatty acids, bile acids, saponins). Many components of the preferred embodiments of the present invention are either themselves amphiphilic or contain amphiphilic components.

“Aptamer” generally refers to a single-stranded, partially single-stranded, partially double-stranded, or double-stranded nucleotide sequence, advantageously a replicatable nucleotide sequence, capable of specifically recognizing a selected nonoligonucleotide molecule or group of molecules by a mechanism other than Watson-Crick base pairing or triplex formation. Aptamers referred to herein include, without limitation, defined sequence segments and sequences comprising nucleotides, ribonucleotides, deoxyribonucleotides, nucleotide analogs, modified nucleotides, and nucleotides comprising backbone modifications, branchpoints and nonnucleotide residues, groups, or bridges. Aptamers for use with embodiments of the present invention also include partially and fully single-stranded and double-stranded nucleotide molecules and sequences, synthetic RNA, DNA, chimeric nucleotides, hybrids, duplexes, heteroduplexes, and any ribonucleotide, deoxyribonucleotide, or chimeric counterpart thereof, and/or the corresponding complementary sequence, promoter or primer-annealing sequence needed to amplify, transcribe, or replicate all or part of the aptamer molecule or sequence. Unlike many prior art aptamers that specifically bind to soluble, insoluble, or immobilized selected molecules (e.g., ligands, receptors, effector molecules, etc.), in this specification, the term “aptamer” includes nucleotides capable of shape-specific recognition of surfaces by a mechanism distinctly different from specific binding. An aptamer may be a molecule unto itself or a sequence segment comprising a nucleotide molecule or group of molecules (e.g., a defined sequence segment or aptameric sequence comprising a synthetic heteropolymer or a multivalent heteropolymeric hybrid structure).

A “bilayer membrane” [or simply “bilayer(s)”] refers to a self-assembled membrane of amphiphiles in an aqueous solution.

“Bioactive” refers to the ability of a therapeutic or other agent to interact with the patient, living tissue, cell, or other system. “Bioactive agent” refers to a substance which may be used in connection with an application that is therapeutic or diagnostic such as, for example, in methods for diagnosing the presence or absence of a disease in a patient and/or methods for the treatment of a disease in a patient. “Bioactive agent” also refers to substances which are capable of exerting a biological effect in vitro and/or in vivo. The bioactive agents may be neutral, positively, or negatively charged. Exemplary bioactive agents include, for example, (1) prodrugs, (2) targeting ligands, (3) diagnostic agents, (4) pharmaceutical agents, (5) drugs, (6) synthetic organic molecules, (7) proteins, (8) peptides, (9) vitamins, (10) steroids, (11) steroid analogs, and (12) genetic material (e.g., nucleosides, nucleotides, and polynucleotides).

“Biocompatible” generally refers to materials which are not injurious to biological functions and which will not result in any degree of unacceptable toxicity including allergenic responses and disease states in the patient. A biocompatible substance, when implanted in or juxtaposed against a living body or placed in contact with fluid or material actively leaving and reentering said body, does not cause an adverse pathophysiological event that would raise significant concerns about the health of said patient.

“Biodegradable” or a “biodegradable substance” refers generally to a substance that when in a living body and/or in contact with the patient will, over a period of time, disintegrate and/or decompose in a manner, for example, that alleviates the necessity for a procedure to remove said substance from said patient. Biodegradation may result from active processes such as enzymatic means or from spontaneous (e.g., non-enzymatic) processes such as the chemical hydrolysis of, for example, ester bonds of polylactides that occur at bodily temperature in an aqueous solution.

“Biodendrimer” or “biodendritic macromolecules” generally refers to a class of dendritic macromolecules, composed entirely, or almost entirely of building blocks known to be biocompatible or biodegradable to natural metabolites in vivo. These biocompatible or natural metabolite monomers include, but are not limited to, glycerol, lactic acid, glycolic acid, succinic acid, ribose, adipic acid, malic acid, glucose, and citric acid.

“Biodistribution” refers to the pattern and process of a chemical substance's distribution throughout the tissues, cells, and other bodily structures or fluids of the patient.

“Block copolymer” refers to a polymer with at least two tandem, interconnected regions of differing chemistry (i.e., “blocks”). Each region is comprised of a repeating sequence of monomers. Thus, a diblock copolymer is comprised of two such connected regions (i.e., A-B); a triblock copolymer is comprised of three such connected regions (i.e., A-B-C). For example, PS-β-PMMA is short for polystyrene-β-poly(methyl methacrylate); it is made by first polymerizing styrene and then subsequently polymerizing MMA. This polymer is a diblock copolymer because it contains two different chemical blocks. Triblocks, tetrablocks, pentablocks, etc., can also be synthesized. Diblock copolymers may be synthesized, for example, using living polymerization techniques such as atom transfer free radical polymerization (ATRP), reversible addition fragmentation chain transfer (RAFT), living cationic or living anionic polymerizations, etc. Block copolymers are especially important in many embodiments of the present invention because they can microphase separate to form periodic nanostructures. If there is a hydrophobic first block and a hydrophilic second block, the block copolymers undergo microphase separation, where the hydrophobic and hydrophilic blocks form nanometer-sized structures. The interaction parameter, also called “chi” (χ), gives an indication of how different, chemically, the two blocks are and whether or not they will microphase separately. If the product of χ and the molecular weight are large (i.e., >10.5), the blocks will likely microphase separately. If the product of χ and the molecular weight are too small (i.e., <10.5), the different blocks are likely to mix. “Branched polymer” generally refers to polymers with side chains or branches of significant length, which are bonded to the main chain at branch points, also known as junctional points. Branch polymers are characterized in terms of the number and size of the branches. For the purposes of this specification, dendrimers, dendrons, and dendrigrafts are separate and distinct branched chain polymers that possess a full or partial dendritic or cascade architecture.

A “capsule” refers to the encapsulating membrane plus the space enclosed within the membrane. A “carrier” refers to a pharmaceutically acceptable vehicle which is a nonpolar, hydrophobic solvent, and which may serve as a reconstituting medium. The carrier may be aqueous-based or organic-based. Carriers include lipids, proteins, polysaccharides, sugars, polymers, copolymers, acrylates, and the like.

“Cavitation” or “acoustic cavitation” refers to the oscillation of bubbles in an acoustic field, as well as the sequential formation and collapse of vapor bubbles, voids, and in many embodiments of the present invention, microbubbles, in a liquid, including liquids within or composing the patient, subjected to acoustic energy. Cavitation is usually divided into two classes of behavior (1) inertial (i.e., transient or collapse) cavitation and (2) gas body activation (i.e., non-inertial) cavitation. “Inertial cavitation,” “transient cavitation,” or “collapse cavitation” refers to the process where a void or a bubble in a liquid rapidly collapses, producing a shock wave. Such cavitation often occurs in pumps, propellers, impellers, and in the vascular tissues of plants. Non-inertial or “gas body activation” (i.e., formerly “stable cavitation”) refers to the process where a bubble in a fluid is forced to oscillate in size or shape due to some form of energy input such as, for example, an acoustic field. This phenomenon is analogous to thermal boiling but without the associated rise in temperature of a liquid mass, although localized temperatures on the molecular level can be extremely high. A “cavitation field” refers to that volume, within a processing container, flow system, or biological system—including the patient—in which active cavitation is generated. Other forces they may be acting up drug delivery vesicles exposed to HIFU include radiation forces. For the purposes of this specification, said radiation forces are considered distinctly different than the influences on drug delivery vesicles caused by acoustic cavitation.

A “cell” refers to any one of the minute protoplasmic masses which makes up organized mammalian or other tissues, including those of the patient, comprising a mass of protoplasm surrounded by a membrane, including nucleated and unnucleated cells and organelles. The “cell membrane” or “plasma membrane” refers to a complex, contiguous, self-assembled, complex fluid structure comprised of amphiphilic lipids in a bilayer, with associated proteins, and which defines the boundary of every cell. The structure is also referred to as a “biomembrane” Phospholipids comprise lipid substances which occur in cellular membranes and contain esters of phosphoric acid such as sphingomyelins, and include phosphatides, phospholipins, and phospholipoids.

“Clinical” generally refers to a clinic, or conducted in or as if in a clinic, where “clinic” refers to a medical establishment run by a group of medical specialists, where medical or healthcare problems and concerns are diagnosed, and solutions possibly devised, as well as where remedial work may be performed.

“Complex fluids” are fluids that are made from molecules that interact and self-associate, conferring novel technological, optical, or mechanical properties on the fluid itself. Complex fluids are found throughout biological and chemical systems, and include materials such as biological membranes or biomembranes, polymer melts and blend, and liquid crystals. The self-association and ordering of the molecules within the fluid depend on the interaction between component parts of the molecules, relative to their interaction with the solvent, if present.

As used herein, the transitional term “comprising,” which is synonymous with “including,” “containing,” or “characterized by,” is inclusive or open-ended and does not exclude additional, unrecited elements, method steps, or the like, in, for example, a patent claim. The transitional phrase “consisting of” excludes any element, step, or ingredient not specified in, for example, a patent claim. Further, a patent claim, for example, which depends on a patent claim which “consists of” the recited elements or steps cannot add an element or a step. When the phrase “consists of” appears in a clause, for example, of the body of a patent claim, rather than immediately following the preamble, it limits only the element set forth in that clause; other elements are not excluded from the claim as a whole. The transitional phrase “consisting essentially of” limits, for example, the scope of a patent claim to the specified materials or steps and those that do not materially affect the basic and novel characteristic(s) of an invention. A “consisting essentially of” patent claim occupies a middle ground between closed claims that are written in a “consisting of” format and fully open, for example, patent claims that are drafted in a “comprising” format.

“Contrast agent” generally refers to a vesicle or compound that is injected into the body of the patient to make certain tissues more visible during diagnostic imaging (e.g., ultrasound, angiography, computer topography [CT], myelogram, magnetic resonance imaging [MRI], and the like).

The term “microbubble” may be used interchangeably with “contrast agent.” As exemplified in many of the preferred embodiments of the present invention, gaseous ultrasound and/or other contrast agents may be used as, or in conjunction with, therapeutic procedures or processes.

“Controlled delivery” or “controlled release” refers to delivery of a substance by a device in a manner that affords control by said device over the rate and duration of the exit of said substance from said device. For example, delivery from controlled release devices can be modulated by diffusion out of a device, dissociation of chemical bonds, and the like.

The term “copolypeptide” or “block copolypeptide” refers to polypeptides containing at least two covalently linked domains (“blocks”), one block having amino acid residues that differ in composition from the composition of amino acid residues of another block. The number, length, order, and composition of these blocks can vary to include all possible amino acids in any number of repeats. Preferably the total number of overall monomer units (i.e., residues) in the block copolypeptide is greater than 100, and the distribution of chain-lengths in the block copolymer is approximately 1.01<M_{w}/Mₙ<1.25, where M_{w}/Mₙ=weight average molecular weight divided by the number average molecular weight.

“Cooperative non-covalent bonding” refers to interactions between two or more molecules or substances that result from two or more non-covalent chemical bonds; among them, hydrogen bonds, ionic bonds, hydrophobic interactions, and the like.

“Covalent bond” or “covalent association” refers to an intermolecular association or bond which involves the sharing of electrons in the bonding orbitals of two atoms.

“Cross-link,” “cross-linked,” or “cross-linking” generally refers to the linking of two or more stabilizing materials, including lipid, protein, polymer, carbohydrate, surfactant stabilizing materials, and/or bioactive agents, by one or more bridges. The bridges may be composed of one or more elements, groups, or compounds, and generally serve to join an atom from first a stabilizing material/molecule to an atom of a second stabilizing material/molecule. The cross-link bridges may involve covalent and/or non-covalent associations. Any of a variety of elements, groups, and/or compounds may form said bridges in the cross-links, and the stabilizing materials may be cross-linked naturally or through synthetic means. For example, cross-linking may occur in nature in material formulated from peptide chains which are joined by disulfide bonds of cysteine residues, as in keratins, insulins, and other proteins. Alternatively, cross-linking may be effected by suitable chemical modification such as, for example, by combining a compound such as a stabilizing material, and a chemical substance that may serve as a cross-linking agent, which may cause to react by, for example, exposure to heat, high-energy radiation, ultrasonic radiation, and the like. Examples include cross-linking by sulfur to form disulfide linkages, cross-linking using organic peroxides, cross-linking of unsaturated materials by means of high-energy radiation, cross-linking with dimethylol carbamate, and the like. Photopolymerization represents a preferred method of cross-linking the polymers and/or other structures comprising the nanocarriers of embodiments of the present invention. If desired, the stabilizing compounds and/or bioactive agents may be substantially cross-linked. The term “substantially” means that greater than approximately 50% of the stabilizing compounds contain cross-linking bridges. If desired, greater than approximately 60%, 70%, 80%, 90%, 95%, or even 100% of the stabilizing compounds contain such cross-linking bridges. Alternatively, the stabilizing materials may be non-cross-linked (i.e., such that greater than approximately 50% of the stabilizing compounds are devoid of cross-linking bridges) and, if desired, greater than approximately 60%, 70%, 80%, 90%, 95%, or even 100% of the stabilizing compounds are devoid of cross-linking bridges.

“Cytotoxicity” refers to the quality of being toxic to living cells or tissues of for example, those composing or belonging to the patient. Examples of toxic agents are chemical substances, as well as physical processes or procedures such as, for example, thermal treatment, or from exposure to natural agents such as, for example, immune cells.

A “dendrigraft” generally refers to “hyper comb-branched,” “hyperbranched,” and “non-symmetrical hyperbranched” polymers. These may comprise non cross-linked, poly branched polymers prepared by, for example, (1) forming a first set of linear polymer branches by initiating the polymerization of a first set of monomers, which are either protected against or non-reactive to branching and grafting, during polymerization, each of the branches having a reactive end unit upon completion of polymerization, the reactive end units being incapable of reacting with each other; (2) grafting the branches to a core molecule or core polymer having a plurality of reactive sites capable of reacting with the reactive end groups on the branches; (3) either deprotecting or activating a plurality of monomeric units on each of the branches to create reactive sites; (4) separately forming a second set of linear polymer branches by repeating step (1) with a second set of monomers; and (5) attaching the second set of branches to the first set of branches by reacting the reactive end groups of the second set of branches with the reactive sites on the first set of branches, and then repeating steps (3), (4), and (5) above to add one or more subsequent sets of branches.

A “dendrimer” refers to a dendritic polymer in which the atoms are arranged in many branches and subbranches along a central backbone of carbon atoms, with perfect dendrimers having an f_{br}=1.0, where dendrimers follow a dendritic or cascade architecture. Dendrimers are also called cascade molecules with a form like the branches of a tree. The name comes from the Greek ‘δενδρoν’/dendron, meaning “tree” The structures were first synthesized in 1981 (U.S. Pat. Nos. 4,410,688 and 4,507,466). In their synthesis, monomers lead to a monodispersed tree like polymer or generational structure. There are two defined methods of dendrimer synthesis (1) divergent and (2) convergent synthesis. The former assembles the molecule from the core to the periphery, and the latter from the outside, terminating at the core. The properties of dendrimers are dominated by the functional groups on their molecular surface. For example, a dendrimer can be water-soluble when its end-group is hydrophilic, like a carboxyl group. The inside of a dendrimer has a unique chemical microenvironment because of its high density.

“Dendritic polymer” and “dendritic” generally refer to polymers characterized by a relatively high degree of branching, which is defined as the number average fraction of branched groups per molecule (i.e., the ratio of terminal groups plus branch groups to the total number of terminal groups, branched groups, and linear groups). For ideal dendrons and dendrimers, the degree of branching is 1; for linear polymers, the degree of branching is 0. “Hyperbranched polymers” have a degree of branching that is intermediate to that of linear polymers and ideal dendrimers—preferably of at least 0.5 or higher. The degree of branching is expressed in the following equation

$${f}_{b} = \frac{{N}_{t} + {N}_{b}}{{N}_{t} + {N}_{b} + {N}_{1}} \qquad Equation 1$$

wherein Nₓ is the number of type x units in the structure. Both terminal (type t) and branched (type b) units contribute to the fully branched structure, while linear (type 1) units reduce the branching factor. Therefore, 0≦f_{br}≦1; where f_{br}=O represents the case of a linear polymer, and f_{br}=1 represents the case of a fully branched macromolecule.

A “dendrisome” generally refers to a vesicle formed from dendritic polymers, a structure that is capable of transporting hydrophilic as well as hydrophobic therapeutics. Dendrisomes are reminiscent of cationic liposomes, except that no cationic lipid is added to impart a positive charge. Dendrisomes may be, for example, supramolecular complexes, or may be stabilized or otherwise crosslinked. A “dendron” generally refers to polymeric structures that can be broadly classified as “partial” dendrimers and represent a diverse number of compounds with widely varying characteristics. This variety of structures has led to systems which have the ability to self-associate or to form with agents such as surfactants and lipids. The self-assembly of dendrons can involve hydrogen bonding, hydrophobic, or electrostatic interactions. Self-assembly can also be directed by a template which interacts with functional group(s) on the dendron. Such interactions can be mediated by ligand-metal interactions, hydrogen bonding, or electrostatic interactions.

A “diagnostic agent” refers to any agent which may be used in connection with methods for imaging an internal region of the patient and/or diagnosing the presence or absence of a disease in the patient. Exemplary diagnostic agents include, for example, contrast agents for use in connection with ultrasound imaging, magnetic resonance imaging (MRI), or computed tomography (CT) imaging of the patient. Diagnostic agents may also include any other agents useful in facilitating diagnosis of a disease or other condition in a patient, whether or not an imaging methodology is employed. “Dipole-dipole interaction” refers to the attraction of the uncharged, partial positive end of a first polar molecule, commonly designated as δ⁺ to the uncharged, partial negative end of a second polar molecule commonly designated as δ⁻. Dipole-dipole interactions are exemplified by the attraction between the electropositive head group, for example, the choline head group, of phosphatidylcholine, and an electronegative atom, for example, a heteroatom such as oxygen, nitrogen, or sulfur, which is present in a stabilizing material such as a polysaccharide. “Dipole-dipole interaction” also refers to intermolecular hydrogen bonding in which a hydrogen atom serves as a bridge between electronegative atoms on separate molecules, and in which a hydrogen atom is held to a first molecule by a covalent bond and to a second molecule by electrostatic forces.

“Droplet” refers to a spherical or spheroidal entity which may be substantially liquid or which may comprise liquid and solid; solid and gas; liquid and gas; or liquid, solid, and gas. Solid materials within a droplet may be, for example, particles, polymers, lipids, proteins, or surfactants. “Dry” and variations thereof, refer to a physical state that is dehydrated or anhydrous, (i.e., substantially lacking liquid). Drying includes, for example, spray drying, lyophilization, and vacuum drying.

The abbreviation “e.g.” refers to the phrase “for example.”

“Emulsion” refers to a mixture of two or more generally immiscible liquids, and is generally in the form of a colloid (i.e., suspension). The mixture may be of polymers, for example, which may be homogeneously or heterogeneously dispersed throughout the emulsion. Alternatively, the polymers may be aggregated in the form of, for example, clusters or layers, including monolayers or bilayers, as embodied by many of the nanocarriers of this specification. Immiscible liquids can sometimes remain mixed by the addition of an emulsifier. An “emulsifier”, also known as a surfactant or emulgent, refers to a substance which stabilizes an emulsion. A wide variety of emulsifiers are used in formulating therapeutics for the patient such as, for example, propofol, polysorbates, and sorbitan esters, to prepare vehicles, creams, and lotions. Generally, the Bancroft rule applies: Emulsifiers and emulsifying particles tend to promote dispersion of the phase in which they are not very soluble; for example, proteins dissolve better in water than in oil, and so tend to form oil-in-water emulsions (i.e., they promote the dispersion of oil droplets throughout a continuous phase of water). An “encapsulating membrane” refers to a vesicle, in all respects, except for the necessity of an aqueous solution.

The abbreviation “etc.” refers to “et cetera,” which is Latin for “and the others,” and is generally used herein to represent the logical continuation of some sort of series. The abbreviation “et al.” is used in place of etc., in lists of persons such as authors and inventors of for example, peer-reviewed research publications and patents, respectively.

“Extracellular” or “extracellular space” generally refers to the area or region outside of an animal cell. This space is usually taken to be outside the plasma membranes and is occupied by fluid. The term is used in contrast to intracellular. The cell membrane is the barrier between the extra- and intracellular regions, and the chemical composition of extra- and intracellular milieu can be radically different. The composition of the extracellular space includes metabolites, ions, proteins, and many other substances that might affect cellular function. For example, hormones act by traveling in the extracellular space toward cell receptors. Other proteins that are active outside the cell are, for example, digestive enzymes. The term “extracellular” is often used in reference to the extracellular fluid (ECF) which composes approximately 15 liters of the average human body.

“For example” refers to an illustrative instance. At no time should the phrase “for example” convey any type of limitation or exclusive enumeration.

“Genetic material” refers generally to nucleotides and polynucleotides, including deoxyribonucleic acid (DNA) and ribonucleic acid (RNA). The genetic material may be made by synthetic chemical methodology, known to one of ordinary skill in the art; or by the use of recombinant technology, or by a combination thereof. The DNA and RNA may optionally comprise unnatural nucleotides and may be single- or double-stranded. “Genetic material” also refers to sense and anti-sense DNA and RNA; that is, a nucleotide sequence which is complementary to a specific sequence of nucleotides in DNA and/or RNA, including RNA interference (RNAi), small interfering RNA (siRNA), aptamers, and the like.

“Graft copolymer” or “graft polymer” generally refers to a polymer having polymer chains, of one kind chemically bonded onto the sides of polymer chains with a different chemical composition. As with block copolymers, the quasi-composite product has properties of both “components.” Also called comb-type polymers, there are two general methods that have been applied to synthesize graft polymers, according to the properties of backbone and branching. One method refers to the direct copolymerization of two, or more than two, monomers, one of which must already have branching. The other method uses the polymers as a backbone in the presence of polyfunctional active sites which are used to couple with new branches or to initiate the propagation of branching. The use of graft polymers in delivery vesicles for acoustically mediated drug delivery represents a preferred embodiment of the present invention.

“HIFU” refers to an acronym for “high-intensity focused ultrasound” a minimally invasive medical technique used for a variety of procedures, including tumor ablation and destruction. HIFU technology is noninvasive and is used in both inpatient and outpatient facilities. An extracorporeal applicator generates a powerful, converging beam of ultrasound rays, focusing on a very precise point on the exterior or, preferably, inside the body of the patient. When the volume is larger, a sweep is performed with successive, juxtaposed exposures. Depending on conventional instrumentation parameters and other variables, concentrated acoustic energy causes a very rapid rise in temperature at this exact focal point. Outside of this point, the temperature remains normal. Importantly, in the preferred embodiments of the present invention, HIFU is used in primarily mediating intracellular drug delivery in vivo. This is accomplished, generally, by utilizing HIFU for initiating, maintaining, and controlling acoustic cavitation. Importantly, one of the goals in this type of application is little or no overall increase in temperature at the target region associated with said ultrasonic exposure.

“Hybrid” refers to a composite of mixed content or origin.

“Hydrogen bond” refers to an attractive force, or bridge, which may occur between a hydrogen atom which is bonded covalently to an electronegative atom; (e.g., oxygen, sulfur, or nitrogen) and another electronegative atom. The hydrogen bond may occur between a hydrogen atom in a first molecule and an electronegative atom in a second molecule (i.e., intermolecular hydrogen bonding). Also, the hydrogen bond may occur between a hydrogen atom and an electronegative atom, which are both contained in a single molecule (i.e., intramolecular hydrogen bonding).

“Hydrophilic” or “hydrophilic interaction” generally refers to molecules or portions of molecules which may substantially bind with, absorb, and/or dissolve in water. This may result in swelling and/or the formation of reversible gels. “Hydrophobic” or “hydrophobic interaction” generally refers to molecules or portions of molecules which do not substantially bind with, absorb, and/or dissolve in water.

The abbreviation “i.e.” refers to the phrases “that is (to say),” “in other words,” or sometimes, or “in this case,” depending on the context.

“Including” or “includes” refers to enlargement, have as a part, be made up of not of exclusive enumeration, and without limitation of any kind.

“Ionic interaction” or “electrostatic interaction” refers to intermolecular interaction among two or more molecules, each of which is positively or negatively charged. Thus, for example, “ionic interaction” or “electrostatic interaction” refers to the attraction between a first, positively charged molecule and a second, negatively charged molecule. Ionic or electrostatic interactions include, for example, the attraction between a negatively charged stabilizing material (e.g., genetic material and a positively charged polymer). “In combination with” refers to the incorporation of, for example, bioactive agents, therapeutics, and/or targeting ligands, in a composition of embodiments of the present invention, including emulsions, suspensions, and vesicles. The therapeutic, bioactive agent, and/or targeting ligand can be combined with the therapeutic delivery system, and/or stabilizing composition(s), including vesicles, in a variety of ways. For example, the therapeutic, bioactive agent and/or targeting ligand may be associated covalently and/or non-covalently with the delivery system or stabilizing material(s). Further, the therapeutic, bioactive agent and/or targeting ligand may be entrapped within the internal void(s) of the delivery system or vesicle. The therapeutic, bioactive agent and/or targeting ligand may also be integrated within the layer(s) or wall(s) of the delivery system or vesicle, for example, by being interspersed among stabilizing material(s) which form, or are contained within, the vesicle layer(s) or wall(s). In addition, it is contemplated that the bioactive agent and/or targeting ligand may be located on the surface of a delivery system or vesicle or non-vesicular stabilizing material. The therapeutic, bioactive agent and/or targeting ligand may be concurrently entrapped within an internal void of the delivery system or vesicle and/or integrated within the layer(s) or wall(s) of the delivery vesicles and/or located on the surface of a delivery vesicle or non-vesicular stabilizing material. In any case, the therapeutic, bioactive agent and/or targeting ligand may interact chemically with the walls of the delivery vesicles, including, for example, the inner and/or outer surfaces of the delivery vesicle, and may remain substantially adhered thereto. Such interaction may take the form of for example, non-covalent association or bonding, ionic interactions, electrostatic interactions, dipole-dipole interactions, hydrogen bonding, van der Waal's forces, covalent association or bonding, cross-linking, or any other interaction, as will be readily apparent to one skilled in the art, in view of the present disclosure. In certain embodiments, the interaction may result in the stabilization of the vesicle. The bioactive agent may also interact with the inner or outer surface of the delivery system or vesicle or the non-vesicular stabilizing material in a limited manner. Such limited interaction would permit migration of the bioactive agent, for example, from the surface of a first vesicle to the surface of a second vesicle, or from the surface of a first non-vesicular stabilizing material to a second non-vesicular stabilizing material. Alternatively, such limited interaction may permit migration of the bioactive agent, for example, from within the walls of the delivery system, vesicle and/or non-vesicular stabilizing material to the surface of the delivery system, vesicle and/or non-vesicular stabilizing material, and vice versa, or from inside a vesicle or non-vesicular stabilizing material to within the walls of a vesicle or non-vesicular stabilizing material, and vice versa.

“Insonate,” and variations thereof, refers to exposing, for example, regions of the patient to ultrasonic waves. The term “interpolymer” refers to a polymer comprising at least two types of monomers and, therefore, encompasses copolymers, terpolymers, and the like.

“Intracellular” or “intracellularly” refers to the area enclosed by the plasma membrane of a cell including the protoplasm, cytoplasm, and/or nucleoplasm.

“Intracellular delivery” refers to the delivery of a bioactive agent such as, for example, a therapeutic, into the area enclosed by the plasma membrane of a cell.

“Laboratory” or “lab” generally refers to a place where scientific research and experiments are conducted.

“Lipid” refers to a naturally occurring synthetic or semi-synthetic (i.e., modified natural) compound which is generally amphipathic. The lipids typically comprise a hydrophilic component and a hydrophobic component. Exemplary lipids include, for example, fatty acids, neutral fats, phosphatides, oils, glycolipids, surface-active agents (i.e., surfactants), aliphatic alcohols, waxes, terpenes, and steroids. The phrase semi-synthetic (i.e., or modified natural) denotes a natural compound that has been chemically modified in some fashion. In this application, lipids are differentiated from other amphipathic compounds by having two hydrophobic “chains.” A “lipid bilayer” refers to a eucaryotic (i.e., animal) cell plasma membrane which comprises a double layer of phospholipid/diacyl chains, wherein the hydrophobic fatty acid tails of the phospholipids face each other and the hydrophilic polar heads of each layer face outward toward the aqueous solution. Numerous receptors, steroids, transporters, and the like are embedded within the bilayer of a typical cell. Throughout this specification, the terms “cell membrane,” “plasma membrane,” “lipid membrane,” and “biomembrane” may be used interchangeably to refer to the same lipid bilayer surrounding an animal cell.

A “liposome” refers to a generally spherical or spheroidal cluster or aggregate of amphipathic compounds, composed mainly of phospholipids, typically in the form of one or more concentric layers (e.g., bilayers). They may also be referred to herein as lipid vesicles. The liposomes may be formulated, for example, from ionic lipids and/or non-ionic lipids.

A “liposphere” refers to an entity comprising a liquid or solid oil, surrounded by one or more walls or membranes, with a gaseous central core.

“Lyphilized,” or freeze drying, refers to the preparation of a polymer or other composition in dry form by rapid freezing and dehydration in the frozen state, sometimes referred to as sublimation. Lyophilization takes place at a temperature resulting in the crystallization of the composition to form a matrix. This process may take place under a vacuum at a pressure sufficient to maintain the frozen product with the ambient temperature of the containing vessel at approximately room temperature; preferably less than 500 mTorr; more preferably less than approximately 200 mTorr; and even more preferably, less than 1 mTorr.

“Megahertz (MHz)” refers to a unit of frequency equivalent to one million “cycles per second” (cps). One Megahertz (1 MHz) equals 1,000,000 cps.

A “membrane” refers to a spatially distinct collection of molecules that defines a 2-dimensional surface in 3-dimensional space, and thus separates one space from another in at least a local sense. Such a membrane must also be semi-permeable to solutes. Said membrane must also be submicroscopic (i.e., less than optical wavelengths of around 500 nm) in thickness, resulting from a process of self-assembly. Said membrane can have fluid or solid properties, depending on temperature and on the chemistry of the amphiphiles from which it is formed. At some temperatures, the membrane can be fluid (i.e., having a measurable viscosity), or it can be solid-like, with an elasticity and bending rigidity. The membrane can store energy through its mechanical deformation, or it can store electrical energy by maintaining a transmembrane potential. Under certain conditions, membranes can adhere to each other and coalesce (i.e., fuse). Soluble amphiphiles can bind to and intercalate within a membrane.

A “micelle” refers to colloidal entities formulated from primarily single-chain amphiphiles; in several preferred embodiments, a micelle is designed for a specified level of acoustic sensitivity. In certain preferred embodiments, the micelles comprise a monolayer, bilayer, or other structure.

A “microbubble” refers to a gaseous ultrasound contrast agent bounded by one or more membranes.

A “mixture” refers to the product of blending or mixing of chemical substances like elements, compounds, and other structures, including the nanocarriers of embodiments of the present disclosure, comprised, for example, of different polymers containing the same or different therapeutics, usually without chemical bonding or other chemical change, so that each ingredient and substance retains its own chemical properties and makeup. While there are frequently no chemical changes in a mixture, physical properties of a mixture may differ from those of its components. Mixtures can usually be separated by mechanical means. The term “mixture” includes solutions, homogeneous mixtures, heterogeneous mixtures, emulsions, colloidal dispersions, suspensions, dispersions, and the like.

“Mole fraction,” “mole percent,” and “mole %” refer to a chemical fraction defined as apart over a whole. Thus, a mole fraction involves knowing the moles of a solute, or component of interest (i.e., a particular copolymer species), over the total moles of all component(s) in a system (i.e., total nanocarrier components in a mixture). Multiplying the fraction calculated with the equation below by 100 yields the “mole percent.”

$${X}_{solute} = \frac{moles of solute}{total moles of all components} \qquad Equation 2$$

A “nanocarrier” refers to a vesicular (i.e., vesicle) embodiment of the present invention that may or may not be acoustically responsive and capable of being disrupted, temporarily, permanently, completely, or in part, with ultrasonic energy, having a diameter generally between 20 nm and 1,000 nm (1 μm).

“Non-covalent bond” or “non-covalent association” refers to intermolecular interaction, among two or more separate molecules, which does not involve a covalent bond. Intermolecular interaction is dependent upon a variety of factors, including, for example, the polarity of the involved molecules and the charge (e.g., positive or negative), if any, of the involved molecules. Non-covalent associations are selected from electrostatics (e.g., ion-ion, ion-dipole, and dipole-dipole), hydrogen bonds, π-π stacking interactions, van der Waal's forces, hydrophobic and solvatophobic effects, and the like, and combinations thereof.

Generally, “the patient” or “a patient” refers to animals, including vertebrates, preferably mammals, and most preferably humans.

A “peptosome” generally refers to a vesicle which is assembled from copolypeptides in aqueous or near-aqueous solutions. Peptosomes are composed substantially of amino acid residues or modified amino acids of either natural, synthetic, or semi-synthetic origin. The term “substantially” means that greater than approximately 50 mole percent (%) of the vesicle components are composed of amino acids or modified amino acid residues. If desired, greater than approximately 60%, 70%, 80%, 90%, 95%, or even 100 mole % of the peptosome components are composed of amino acid or modified amino acid residues. Peptosomes may be, for example, supramolecular complexes, stabilized, or otherwise crosslinked.

“Photopolymerize” and “Photopolymerization” refer to a technique wherein light is used to initiate and propagate a polymerization reaction to form a linear or cross-linked polymer structure. In the context of embodiments of the present invention, this type of system utilizes, for example, a light source, photoinitiators, and photocrosslinkable biopolymer/biodendritic macromolecular structure, including dendritic supramolecular complexes. Photopolymerization can occur via a single- or multi-photon process. In two-photon polymerization, laser excitation of a photoinitiator proceeds through at least one virtual or nonstationary state. The photo-initiator will absorb two near-IR photons, driving it into the S₂ state, followed by decay to the S₁, and intersystem crossing to the long-lived triplet state. When the spatial density of the incident photons is high, the initiator molecule (i.e., in the triplet state) will abstract an electron from, for example, triethylamine (TEA), and thus start the photocrosslinking reaction of the polymer. Indeed, controlled microfabrication via, for example, 2-photon-induced polymerication (TRIP) has been used to develop a variety of biomedical-related polymeric materials.

“Piezoelectric” refers to systems driven by the effect of certain crystals such as lead-zirconate-titanate, and other materials, which expand and contract in an alternating (i.e., charged) electrical field.

“Polymer” or “polymeric” refers to a substance composed of molecules which have long sequences of one or more species of atoms, or groups of atoms, linked to each other by primary, usually covalent bonds. Thus, polymers are molecules formed from the chemical union of two or more repeating units. Accordingly, included within the term “polymer” may be, for example, dimers, trimers, and oligomers. The polymer may be synthetic, naturally occurring, or semisynthetic, and linear, networked, or branched. In a preferred form, “polymer” refers to molecules which comprise 10 or more repeating units. In addition, a “polymer” can be synthesized by starting from a mixture of monomers followed by a polymerization reaction, and subsequently functionalized by coupling with suitable compounds or groups. The term “polymer” may also refer to compositions comprising block copolymers or terpolymers, random copolymers or terpolymers, random copolymers, polymeric networks, branched polymers and copolymers, hyperbranched polymers and copolymers, dendritic polymers and copolymers, hydrogels, and the like, all of which may also be grafted and mixtures thereof.

A “polymersome” generally refers to a vesicle which is assembled from polymers or copolymers in aqueous solutions. Polymersomes are composed substantially of synthetic polymers and/or copolymers. Unlike liposomes, a polymersome does not include lipids or phospholipids as its majority component. Consequently, polymersomes can be acoustically, thermally, mechanically, and chemically distinct and, in particular, more durable and resilient than the most stable of lipid vesicles. Polymersomes assemble during processes of lamellar swelling (e.g., by film or bulk rehydration), through an additional phoresis step, or by other known methods. Like liposomes, polymersomes form by “self-assembly,” a spontaneous, entropy-driven process of preparing a closed semi-permeable membrane. The choice of synthetic polymers, as well as the choice of molecular weight of the polymer, are important, as these distinctive molecular features impart polymersomes with a broad range of tunable carrier properties. The term “substantially” means that greater than 50 mole percent (%) of the vesicle components are composed of synthetic polymers. If desired, greater than approximately 60%, 70%, 80%, 90%, 95%, or even 100 mole % of the polymersome components are composed of synthetic polymers. Polymersomes may be, for example, supramolecular complexes, stabilized, or otherwise cross-linked.

A “polypeptide” generally refers to a single linear chain of amino acids, and the family of short molecules formed from the linking, in a defined order, of various α-amino acids. The link between one amino acid residue and the next is an amide bond, and is sometimes referred to as a peptide bond. Polypeptides do not possess a defined tertiary or quaternary structure.

A “precursor” to a targeting ligand refers to any material or substance which may be converted to a targeting ligand. Such conversion may involve, for example, anchoring a precursor to a targeting ligand. Exemplary targeting precursor moieties include maleimide groups, disulfide groups such as ortho-pyridyl disulfide; vinylsulfone groups; azide groups; α-iodo acetyl groups; and the like.

A “protein” generally refers to molecules comprising, and preferably consisting essentially of α-amino acids in peptide linkages. Included within the term “protein” are globular proteins such as, for example, albumins, globulins, histones, and fibrous proteins such as collagens, elastins, and keratins. Also included within the term “protein” are “compound proteins,” wherein a protein molecule is united with a nonprotein molecule such as, for example, nucleoproteins, mucoproteins, lipoproteins, and metalloproteins. The proteins may be naturally occurring, synthetic, or semi-synthetic.

“Receptor” generally refers to a molecular structure within a cell, or on the surface of a cell, which is generally characterized by the selective binding of a specific substance. Exemplary receptors include, for example, cell-surface receptors for peptide hormones, neurotransmitters, antigens, complement fragments, immunoglobulins, cytoplasmic receptors for steroid hormones, and the like.

“Region of a patient” or “region of the patient” refers to a particular area or portion of the patient and, in some instances, to regions throughout the entire patient. Exemplary of such regions are the eye; gastrointestinal region; the cardiovascular region, including myocardial tissue; the renal region as well as other bodily regions, tissues, lymphocytes, receptors, organs, and the like, including the vasculature and circulatory system; as well as diseased tissue, including cancerous tissue such as in the prostate or breast.

“Region of a patient” includes, for example, regions to be imaged with diagnostic imaging, regions to be treated with a bioactive agent (e.g., a therapeutic), regions to be targeted for the delivery of a bioactive agent, and regions of elevated temperature. The “region of a patient” is preferably internal; although, if desired, it may be external. The term “vasculature” denotes blood vessels including arteries, veins, and the like. The phrase “gastrointestinal region” includes the region defined by the esophagus, stomach, small and large intestines, and rectum. The phrase “renal region” denotes the region defined by the kidney and the vasculature that leads directly to and from the kidney, and it includes the abdominal aorta. “Region to be targeted,” “targeted region,” “target region,” or “target” refers to a region of the patient where delivery of a therapeutic is desired. “Region to be imaged” or “imaging region” denotes a region of a patient where diagnostic imaging is desired.

“Resuspending” refers to adding a liquid to change a dried physical state of a substance to a liquid physical state. For example, a dried therapeutic delivery system may be resuspended in a liquid such that it has similar characteristics in the dried and resuspended states. The liquid may be an aqueous liquid or an organic liquid, for example. In addition, the resuspending medium may be a cryopreservative. Polyethylene glycol, sucrose, glucose, fructose, mannose, trebalose, glycerol, propylene glycol, sodium chloride, and the like; may be useful as a resuspending medium.

“Rupture” refers to the act of breaking, bursting, or disassociating, usually in response to specific stimuli such as, for example, ultrasonic energy; “rupturing” means undergoing rupture.

“Sonolysis” refers to the disruption of biological cells, either directly or indirectly, through application of ultrasonic energy.

“Spray drying” refers to drying by bringing an emulsion of surfactant and a therapeutic, or portions thereof, in the form of a spray into contact with a gas such as air, and recovering it in the form of a dried emulsion. A blowing agent (e.g., methylene chloride) may be stabilized by said surfactant.

“Stabilized” or “stabilization” refers to exposure of materials such as polymers, mixtures, emulsions, and the like, including materials of embodiments of the present invention, to stabilizing materials or stabilizing compounds. “Stabilizing material” or “stabilizing compound” refers to any material which is capable of improving the stability of compositions containing the therapeutics for use with embodiments of the present invention, including targeting ligands and/or other bioactive agents described herein, and including, for example, mixtures, suspensions, emulsions, dispersions, vesicles, and the like. Encompassed in the definition of “stabilizing material” are certain bioactive agents. The improved stability involves, for example, the maintenance of a relatively balanced condition, and may be exemplified, for example, by increased resistance of the composition against destruction, decomposition, degradation, rupture, and the like. In the case of preferred embodiments involving nanocarriers filled with therapeutics and/or bioactive agents, the stabilizing compounds may serve to either form the vesicles or stabilize the vesicles, in either way serving to minimize or substantially including completely preventing the escape of liquids, therapeutics, and/or bioactive agents from the vesicles, until said release is desired. The term “substantially,” as used in the present context of preventing escape of liquids, therapeutics and/or bioactive agents from said nanocarriers, means greater than approximately 50% is maintained entrapped in the nanocarriers until release is desired, and preferably greater than approximately 60%, more preferably greater than approximately 70%, even more preferably greater than approximately 80%, still even more preferably greater than approximately 90%; is maintained entrapped in the nanocarriers until release is desired. In particularly preferred embodiments, greater than approximately 95% of the liquids, therapeutics, and/or bioactive agents maintained entrapped until release is desired. The liquids, therapeutics or bioactive agents may also be completely entrapped (i.e., approximately 100% is maintained entrapped) until release is desired. Exemplary stabilizing materials include, for example, lipids, proteins, polymers, carbohydrates, surfactants, and the like. The resulting mixture, suspension, emulsion, or the like, may comprise walls (i.e., films, membranes, and the like) around the bioactive agent, or may be substantially devoid of walls or membranes, if desired. The stabilizing may, if desired, form droplets. The stabilizing material may also comprise salts and/or sugars. In certain embodiments, the stabilizing materials may be substantially (i.e., including completely) cross-linked. The stabilizing material may be neutral, positively charged, or negatively charged.

“Supramolecular assembly,” “supramolecular complex,” or “supramolecular structure” generally refers to a defined complex of molecules held together by non-covalent bonds and, in several preferred embodiments, is designed for a specified level of acoustic sensitivity. While a supramolecular complex can be simply composed of two molecules (e.g., a DNA double helix or an inclusion compound), in the embodiments of the present invention, supramolecular complex refers to larger complexes of molecules that form sphere, rod-like, and/or other vesicles for the delivery of therapeutics or other substances to the patient. The dimensions of supramolecular complexes can range from nanometers to micrometers. Supramolecular complexes allow access to nanoscale objects using a bottom-up approach, in much fewer steps than a single molecule of similar dimensions. The process by which a supramolecular complex forms is termed “self-assembly” or “self-organization,” where self-assembly is the process by which individual molecules form the defined aggregate, and self-organization is the process by which those aggregates create higher-order structures. A great advantage to the supramolecular approach in drug delivery is that the larger complexes of molecules will disassociate or degrade back into the individual molecules comprising said assembly, which can be broken down by the patient. Some of the preferred embodiments of the present invention are, for example, supramolecular structures formed from, for example, block copolymers.

“Surfactant” or “surface active agent” refers to a substance that alters energy relationships at interfaces such as, for example, synthetic organic compounds displaying surface activity including inter alia, wetting agents, detergents, penetrants, spreaders, dispersing agents, and foaming agents. The term surfactant is derived from “surface active agent;” surfactants are often organic compounds that are amphipathic and typically are classified into three primary groups: (1) anionic, (2) cationic, and (3) non-ionic.

A “suspension” or “dispersion” refers to a mixture, preferably finely divided, of two or more phases (i.e., solid, liquid or gas) such as, for example, liquid in liquid, solid in solid, gas in liquid, and the like, which preferably can remain stable for extended periods of time.

“Synthetic polymer” refers to a polymer that comprises, in whole or in part, substances that are created by chemical synthesis, rather than produced naturally by an organism. A substance that is a naturally occurring polymer may also be created by chemical synthesis (i.e., peptides and nucleotides can be created either naturally or in the laboratory).

“Targeted vesicle” refers to a vesicle such as, for example, a nanocarrier with a targeting ligand covalently or noncovalently attached to, or anchored within, said vesicle. “Targeting ligand” or “targeting moiety” generally refers to any material or substance which may promote targeting of tissues and/or receptors, in vivo or in vitro, with the compositions of embodiments of the present invention. The targeting ligand may be synthetic, semi-synthetic, or naturally occurring. Materials or substances which may serve as targeting ligands include, for example, proteins, including antibodies, antibody fragments, hormones, hormone analogues, glycoproteins and lectins, peptides, and polypeptides; amino acids; sugars; saccharides including monosaccharides and polysaccharides; carbohydrates; vitamins; steroids; steroid analogs; hormones; cofactors; bioactive agents; and genetic material including aptamers, nucleosides, nucleotides, nucleotide acid constructs, and polynucleotides. Compositions facilitating magnetic delivery, which may be hereafter referred to as “magnetic compositions” are also preferred targeting moieties for use with embodiments of the present invention, and may be used alone or in combination with other targeting moieties such as, for example, ligands of synthetic, semi-synthetic, or naturally occurring origin.

“Therapeutic,” “drug,” “pharmaceutical” “pharmacologically active agent,” “permeant,” or “deliverable substance” refers to any pharmaceutical, drug, or prophylactic agent which may be used in the treatment, including the prevention, diagnosis, alleviation, or cure of a malady, affliction, disease, or injury of the patient. In addition, “therapeutic” means any chemical or biological material or compound suitable for delivery by the methods previously known in the art, combined with the methods of and/or by the present invention, which induces a desired effect such as a biological or pharmacological effect, which may include, but is not limited to (1) having a prophylactic effect on the patient, and preventing an undesired biological effect such as, for example, preventing an infection; (2) alleviating a condition caused by a disease, for example, alleviating pain or inflammation caused as a result of disease; (3) either alleviating, reducing, or completely eliminating the disease from the patient; and/or (4) the placement within the viable tissue layers of the patient of a compound or formulation which can react, optionally in a reversible manner, to changes in the concentration of a particular analyte and, in so doing, cause a detectable shift in this compound or formulation's measurable response to the application of, for example, energy to this area which may be electromagnetic, mechanical, or most preferably acoustic (i.e., ultrasonic). The effect may be local such as providing for local tissue permeability to, for example, a nucleic acid (e.g., RNAi, siRNA, etc.) or the effect may be systemic. The term “therapeutic” also includes contrast agents and dyes for visualization. Obviously, therapeutically useful peptides, polypeptides, polynucleotides, and other therapeutic macromolecules may also be included within the meaning of the term “Pharmaceutical” “drug,” or “therapeutic.”

“Therapeutic macromolecule” refers to a pharmacologically active agent produced either partially, or in full, by modern biotechnological and/or other techniques (e.g., proteins, nucleic acids, and synthetic peptides).

“Therapeutic ultrasound” refers to high-intensity focused ultrasound, or HIFU.

“Therapy” refers to the treatment of a disease or disorder by various methods.

“Tissue” refers generally to specialized cells which may perform a particular function. The term “tissue” may refer to an individual cell, or a plurality or aggregate of cells (e.g., membranes, blood, or organs). The term “tissue” also includes reference to an abnormal cell or a plurality of abnormal cells. Exemplary tissues include myocardial tissue including myocardial cells and cardiomyocytes; membranous tissues including endothelium and epithelium; laminae and connective tissue, including interstitial tissue; and tumors.

“Transdermal,” “percutaneous,” “transmembrane,” “transmucosal,” or “transbuccal” refers to passage of a permeant such as a therapeutic, into or through the biological membrane or tissue to achieve effective therapeutic levels of a drug in, for example, blood, tissue, and/or cells, or the passage of a molecule present in the body (“analyte”) out through the biological membrane or tissue, so that the analyte molecule may be collected on the outside of the body. Disruption of said biological membrane, by the methodologies of this disclosure, may preferably facilitate said passage of said permeant.

“Ultrasonic” refers to frequencies of sound above normal human hearing, generally accepted to be at 20 KHz to 2 MHZ and above, but also extended down to the 5 KHz to 20 KHz range in certain processing applications. Subsonic, supersonic, or transsonic has to do with the speed of sound. As used throughout this specification, “ultrasonic” also refers to any processes, practices, or methods employing ultrasound, either high-intensity focused ultrasound (HIFU), for therapeutic or other purposes, or diagnostic ultrasound, for imaging; or any other use of acoustic energy.

“Ultrasonically sensitive material” or “ultrasonically sensitive materials” is generally used herein to refer to a compound, molecule, drug, therapeutic, polymer, copolymer, and/or other material, including those of synthetic, semi-synthetic, or natural origin; alone or in combination with other materials that may, or may not be ultrasonically sensitive; which are sensitive to mechanical rectification or other aspects of exposure to high-intensity ultrasound, high-intensity focused ultrasound (HIFU), or ultrasound (i.e., said material changes shape, conformation, and/or chemical reactivity, etc., in response to ultrasound). These ultrasonically sensitive materials can be used in various applications of the present teachings, and several types of ultrasonically sensitive material may be employed, either alone or in combination, with other compounds. A most preferred embodiment is the disassociation of therapeutic-containing nanocarriers of embodiments of the present invention by ultrasound, at a treatment site of the patient, releasing said therapeutic(s). Various other embodiments of ultrasonically sensitive materials include pharmaceutical agents complexed with ultrasonically sensitive materials, either covalently or through non-covalent interactions. For example, ultrasonically sensitive materials can be switchable to release a pharmaceutical agent. In some embodiments, molecules sensitive to asymmetrical waveforms prevalent due to nonlinear propagation of ultrasonic waveforms may be used. With such waveforms, the peak positive pressure can be an order of magnitude, or more, greater than the peak negative pressure. Further, a compressible material, or part of a material, can act as an effecter by changing its shape considerably during ultrasound exposure, thus triggering a specific event or process, including drug release, formation of a gaseous contrast microbubble for imaging, enhancing acoustic cavitation, etc. In addition, “ultrasonically sensitive materials” can enhance chemical reactivity, thereby having a direct pharmacological effect, or said materials can enhance the pharmacological effect of other therapeutics and/or prodrugs. Likewise, ultrasonically sensitive materials include molecules that are sensitive to peak negative or positive pressures and/or ultrasonic intensities.

Additional embodiments of “ultrasonically sensitive materials” include use of molecules, polymers, therapeutics, and the like, that are sensitive to free radical concentration. For example, acoustic cavitation can generate free radicals that may be used as a trigger, causing the molecules to become effecters. Moreover, since free radicals are part of the natural inflammation process, such free radical sensitive molecules can be useful effecters even without an ultrasound trigger, thus allowing more pharmacological control of the inflammation process. These free radical detecting molecules can also be used for cavitation detection in vivo as inflammation detectors. Further, the term “ultrasonically sensitive materials” includes molecules designed to generate or process dissolved gasses so as to form free gas bubbles in response to many different triggering events or sensing environments. For example, when bound to a tumor-specific antigen, the molecule can change functionality and produce a gas bubble. This gas bubble would then be useful as a contrast agent for diagnostic detection or as a nucleus for acoustic cavitation. First, cardiac infarction or stroke produces ischemic tissue and/or inflammation which, in turn, damages affected tissues by free radical formation. A free radical sensitive molecule can release drugs comprised of contrast agents, thereby allowing quicker diagnosis and/or treatment. Second, a molecule reacting to some aspect of an ultrasonic exposure (e.g., pressure, intensity, cavitation asymmetric waveforms due to nonlinear propagation, cavitation, and/or free radical formation due to cavitation) can be an ideal candidate as a drug carrier, contrast agent delivery vehicle, nuclei for therapeutic cavitation, etc. Third, ultrasonically sensitive molecules that change in response to ultrasound exposure, by any of the mechanisms mentioned herein, can have biological effectiveness by many different mechanisms, including switchable enzymatic activity; switchable water affinity (e.g., change from hydrophobic to hydrophilic); switchable buffer modulating local pH; switchable chemical reactivity allowing remote ultrasound control of an in vivo chemical reaction, perhaps, for example, producing a drug in situ or modulating drug activity; and/or switchable conformations of a smart molecule, allowing the covering or uncovering (i.e., presentation) of an active site which could bind with any designed binding specificity (e.g., a drug which was inactive [inert] until triggered locally by ultrasound). Fourth, ultrasonically sensitive molecules that are switchable free radical scavengers can be activated by ultrasound for tissue protection, for example, following a stroke or cardiac infarction.

Other embodiments of “ultrasonically sensitive materials” can be directly or indirectly affected by free radical generators and scavengers as cavitation modulators. Additional “ultrasonically sensitive materials” can work with changes in localized concentration of many other reagents, molecules, drugs, etc., to protect some regions of the patient and to predispose others to, for example, penetration of biological barriers by a variety of molecules, compounds, or other structures. Exemplary applications include modulating cavitation nuclei, either naturally or by some ultrasonically sensitive molecules designed to act as cavitation nuclei or a processor of cavitation nuclei and which are controlled in their activity by ultrasonically induced changes in free radical concentrations, pH, etc.

“Vacuum drying” refers to drying under reduced air pressure, resulting in drying at a lower temperature than required at full pressure.

“Van der Waal's forces” refers to dispersion forces between non-polar molecules that are accounted for by quantum mechanics. Van der Waal's forces are generally associated with momentary dipole moments which are induced by neighboring molecules, involving changes in electron distribution.

“Vector” and “cloning vehicle” generally refers to non-chromosomal double-stranded DNA comprising an intact replicon such that the vector is replicated when placed within a unicellular organism (e.g., a bacterium), for example, by a process of transformation.

“Viral vectors” include retroviruses, adenoviruses, herpesvirus, papovirus, or otherwise modified naturally occurring viruses. Vector also means a formulation of DNA, with a chemical or substance, which allows uptake by cells. In addition, materials could be delivered to inhibit the expression of a gene. Approaches include antisense agents such as synthetic oligonucleotides which are complementary to RNA or the use of plasmids expressing the reverse complement of a gene; catalytic RNAs or ribozymes which can specifically degrade RNA sequences by preparing mutant transcripts lacking a domain for activation; or over-expressed recombinant proteins, which antagonize the expression, or function, of other activities. Advances in biochemistry and molecular biology in recent years have led to the construction of recombinant vectors in which, for example, retroviruses and plasmids are made to contain exogenous RNA, or DNA, respectively. In particular instances, the recombinant vector can include heterologous RNA or DNA, by which is meant RNA or DNA which codes for a polypeptide not produced by the organism (e.g., the patient) susceptible to transformation by the recombinant vector. The production of recombinant RNA and DNA vectors is well understood in the prior art, and need not be summarized here.

“Vesicle” generally refers to an entity which is usually characterized by the presence of one or more walls or membranes which form one or more internal voids. Vesicles such as the nanocarriers of embodiments of the present invention, may be formulated, for example, from a stabilizing material such as a copolymer, including the various polymers described herein, especially “block copolymers,” a proteinaceous material, including the various polypeptides described herein, and a lipid. As discussed herein, vesicles may also be formulated from carbohydrates, surfactants, and other stabilizing materials, as desired. The proteins, polymers, copolymers, and/or other vesicle-forming materials may be natural, synthetic, or semi-synthetic. Preferred vesicles are those which comprise walls or membranes formulated from polymers, dendritic polymers, copolymers, polypeptides, copolypeptides, etc. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle comprised of one monolayer or bilayer, an oligolamellar vesicle comprised of approximately two or three monolayers or bilayers, or a multilamellar vesicle comprised of more than approximately three monolayers or bilayers. The walls or membranes of vesicles may be substantially solid (i.e., uniform), or they may be porous or semi-porous. The vesicles described herein include such entities commonly referred to as, for example, (1) microspheres, (2) hydrogels, (3) microcapsules, (4) microbubbles, (5) particles, (6) nanocarriers, (7) nanoparticles, (8) nanovesicles, (9) micelles, (10) bubbles, (11) microbubbles, (12) polymer-coated bubbles and/or protein-coated bubbles, (13) polymer matrixes, (14) microbubbles and/or microspheres, (15) nanospheres, (16) microballoons, (17) aerogels, (18) clathrate-bound vesicles, and (19) the like. The internal void of the vesicles may be filled with a wide variety of materials including, for example, (1) water, (2) oil, (3) liquids, (4) therapeutics, and (5) bioactive agents, and/or (6) other materials. The vesicles may also comprise one or more targeting moieties, if desired.

“Vesicle stability” refers to the ability of vesicles to retain the therapeutic or bioactive agents entrapped therein, after being exposed, for approximately one minute, to a pressure of approximately 100 millimeters (mm) of mercury (Hg). Vesicle stability is measured in percent (%), this being the fraction of the amount of gas which is originally entrapped in the vesicle and which is retained after release of the pressure. Vesicle stability also includes “vesicle resilience,” which is the ability of a vesicle to return to its original size after the release of said pressure.

The following is a detailed description of illustrative embodiments of the present invention. As these embodiments are described with reference to the aforementioned drawings and definitions, various modifications or adaptations of the methods and or specific structures described herein may become apparent to those skilled in the art. All such modifications, adaptations, or variations that rely on the teachings of this disclosure, and through which these teachings have advanced the art, are considered to be within the spirit and scope of this specification.

### OVERVIEW OF THE PREFERRED EMBODIMENTS

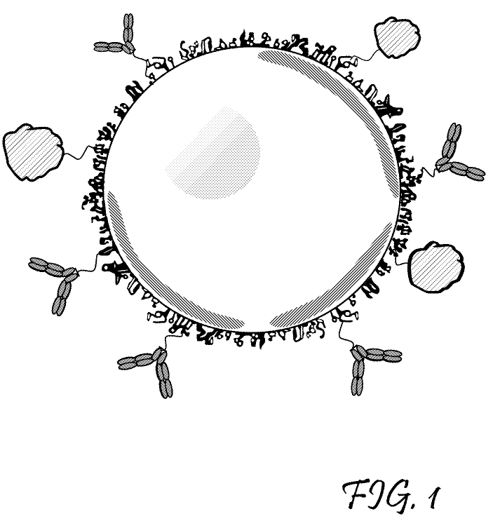

Therapeutic macromolecules such as, for example, antisense oligonucleotides, small interfering RNA (siRNA), and plasmid DNA show enormous potential in the treatment of for example, a wide variety of inherited and acquired genetic disorders, viral infections, and cancer. Gene therapy aims to deliver these nucleic acids to specific cells to, for example, introduce novel genes and/or repair malfunctioning ones. However, delivery of said genetic and other therapeutic materials to cells, most with the requirement of intracellular delivery, provides multiple challenges which many preferred embodiments of the present invention are designed to overcome (FIG. 1).

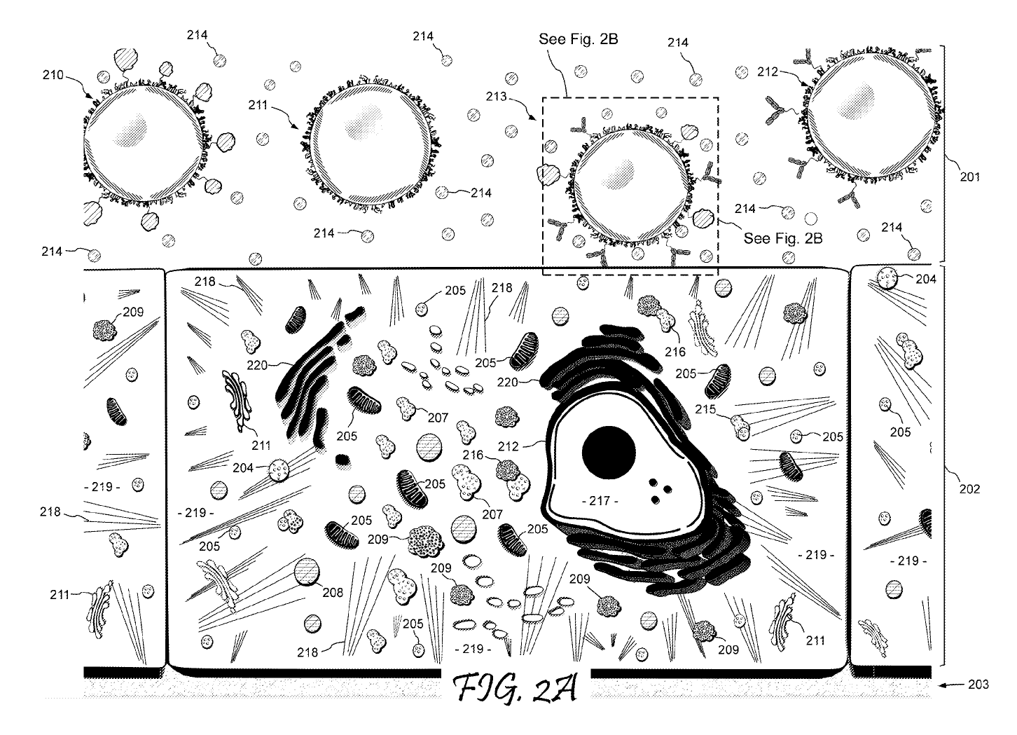

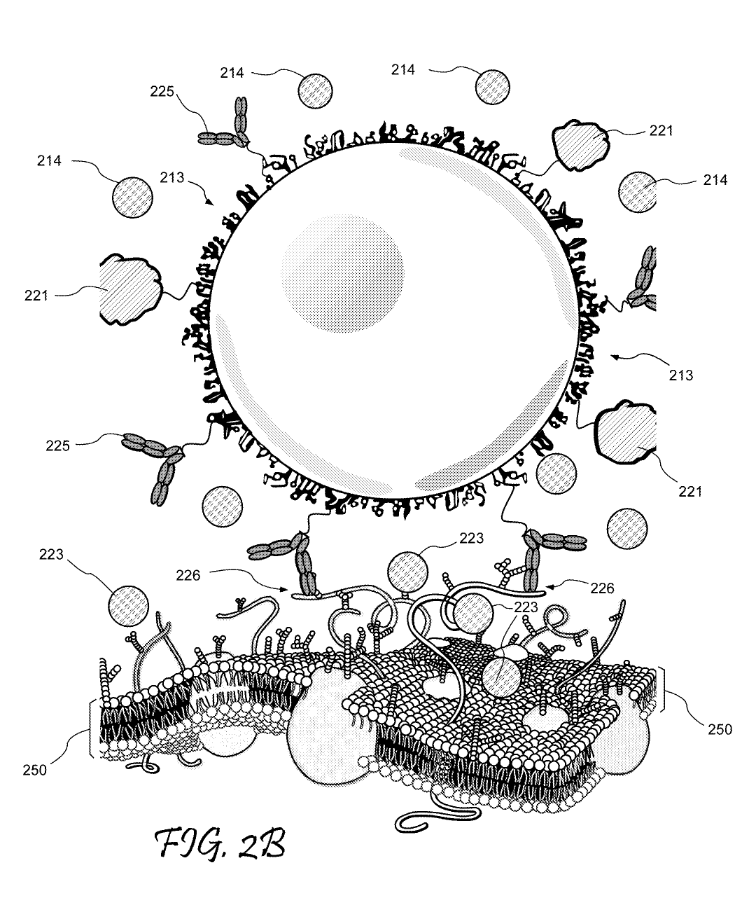

As discussed herein, to exert efficiently its activity without toxic effects, a drug must reach its pharmacological site(s) of action within the body. This may be inside the cell cytoplasm (FIG. 2A [219]) or into the nucleus (217) or other specific organelles such as lysosomes (209), mitochondria (205), golgi apparatus (206), and/or the endoplasmic reticulum (220). Example pharmaceuticals requiring intracellular delivery include preparations for gene, antisense, and other therapeutic approaches, many of which must reach the cell nuclei 217; proapoptotic drugs, which target mitochondria 205; lysosomal enzymes which have to reach the lysosomal compartments 209; and many others. Thus, the intracellular transport of different biologically active molecules and macromolecules is currently one of the key problems in drug delivery.

As will be reviewed in greater detail below, intracellular membrane barriers exist both due to the cell membrane itself (FIGS. 2A and 2B [250]) and a variety of membrane-bounded intracellular vesicles (FIG. 2A); including for some therapeutics, the nuclear membrane (212). In addition, the cytoplasm may constitute a significant diffusional barrier to gene transfer to the nucleus, depending primarily on therapeutic size. Embodiments of the present invention represent many new materials, methods, and strategies to overcome these biological barriers and other challenges so troublesome to efficient intracellular drug delivery, especially for the delivery of therapeutic macromolecules.

FIG. 2A illustrates a section of a continuous blood vessel endothelium (202) of a patient. Following parenteral administration, depending on their size, surface charge, hydrophobicity and hydrophilicity, and other variables, the drug-carrying nanocarriers of this disclosure will be present at specific concentrations in the blood vessel lumen (201), along with, in this example, co-administered gaseous contrast agents (microbubbles; 214). In FIG. 2A, various embodiments of the therapeutic-carrying nanocarriers of this specification are illustrated (211-213). Nanocarriers composed substantially of, for example, supramolecular complexes, may be labeled with targeting moieties such as magnetic particles (210), unlabeled (211), or labelled with ligands such as antibody or antibody fragments (212), other ligands, or a combination of targeting moieties (213).

FIG. 2B illustrates a magnified view of a small portion of FIG. 2A. A fully assembled, therapeutic-containing nanocarrier is illustrated in FIG. 2B, where said nanocarrier (213) is in close proximity to the plasma membrane (250) of a target cell/tissue. In this embodiment FIG. 2B, targeting ligands (i.e., antibody fragments [225] and magnetic particles [221]) are attached to said nanocarrier, again in this example, using tethers comprised of, for example, polyethylene glycol, where said ligands have actively guided the vesicle to its target. In additional embodiments, nanocarriers may be passively targeted. Contrast agents are also present (214) surrounding the nanocarrier, and in this example, filling the extracellular spaces surrounding target tissues. Said microbubbles are at predefined concentrations so they may be, in some embodiments, at high densities (223) close to target cell membranes (250) and otherwise FIG. 2B. In additional embodiments, not illustrated, contrast agents (214) may be labeled with one or more targeting ligands, which may or may not be attached to said contrast agents via tethers. Other preferred embodiments, again not illustrated, include enclosing said therapeutic-containing nanocarriers, either alone or combined with free therapeutic(s), in an acoustically responsive or other drug-delivery polymer matrix such as a hydrogel.

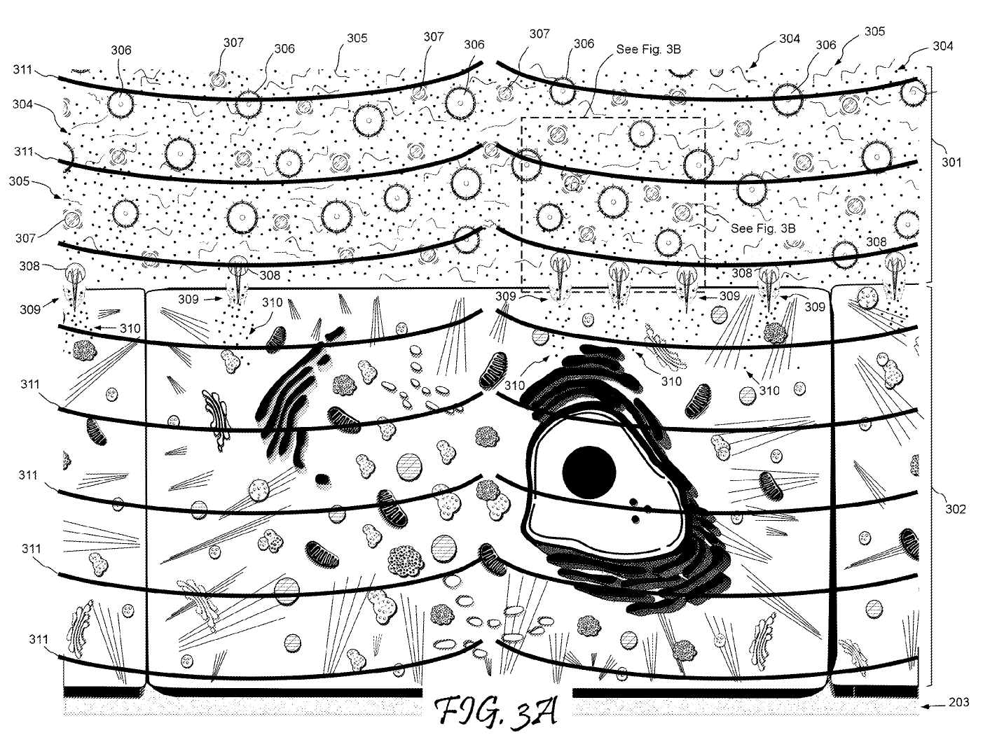

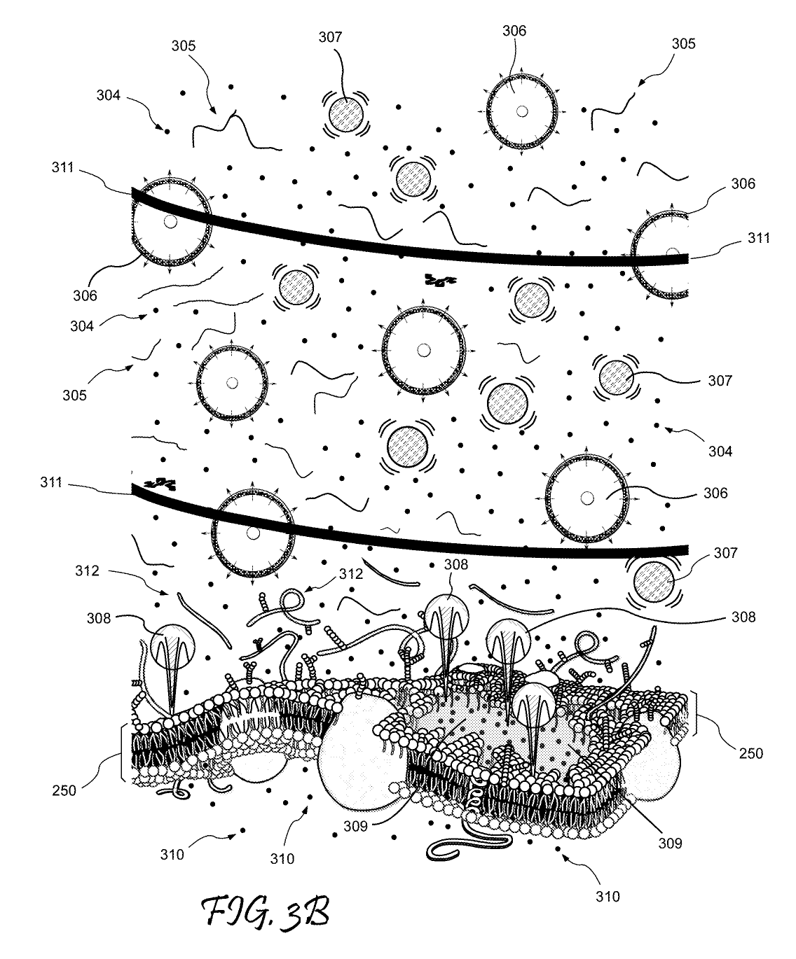

Once the therapeutic-containing nanocarriers have reached their target, therapeutic release is initiated by exposure of the target region to, in this example, pulsed, high-intensity focused ultrasonic energy (FIGS. 3A and 3B [311]). Without wishing to be bound by any particular theory, when an ultrasound wave propagates in tissue, a mechanical strain is induced, where strain refers to the relative change in dimensions or shape of the material that is subjected to stress, and where said strain may be especially significant near gas or vapor bubbles. Depending on a variety of parameters, acoustic cavitation may result, a most important phenomenon for the application of the present teachings. Cavitation, in a broad sense, refers to ultrasonically induced activity occurring in a liquid or liquid-like material that contains bubbles or pockets containing gas or vapor. These bubbles originate at locations termed “nucleation sites,” the exact nature and source of which are not well understood in a complex medium such as tissue. Or, as in preferred embodiments of this disclosure, these bubbles may also be introduced into the insonated area (214), either directly or indirectly, in the form of, for example, gaseous contrast agents (i.e., microbubbles) (214). Under ultrasonic stimulation (311) with the appropriate parameters (e.g., focus, frequency, pulse length, pulse repetition frequency, etc.), said bubbles oscillate (307), creating a circulating fluid flow—called microstreaming—around the bubble, with velocities and shear rates proportional to the amplitude of the oscillation. At high amplitudes, the associated shear forces are capable of shearing open cells and synthetic vesicles such as those of this disclosure. Further, said bubbles may collapse, sending out, for example, shock waves in their immediate vicinity (306). These shock waves and other disturbances at the target site also result in nanocarrier disruption (305) and therapeutic release (304). In addition, bubbles close to surfaces while undergoing inertial cavitation (308), in an optimal embodiment, may emit membrane-piercing microjets (308). If properly controlled, said microjets (308) preferably function in the present disclosure, for example, in permeabilizing cell membranes at the target, and in some cases tearing pieces of membrane (309) from target cells and tissues. Emitted shock waves (306) from collapsing bubbles probably also contribute to membrane disruption. Thus, cavitation is a potentially violent event, effectively concentrating ultrasonic energy into a small volume.

Said oscillating bubbles can also result in acoustic pressure, a net force acting on other suspended bodies in the vicinity of an oscillating bubble. If the body is more dense than the suspending liquid, the body is pushed toward the oscillating bubble; if less dense, the body is repelled (Nyborg, 2001). Most of the drug-carrying vesicles of this disclosure are more dense than water, and thus will be convected toward the bubble, thus increasing the dispersive transport of the drug carrier, particularly if the vesicle is drawn into the microstreaming field around said bubble and is sheared open by the high shear rate, thus releasing therapeutic. If the vesicle is another microbubble, it will be dispersed away from the primary oscillating bubble because it is less dense. Thus, a field of microbubbles such as those co-administered with the nanocarriers described herein, will tend to spread itself in the ultrasonic field, and at the same time, attract and shear more dense vesicles such as suspended cells or the nanocarriers of the present teachings. In theory, said nanocarriers will not be acoustically active since they contain no gas. However, these nanocarriers should be drawn toward and then sheared open by the action of the surrounding cavitating bubbles, as long as said bubbles are at sufficient densities.

In addition to the many mechanical stresses summarized above, cavitation may affect a biological system by virtue of a temperature increase and/or free radical production. While cavitation can produce extremely high temperatures immediately close to the nucleation site, it is traditionally referred to as a non-thermal mechanism of tissue damage (O'Brien, 2007). Indeed, for the purposes of this specification, temperature increases at the target must be minimized. This is accomplished, in part, by selecting the most appropriate ultrasound parameters for a given application. The occurrence of cavitation, and its behavior, depends on many variables, including the ultrasonic pressure, whether the ultrasonic field is focused or unfocused, continuous or pulsed, or combinations thereof; to what degree there are standing waves (i.e., energy reflecting back onto itself); the nature and state of the material and its boundaries; as well as many other variables. Thus, the ultrasonic energy utilized by the present teachings must have its properties tailored to specific drug delivery applications. The major goal in many of said applications is to control the amount and extent of acoustic cavitation at the target site. If properly controlled, the nanocarriers of this disclosure will be effectively disrupted (305) and the enclosed therapeutic(s) freed into the surrounding medium (304). Said cavitation activity also results in tissue permeation, allowing extravasation and therapeutic entry, importantly in the case of therapeutic nucleic acids, avoiding entry by the usually destructive endocytic pathway, described in detail below, while minimizing permanent and long-term damage (i.e., sonolysis and cytotoxicity) to the patient.

Because many eucaryotic cells inhabit mechanically stressful environments, their plasma membranes are frequently disrupted. Survival requires that the cell rapidly repair or reseal said disruption (McNeil et al., 2003). Obviously, this phenomena is critical for the successful application of the present teachings. Rapid membrane resealing is an active and complex structural modification that employs endomembrane as its primary building block (i.e., literally a “patch”), and cytoskeletal and membrane fusion proteins as its catalysts. Endomembrane is delivered to the damaged plasma membrane through exocytosis, a ubiquitous Ca²⁺-triggered response to disruption. Tissue and cell level architecture may prevent disruptions from occurring, either by shielding cells from damaging levels of force or, when this is not possible, by promoting safe force transmission through the plasma membrane via protein-based cables and linkages (McNeil et al., 2003). Therefore, membrane damage and its subsequent repair is a normal process occurring in the patient; embodiments of the present invention take advantage of these repair mechanisms for assisting in safe and effective intracellular drug delivery.

Without wishing to be bound by any particular theory, in order to more fully illustrate the novelty and importance of the present teachings, some of the preferred embodiments of this specification will be discussed in the context of currently accepted biological structures and how said embodiments may overcome barriers to conventional drug delivery known in the art. These biological barriers can be broadly categorized into extracellular and intracellular barriers.

Extracellular barriers. To protect, for example, therapeutic nucleic acids from degradation while transversing extracellular spaces before reaching their target, said therapeutics are enclosed within the nanocarriers of the present teachings, comprised preferably of biodegradable polymers, or mixtures thereof where said nanocarriers are composed substantially of supramolecular complexes. Said vesicles must travel through extracellular barriers such as blood, before they reach their target tissue, which may be situated near, as well as far away from, the site of administration. Systemic, parenteral administration of the nanocarriers of this disclosure is strongly preferred, as it may allow the distribution of said carriers via the bloodstream to tissues that are otherwise difficult to reach via more localized application. However, the blood forms a major barrier to said vesicles as biomolecules (e.g., albumin) are known to extensively bind to cationic carriers, causing a neutralization or reversion of their surface change. Neutralization of said nanocarriers by albumin or other biomolecules abolishes the electrostatic repulsion that exists between said carriers, allowing them to come into close contact upon collision. When such close contact happens, for example, Van der Walls forces may take place and hold the vesicles together, resulting in aggregates. In addition, the binding of such negatively charged biomacromolecules to, in an optimal embodiment, self-assembled, acoustically responsive nanocarriers, may subsequently deassemble said vesicles. These difficulties and complications will be largely dependent on nanocarrier composition and solved in a variety of ways, some of which are described herein.

Other major extracellular barriers that must be overcome include endothelial cells and basement membranes. Indeed, the nanocarriers of this disclosure have to extravasate before they can reach tissues localized outside of the bloodstream. With conventional drug-containing vesicles, extravasation is mainly determined by the vesicle's size and the permeability of the capillary walls, a characteristic that greatly varies between tissues (Takakura et al., 1998). Based on the morphology of the endothelial and basement membrane, capillary endothelium can be divided into continuous, fenestrated, and discontinuous endothelium (Simionescu, 1983). The continuous endothelium, which is found in all types of muscular tissues and lung, skin, and subcutaneous tissues, is the tightest and prevents the passage of materials greater than approximately 2 nm. The brain endothelium offers an even stronger barrier; only small hydrophobic molecules can cross the blood-brain barrier. Fenestrated endothelia, which occur in the intestinal mucosa, the kidney, and the endocrine and exocrine glands, contain openings of approximately 40-60 nm in diameter. However, the continuous basement membrane surrounding these capillaries prevents the passage of macromolecules larger than approximately 11 nm. Discontinuous capillaries or sinusoidal capillaries are found in the liver, spleen, and bone marrow. These capillaries have endothelial junctions of approximately 150 nm or even up to approximately 500 nm and contain either no (e.g., the liver) or a discontinuous basement membrane (e.g., the spleen and bone marrow). Leaky capillaries are also found at sites of inflammation and in tumors (Baban et al., 1998). Extravasation of particles with a diameter of up to 400 nm in certain tumors has been reported (Yuan et al., 1995). Although, other reports found no extravasation of particles larger than 100 nm in tumors (Kong et al., 2000). Thus, the size of the nanocarriers of this disclosure, like many conventional drug-carrying vesicles, may be largely dependent upon their intended use. However, it is contemplated that acoustically disrupting and modifying the permeability of target and possibly other tissues, using the methods and techniques of this specification, may allow much greater-sized vesicles to extravasate, with higher therapeutic-containing payloads, and cross other biological barriers in a far more efficient manner when compared to current drug delivery vesicles and methods known in the art.

Ocular drug and gene therapy may offer new hope for severe eye diseases such as, for example, retinitis pigmentosa and age-related macular degeneration (AMD). Many of these ocular diseases are due to a gene defect in the retina, a multilayered sensory tissue that lines the back of the eye. The blood-retinal barrier and the sclera prevent large molecules such as many conventional drug-carrying vesicles, from accessing the retina after systemic or topical applications (Duvvuri et al., 2003). However, by using the methods of the present teachings, the blood-retinal barrier and sclera may be temporarily and safely disrupted, allowing the nanocarriers of this disclosure to effectively pass said structures. In addition, intravitreal injection, which is less invasive than subretinal injection, may also be a route for ocular drug delivery using the present teachings. However, before the vesicles of this specification can reach the retina, they must travel through the vitreous, a gel-like material built up from collagen fibrils bridged by proteoglycan filaments that contain negatively charged glycosaminoglycans (Bishop, 1996). This biopolymer network may immobilize many conventional carriers with, for example, glycosaminoglycans, further binding and impeding said vesicles. Indeed, recent studies have confirmed this, where a thin layer of vitreous on top of retinal cells almost completely blocked the gene expression of cationic polyplexes and lipoplexes (Pitkanen et al., 2003). Further, cationic lipoplexes have been shown to be severely aggregated when mixed with vitreous (Peeters et al., 2005). This aggregation is most likely due to the binding of negatively charged biopolymers in the vitreous such as, for example, charged glycosaminoglycans, to the cationic lipoplexes, neutralizing their surface charge, thus leading to aggregation. These aggregated carriers may become completely immobilized in the vitreous gel, having little chance to reach retinal cells. In addition, binding of glycosaminoglycans to lipo- and polyplexes may also impede the intracellular processes which lead to successful gene expression (Ruponen et al., 2001). By following the teachings of this disclosure, optimal embodiments of the present invention may allow effective intracellular drug delivery to both the anterior and posterior portions of the eye by, for example, temporarily disrupting said structures, assisting the nanocarriers of this specification to effectively travel through the vitreous, breaking up and limiting said nanocarrier aggregation and crossing formidable membrane barriers.

Some of these extracellular barriers may be avoided, at least partially, by coating the nanocarriers of this disclosure with compounds such as, for example, polyethylene glycol (PEG), derivatives of PEG, and other polymers and materials which may prevent aggregation, reduce toxicity, prevent uptake by the mononuclear phagocytic system, enhance the circulation time in the bloodstream, and improve the journey of said nanocarriers through extracellular matrices (e.g., serum, sputum, and vitreous). The vesicles of this disclosure can be shielded with polymers, for example, by using a cationic nanocarrier with DNA covalently coupled to the shielding polymer, and subsequently mixed with the DNA. During the self-assembly of said vesicle, the DNA and the cationic carrier interact with each other, creating a slightly hydrophobic core that is surrounded by a shield of hydrophilic polymers. This method has the disadvantage that the shielding polymers can hinder the self-assembling process between the cationic carrier and the anionic DNA, especially when high amounts of shielding polymer are used. Therefore, post-shielding may also be utilized, involving the physical incorporation or covalent attachment of the shielding polymer, or other compound, to preformed nanocarriers. Further, the cationic surface of a preassembled nanocarrier also allows ionic coating by negatively charged polymers. In addition, anionic polymers such as, for example, poly (propylacrylic acid) (PPAA), may be utilized, as discussed in greater detail later in this disclosure.

The presence of hydrophilic polymers on the surface of the nanocarriers, described herein, also prevents aggregation by avoiding vesicles that can come in close proximity to each other during collision. In addition, when present in sufficient amounts, these dangling polymers protruding on the surface of said nanocarriers also avoid macromolecules that can reach, in some embodiments, the charged core of the nanocarrier. Unfortunately, they can also prevent close interactions between the nanocarriers and cell membranes. This may be overcome by reversible shielding of said vesicles with, for example, polyethylene glycol (PEG), which implies that the vesicles lose their protective shield at or in the target cells. In addition, there are normally gaps between the polymer chains that may allow small charged molecules to reach the surface of the nanocarriers. The size of these gaps depends on the degree of shielding and the chain length of the polymers. However, long PEG chains (>10 kDa) may entangle in the biopolymer network of biogels. Therefore, particles containing such long, for example, PEG chains may become immobilized in mucus or vitreous. Alternative methods for shielding include the use of synthetic polypeptides, poly(propylacrylic acid), polysaccharides, and the like, either alone or along with PEG or a derivative of PEG. Importantly, in an optimal embodiment, ends of the shielding polymers may be used to provide the nanocarriers of this disclosure with a variety of different targeting moieties.

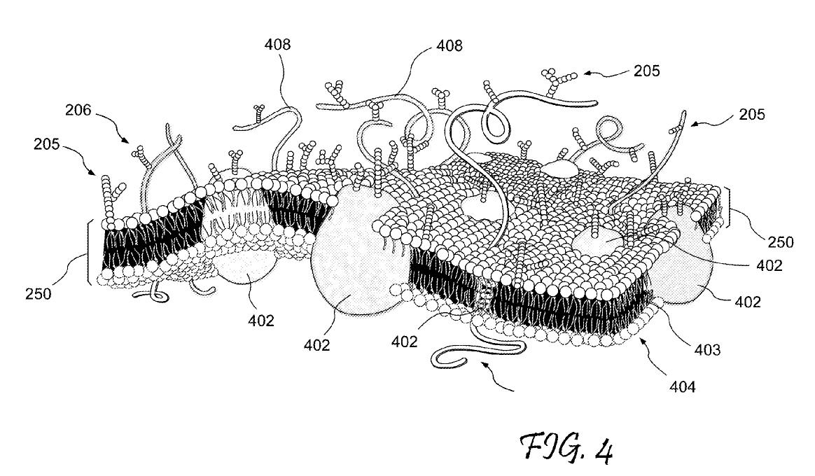

Intracellular barriers. Even though parenteral administration of pharmaceuticals ensures delivery to the systemic circulation, a drug still must traverse the semipermeable, plasma membrane bordering target cells, before reaching the interior of the cells of target tissues as well as, possibly, intracellular membranes, depending primarily on the therapeutic and its intended site of use. These membranes are biologic barriers that selectively inhibit the passage of larger drug molecules (e.g., therapeutic macromolecules) and are composed primarily of a bimolecular lipid matrix, containing mostly cholesterol and phospholipids (FIG. 4). The lipids provide stability to the membrane and, a most important characteristic for the present teachings, determine its permeability characteristics. Globular proteins of various sizes and compositions are embedded in the matrix; they are involved in transport and function as receptors for cellular regulation. An illustration of the Fluid Mosaic Model of plasma membranes developed by Singer et al. in 1972 is shown in FIG. 4. All current evidence in modern biology is compatible with the Fluid Mosaic Model, and this embodiment is broadly accepted among scientists from multiple disciplines.

According to the Fluid Mosaic (FIG. 4), a membrane is a liquid in two dimensions, but an elastic solid in the third (250). Importantly, and for the successful application of the present teachings, this elasticity is critical and contributes substantially to the self-healing properties of biological membranes. In this model, proteins float freely in the fluid bilayer (402), and are also held in place by their lipophilic sections which are attracted to the fatty middle layer of the membrane (403), accounting for their high mobility in biological membranes. According to the Fluid Mosaic Model (FIG. 4), the basic structure of the membrane is provided by the phospholipid molecules. For example, unsaturated fatty acid tails make a membrane more liquid, while the addition of cholesterol to the fatty layer makes said membrane more viscous and more repellent to water. Proteins are responsible for many of the special characteristics of different types of membranes (402) controlling the ability of cells to transport molecules, receive chemical messages, and attach to adjacent cells, as well as many other characteristics and processes (402). Because lipid molecules are small when compared to proteins, there are typically many more lipid molecules than protein molecules in biological membranes—approximately 50 lipid molecules for each protein in a membrane that is 50% protein by mass. Like membrane lipids (405), membrane proteins often have oligosaccharide chains attached to them on the portion of the molecule that faces the cell exterior (206). Thus, the surface the cell presents to the exterior is rich in carbohydrate, essentially forming a cell coat. As described later in this specification, these external structures on cell membranes may represent important targeting structures for the drug-carrying vesicles (i.e., nanocarriers) of this specification (FIG. 4).

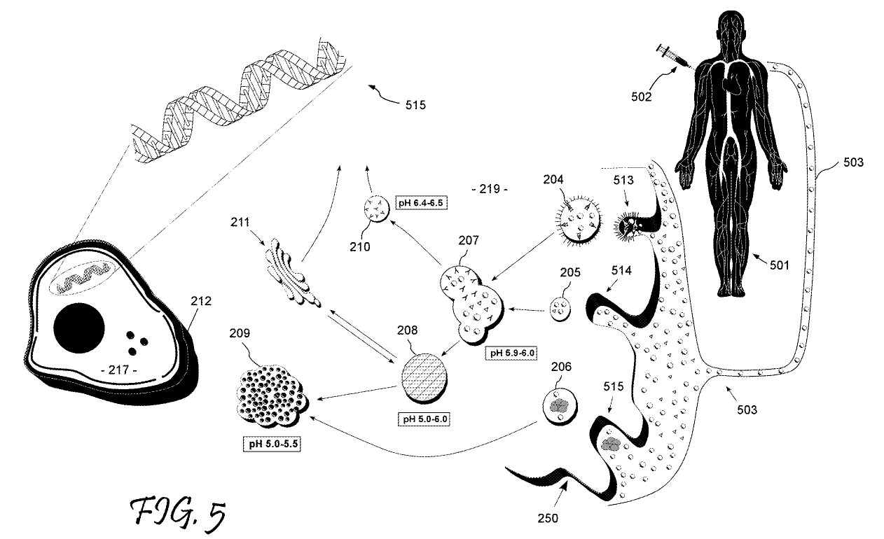

Especially troublesome intracellular barriers for the delivery of, for example, therapeutic macromolecules, are the vesicles of the endocytic pathway. FIG. 5 illustrates a schematic representation of the biological distribution of many currently used colloidal therapeutic carriers (503) following parenteral administration 502 to a patient 501. These conventional carriers are usually required to circulate in the bloodstream 503, and as reviewed previously, escape recognition by the reticuloendothelial system (RES), and avoid hepatic clearance, glomerular excretion, etc. Receptor-mediated targeting may be achieved by installing pilot/targeting moieties on the surface of these carriers, using, for example, end-functionalized block copolymers. Without wishing to be bound by any particular theory, many conventional macromolecular carriers such as, for example, nanospheres, micelles, liposomes, and the like, usually enter target cells by endocytosis (FIG. 5) where endosomes with encapsulated carriers are separated from the cell membrane by a process of inward folding (513, 514, 515). These vesicles have an increasingly acidic pH as the endosomes move toward a final destination of cellular lysosomes (209), where nearly all materials, including the endocytosed therapeutic macromolecules, will be destroyed (i.e., hydrolyzed). Thus, membranes inside the cell, including the nuclear membrane (212) may represent additional biological barriers for the delivery of therapeutic macromolecules (FIG. 5), as discussed in greater detail below. For the purposes of more clearly understanding the present teachings, endocytosis may be divided simplistically into three major processes: (1) receptor-mediated endocytosis, (2) pinocytosis, and (3) phagocytosis.

Receptor-mediated endocytosis (FIG. 5 [513]) is typically prompted by the binding of a large extracellular molecule—such as a protein—to a receptor on the cell membrane. Many conventional intracellular drug delivery vesicles utilize receptor-mediated endocytosis for entry into the cell cytoplasm. The receptor sites utilized by this process are commonly grouped together along coated pits in the membrane which are lined on their cytoplasmic surface with bristle-like coat proteins (513) (i.e., clathrin chains with the AP-2 adaptor complexes). The coat proteins are thought to play a role in enlarging the pit and forming a vesicle (204). When the receptors bind their target molecules, the pit deepens (313) until a protein-coated vesicle is released into the cytosol (204). Through receptor-mediated endocytosis, active cells are able to take in significant amounts of particular molecules (e.g., ligands), including ligand-labeled, drug-containing vesicles that bind to the receptor sites extending from the cytoplasmic membrane into the extracellular fluid surrounding the cell. However, vesicles produced via receptor-mediated endocytosis may internalize other molecules in addition to ligands, although the ligands are usually brought into the cell in higher concentrations (FIG. 5).

By the mechanisms of pinocytosis, a cell is usually able to ingest droplets of liquid from the extracellular fluid (FIG. 5). This is a constant process with the rate varying from cell to cell. For example, a macrophage internalizes approximately 25% of its volume every hour. All solutes found in the medium outside the cell (503) may become encased in the vesicles formed via this process (205). Those present in the greatest concentration in the extracellular fluid are likely to be the most concentrated in the membrane vesicles (205). Pinocytic vesicles tend to be smaller than vesicles produced by other endocytic processes (205), with the major purpose of pinocytosis being to take in a wide range of extracellular molecules and atoms including minerals. Entry of for example, many smaller therapeutic nucleic acids, by conventional methods of administration, are believed to be almost entirely by pinocytosis (Akhtar et al., 2007). Also, cholesterol-containing particles called LDLs, composed of cholesterol and proteins, are taken up by pinocytosis. The other purpose of pinocytosis is also important, but less obvious. In order for cells to communicate with each other, they must have the ability to constantly secrete hormones, growth factors, neurotransmitters for nervous system function, etc. As mentioned previously, secretion of membrane is also critical for repairing cell membrane damage. While some of this membrane is synthesized and stored inside the cell, much comes from the cell surface that is continually internalized by pinocytosis (514 and 205). Therefore, this constant process means there is a considerable amount of membrane internalized to quickly recycle to the surface for secretion, and most importantly for the present teachings, a ready source of renewable materials for membrane repair.

Phagocytosis is the process by which cells ingest large objects (FIG. 5 [515]) such as prey cells or chunks of dead organic matter, and is probably the most well-known manner in which a cell imports materials from the extracellular fluid. This debris is then sealed off into larger vacuoles (206). Lysosomes (209) then merge with this vacuole, turning it into a digestive chamber. The products of the digestion are then released into the cytosol. Macrophages are cells of the immune system that specialize in the destruction of antigens (e.g., bacteria, viruses, and other foreign particles) by phagocytosis. With all three endocytic mechanisms, the vessels formed can be broadly termed endosomes (FIG. 5).

Typically, once endosomal vessels have formed and gained entrance to the cell cytoplasm, some of the ingested molecules are selectively retrieved and recycled to the plasma membrane (210), while others pass on into late endosomes (FIG. 5 [208]). This is the first place that endocytosed molecules usually encounter caustic enzymes (i.e., primary hydrolases). The interior of the late endosomes is mildly acidic, allowing for the beginning of hydrolytic digestion. Once freed into the cytoplasm, several small vesicles produced via endocytosis may come together to form a single entity (207). This endosome generally functions in one of two ways. Most commonly, endosomes transport their contents in a series of steps to a lysosome (209), which subsequently digests the materials. In other instances, however, endosomes are used by the cell to transport various substances between different portions of the external cell membrane. An endosome that is destined to transfer its contents to a lysosome generally goes through several transformations along the way. In its initial form, when the structure is often referred to as an early endosome, the specialized vesicle contains a single compartment. Over time, however, chemical changes in the vesicle take place and the membrane surrounding the endosome folds in upon itself in a way that is similar to the invagination of the plasma membrane. In this case, however, the membrane is not pinched off. Consequently, a structure with multiple compartments, termed a multivesicular endosome, is formed (207). The multivesicular endosome (207) is an intermediate structure in which further chemical changes, including a significant drop in pH, take place as the vesicle develops into a late endosome (308). Though late endosomes (308) are capable of breaking down many proteins and fats, a lysosome (309) is needed to fully digest all of the materials contained within multivesicular and late endosomes. Therefore, the necessity of escape of endocytosed therapeutic macromolecules from these endosomal and other vesicles is a key problem in intracellular drug delivery. The present teachings represent broadly applicable methodologies to solve these and other critical challenges.

Cytosolic sequestration and degradation is yet another problem especially for, for example, nucleic acid macromolecules (FIG. 5 [515]) that require entry into the cell nucleus for therapeutic efficacy. The cytoplasm (FIG. 3A [219]) is composed of a network of microfilamental and microtubule systems, and a variety of subcellular organelles bathing in the cytosol. The cytoskeleton (218) is responsible for the mechanical resistance of the cell, as well as the cytoplasmic transport of organelles and large complexes. The mesh-like structure of the cytoskeleton (218), the presence of organelles, and the high protein concentration impose an intensive molecular crowding of the cytoplasm which limits the diffusion of large-sized macromolecules (Luby-Phelps, 2000). Indeed, the cytoplasm (204) probably constitutes a significant diffusional barrier to gene transfer to the nucleus. Embodiments of the present invention may be of assistance in increasing cytoplasmic diffusion of therapeutic and molecules and macromolecules inside target cells and tissues by methods including those described previously.

Sequence-specific gene silencing, using small interfering RNA (siRNA), is now being evaluated in clinical trials, and is a Nobel prize-winning technology with considerable therapeutic potential. However, efficient intracellular siRNA delivery to specific target sites in the body following systemic administration is the most important hurdle for widespread use of RNAi in the clinic (Akhtar et al., 2007). At present, it is widely thought that cellular uptake of siRNA occurs via pinocytosis, most likely in a manner similar to that observed for other gene-silencing molecules such as oligonucleotides and ribozymes (Akhtar et al., 2007). Thus, preferred embodiments of the present invention should offer ideal methods for successful siRNA delivery. In order for these applications to be successful clinically, the RNA duplex structure may be modified chemically such as, for example, modifications to the backbone, base, or sugar of the RNA. In addition, transfection conditions will need to be optimized for each particular application, including, for example, the duplex siRNA (e.g., chemistry, length, and charge), the nature of the target gene/gene product. In particular, suspending the siRNA in some type of gel or polymer matrix such as, for example, a hyaluronic acid gel or cationic polymer such as polyethyleneimine, previous to being encapsulated in a nanocarrier, with said suspended therapeutic, then delivered by methods of the present teachings. Following diffusion of the suspended siRNA through the target cell membrane, the suspending medium then slowly dissolves, allowing “timed-released” siRNA delivery and other nucleic acids (e.g., oligonucleotides) directly into the target cell cytoplasm over extended periods.

The nucleus (FIG. 2A [217]) is surrounded by a double membrane (212) which compartmentalizes nuclear and cytoplasmic reactions. Besides its key role in regulating nucleocytoplasmic transport, the nuclear membrane provides a structural support for the attachment of other macromolecular structures such as the nuclear lamina, nucleoskeleton, cytoskeleton, and chromatin. The nuclear envelope is the ultimate obstacle to the nuclear entry of, for example, therapeutic plasmid DNA, where the inefficient nuclear uptake of said plasmid from the cytoplasm was recognized decades ago (Capecci, 1980). Indeed, no more than 0.1-0.001% of cytosollically injected plasmid DNA could be successfully transcribed (Capecci, 1980). However, nucleocytoplamic transport of macromolecules through the nuclear membrane is a fundamental process for the metabolism of eucaryotic cells and involves nuclear pore complexes (NPCs) that form an aqueous channel through the nuclear envelop (not illustrated; Laskey, 1998). While molecules smaller than ˜40 kilodalton (kDa) can diffuse through the NPC passively, plasmids and other macromolecules larger than ˜60 kDa usually comprise a specific targeting signal, the nuclear location sequence (NLS), to transverse the NPC successfully in an energy-dependent manner (Talcott et al., 1999). Now it is widely accepted that the size of expression cassettes constitutes a major impediment to nuclear targeting. DNA fragments diffuse passively into the nucleus if their size is small enough (i.e., a 20 base pair double-stranded oligomer is approximately equivalent in size to a 13 kDa polypeptide), where oligonucleotides efficiently escape the transport barrier of the cytoplasm and nuclear envelope (Lechardeur et al., 2002). While larger, for example, plasmid DNA and DNA fragments require nuclear localization sequences or other methods that facilitate active transport to the nucleus and through its membrane. Embodiments of this disclosure may be of assistance in nuclear entry of a variety of compounds by cavitation-mediated kinetic energy, as well as other mechanisms.

The aforementioned examples are only representative of the many potential embodiments of this disclosure. Most importantly, these embodiments are intended to be exemplary only, and therefore non-limiting to the present specification. A plethora of variables can be altered with each of these examples, as well as many other embodiments; therefore, a wide variety of applications, techniques, materials, and other properties are available for the preparation of targeted and non-targeted nanocarriers for acoustically mediated drug delivery, as well as the administration and activation procedures of said components. Optimally, said therapeutics and/or materials will be designed and engineered for specific levels of acoustic sensitivity.

### Nanocarriers Comprised of Biodegradable Triblock Polymers

A new approach in the development of polymeric biomaterials, including the novel materials for drug and gene delivery, is to custom design the polymer to tailor its properties for the desired application. Since poly(amino acid)s are structurally related to natural proteins, and as reviewed herein, the synthesis of amino-acid-based polymers has been explored in detail as a potential source of new biomaterials. Starting from about 1970, the use of both homo- and copolymers of amino acids was studied for a variety of biomedical applications (Hench et al., 1982; Lyman, 1982; Anderson et al., 1985; and Lescure et al., 1989). The early studies revealed that most poly(amino acid)s could not be considered as potential biomaterials due to their immunogenicity and unfavorable mechanical properties. So far, only a small number of poly(γ-substituted glutamates) and copolymers thereof (Sidman et al., 1980; Sidman et al., 1983; Anderson et al., 1985; and Bhaskar et al., 1985) have been identified as promising candidate materials for biomedical applications.

To improve the unfavorable physicomechanical properties of most poly(amino acid)s, amino acids have been used as monomeric building blocks in polymers that do not have the conventional backbone structure found in peptides. Collectively, these materials are referred to as “non-peptide amino-acid-based polymers” or as “amino-acid-derived polymers with modified backbones.” These materials are typically classified into four major groups:
1. Synthetic polymers with amino acid side chains—these polymers consist of amino acids or peptides which have been grafted as side chains onto a synthetic polymer backbone.
2. Copolymers of amino acids and non-amino acid monomers—copolymerization of α-L-amino acids with non-amino acid monomers has been achieved through a variety of reaction schemes, leading to polymers with a wide range of structures.
3. Block copolymers contain peptide or poly(amino acid) blocks—these polymers mostly consist of A-B or A-B-A block copolymers, where A is poly(ethylene glycol) and B is a conventional poly(amino acid) or peptide. Such systems represent promising materials for the delivery of therapeutic agents by control of supramolecular assemblies. Peptosomes, as reviewed herein, are one such application.
4. Pseudo-poly(amino acid)s—naturally occurring amino acids are linked by non-amide bonds, such as ester, iminocarbonate, and carbonate bonds. The resulting polymers contain the same monomeric building blocks as conventional poly(amino acid)s, but do not have a peptide-like backbone structure. A number of tyrosine-derived pseudo-poly(amino acid)s have been extensively studied and found quite suitable for biomedical applications (Bourke et al., 2003).

The natural amino acid L-tyrosine is a major nutrient having a phenolic hydroxyl group. This feature makes it possible to use derivatives of tyrosine dipeptide as a motif to generate diphenolic monomers, which are important building blocks for the design of biodegradable polymers. Particularly useful monomers are desaminotyrosyl-tyrosine alkyl esters (abbreviated as DTR, where R stands for the specific alkyl ester used). Using this approach, a wide variety of polymers are known in the art. The present invention utilizes several of these tyrosine-derived polycarbonates, polyarylates, and polyethers, complexed with, for example, polyethylene glycol (PEG), and then structurally optimized for use in acoustically-mediated, intracellular drug delivery in vivo.

Several seminal research publications and patents, biodegradable polymers based on desaminotyrosyl-tyrosine alkyl esters, their discovery, synthesis, characterization and possible utility, are known in the art. However, none of these publications include any information or even mention the possibility of using desaminotyrosyl-tyrosine alkyl esters as delivery vesicles in acoustically mediated drug delivery in vivo, as described by the teachings of the present invention.

Many important peer-reviewed research papers supplying important background material (e.g., materials; synthesis methods, procedures, and techniques) for the present teachings, and include those by Kohn et al., (1986a); Kohn et al., (1986b); Kohn et al., (1986c); Kohn, (1990; Pulapura et al., (1990); Engelberg et al., (1991); Pulapura et al., (1992a); Pulapura et al., (1992b); Silver et al., (1992); Nathan et al., (1993); Ertel et al, (1994); Fiordeliso et al., (1994); Kohn et al., (1994); Parkinson et al., (1994); Ertel et al., (1995); Choueka et al., (1996); Kohn, (1996); Tangpasuthadol et al., (1996); Greco et al., (1997); Poiani et al., (1997); Brocchini et al., (1998); Hooper et al., (1998); Suarez et al., (1998); James et al., (1999); Yu et al., (1999a); Yu et al., (1999b); Belu et al., (2000); Hooper et al., (2000); Tangpasuthadol et al., (2000a); Tangpasuthadol et al., (2000b); Chaikof et al., (2002a); Chaikof et al., (2002b); Parang et al., (2002); Raspe et al., (2002); Schachter et al., (2002); Taylor et al., (2002); Auguste et al., (2003); Bourke et al., (2003a); Bourke et al., (2003b); Bourke et al., (2004); Kohn, (2004a); Kohn et al., (2004b); Nardin et al., (2004); Sharma et al., (2004); Smith et al., (2004); Weber et al., (2004); Abramson et al., (2005); Kohn et al., (2005a); Kohn et al., (2005b); Sheihet et al., (2005); Weber et al., (2005); Auguste et al., (2006); Bae et al., (2006); Bailey et al., (2006); Meechaisue et al., (2006); Sousa et al., (2006); Liu et al., (2007); Sheihet et al., (2007); and Weber et al., (2007).

U.S. patent Nos. important to the teachings of the present invention include U.S. Pat. Nos. 4,638,045; 4,806,621; 4,863,735; 4,980,449; 5,099,060; 5,140,094; 5,194,570; 5,198,507; 5,216,115; 5,219,564; 5,242,997; 5,264,537; 5,317,077; 5,455,027; 5,587,507; 5,658,995; 5,670,602; 5,670,602; 5,877,224; 6,048,521; 6,103,255; 6,120,491; 6,284,862; 6,319,492; 6,337,198; 6,359,102; 6,475,477; 6,517,824; 6,602,497; 6,852,308; 7,005,454; and 7,250,154. U.S. patent applications relevant to the present teachings include Ser. No. 10/514,215, filed on May 15, 2003; Ser. No. 10/952,274, filed on Sep. 27, 2004; Ser. No. 11/001,250, filed on Dec. 1, 2004; Ser. No. 11/191,181, filed on Jul. 27, 2005; and Ser. No. 10/952,202, filed on Feb. 16, 2006.

While stabilized triblock copolymers have been used in a form of acoustically mediated drug delivery (see, e.g., U.S. Pat. No. 6,649,702), none of the biodegradable compositions described herein have been used in such an application. The disclosure of all publications, patents, and patent applications listed in paragraphs [0149] and [0150] are hereby incorporated by reference herein in their entirety for all purposes.

### Design of Tyrosine-Derived Diphenolic Monomers

Diphenols, such as Bisphenol A, are frequently used in industry because their aromatic backbone structures can significantly increase the stiffness and mechanical strength of polymers (Bourke et al., 2003). However, Bisphenol A and other industrially used diphenols are cytotoxic and therefore cannot be used as monomers in degradable biomaterials. Thus, there was a significant need for a non-cytotoxic, diphenolic monomer that can be used as a building block in the design of degradable implant materials. This need was addressed by the development of several tyrosine-based monomers. Tyrosine is the only major, natural nutrient containing an aromatic hydroxyl group. In view of the non-processability of conventional poly(L-tyrosine), the development of a tyrosine-based pseudo-poly(amino acid) was a clear alternative. In this context, derivatives of tyrosine dipeptide can be regarded as diphenols and may be employed as replacements for the industrially used diphenols in the design of medical implant and other materials. This approach led, for the first time, to tyrosine-derived polymers with favorable engineering properties (Bourke et al., 2003).

The protecting groups used to block the N- and C-termini of a tyrosine dipeptide have a significant impact on the properties of the resulting polymers (Bourke et al., 2003). The challenge of early studies was to identify suitable protecting groups that will lead to non-toxic, fully degradable polymers with good engineering properties. The combination of these different properties within one single design proved to be a difficult task, and early investigations did not lead to readily processable materials (Kohn et al., 1987; and Kohn et al., 1988). Later, it was recognized that the number of inter-chain hydrogen bonding sites per monomer unit had to be minimized (Pulapura et al., 1990). These studies led to the replacement of one tyrosine molecule with desaminotyrosine[3-(4′-hydroxyphenyl)propionic acid]. The resulting desaminotyrosyl-tyrosine alkyl esters were identified as fully biocompatible replacements for Bisphenol A and other industrial diphenols in a wide range of polymers (Kohn, 1991a; Kohn, 1991b; and Kohn, 1991c).

### Tyrosine-Derived Triblock Polymers

The triblock copolymers of the present invention are derived from water-soluble, hydrophilic, and non-toxic polymer end blocks, and a hydrophobic polyarylate oligomer middle block of a biocompatible, non-toxic aliphatic or aromatic diacid; and a derivative of a tyrosine-derived diphenol. Thus, according to one aspect of the present invention, polyarylate triblock copolymers are provided having an A-B-A structure, wherein each A end block is a water-soluble, hydrophilic, and non-toxic polymer end block; and the B middle block is an polyarylate oligomer with the same as or different repeating units having the structure of Scheme 1, wherein Z is between 2 and to about 100, R₁ is CH═CH or (CH₂)ₙ wherein n is from 0 to 18, inclusive; R₂ is selected from hydrogen and straight and branched alkyl and alkylaryl groups containing up to 18 carbon atoms; and R is selected from a bond or straight and branched alkyl and alkylaryl groups containing up to 18 carbon atoms.

The endblocks are preferably poly(alkylene oxides) having the structure: R₃—[(CH₂—)ₐCH₃—O—]ₘ—. Wherein m for each A end block is independently selected to provide a molecular weight for each A between about 1,000 and about 15,000 and R₃ for each A, and within each A, is independently selected from hydrogen and lower alkyl groups containing from one to four carbon atoms; and a is an integer greater than or equal to one. In a preferred embodiment, the end blocks have the structure CH₃O—[CH₂CH₂O—]ₘ.

The preferred embodiments of the present invention provide nanocarrier-encapsulated biologically or pharmaceutically active compounds, wherein the therapeutic containing nanocarriers are present in an amount sufficient for effective, acoustically-mediated, site-specific delivery. The carrier may be an aqueous solution or a polymeric drug delivery matrix. In a most preferred embodiment, nanocarriers that are either targeted or untargeted, are utilized for site-specific, intracellular delivery of said active compounds, by administering to the patient an effective amount of said compounds encapsulated by the polymer nanocarriers of the present disclosure, and acoustically disrupting said nanocarrier.

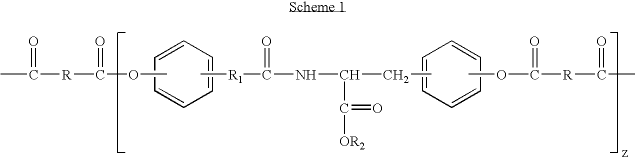

The triblock copolymers degrade by hydrolysis into the original starting materials, i.e., the tyrosine-derived diphenols, the dicarboxylic acids, and the water-soluble, hydrophilic, and non-toxic oligomer end blocks. The inventive copolymers are highly hydrophilic, which is advantageous for the nanocarrier drug delivery systems of the present invention. However, the hydrophilic:hydrophobic balance of the copolymers, which is the characteristic likely to determine their acoustic sensitivity, can be varied in several ways. The ester of the pendant chain of the diphenol can be changed, with longer-chain ester groups resulting in increased hydrophobicity. Increasing the molecular weight of the A end blocks, for example, by increasing the number of carbons in the alkylene group of a poly(alkylene oxide) will also increase hydrophobicity. Changing the dicarboxylic acid will also change the hydrophilic:hydrophobic balance.

While certain of the above-mentioned polymer properties are individually well-known in the prior art, the combination of properties within a single composition, especially when used in acoustically mediated drug delivery, is new and represents a significant technological advance that has broad utility in the fields of drug and gene delivery. Specifically, the family of triblock copolymers described herein has at least three major, distinguishing advantages over other triblock copolymers:
1. The family of triblock copolymers is fully resorbable after being introduced into the patient. As the compositions are derived exclusively of non-toxic building blocks, the triblock copolymers themselves as well as the expected degradation products in vivo, are non-cytotoxic, and biocompatible.
2. The family of triblock copolymers self-assemble to form hollow nanocarriers with the above-mentioned low critical micelle concentration (CMC) and remain stable even under very high dilution, and are acoustically sensitive depending on the composition of said triblock copolymers.
3. The family of triblock copolymer provides a wide range of structural parameters which can be changed by those skilled in organic synthesis to derive triblock copolymers that are closely related to each other in chemical structure, while allowing the tailoring of key properties (such as the rate of bioresorption, the physical characteristics of the nanocarriers formed, and the release profiles obtained for the encapsulated therapeutic). Most importantly for use with the present invention, these structural parameters can be optimized yielding polymers that form aggregates with specific sensitivity to disruption by acoustic energy for both in vitro and in vivo applications.

### Preferred-Biodegradable Triblock Polymer Compositions

The following are examples of preferred chemical embodiments of the present invention, and should not be viewed as limiting. In many preferred embodiments, the components and structure of said triblock polymer nanocarriers will be chosen and optimized so the vesicle will have a specific level of acoustic responsiveness.

The copolymers of the present invention are A-B-A type triblocks, where the A end blocks are water-soluble, hydrophilic, and non-toxic, preferably selected from poly(alkylene oxides); and the hydrophobic middle B block is either a polyarylate or polycarbonate. In a preferred polyarylate embodiment, the mid-block is copolymerized from a tyrosine-derived diphenol and a diacid, linked together by an ester bond between the phenolic hydroxyl group of the tyrosine-derived diphenol, and the carboxylic acid group of the diacid. In another preferred embodiment, the polycarbonate mid-block is copolymerized from the same dihydroxy monomers.

Among the more preferred poly(alkylene oxides) end blocks are polyethylene glycol, polypropylene glycol, polybutylene glycol, Pluronic® polymers, and the like. Polyethylene glycols are especially preferred.

The polyarylate middle blocks of the present invention are prepared by condensation of a diacid with a diphenol according to the method described by U.S. Pat. No. 5,216,115, in which diphenol compounds are reacted with aliphatic or aromatic dicarboxylic acids in a carbodiimide mediated direct polyesterification using 4-(dimethyl-amino)-pyridinium-p-toluene sulfonate (DPTS) as a catalyst. The disclosures of U.S. Pat. No. 5,216,115, in this regard are incorporated herein by reference. Bis-diacids are selected as the polyarylate middle blocks permitting the A end blocks to be coupled at each end of the copolymer.

The diphenol compounds are tyrosine-derived diphenol monomers of U.S. Pat. Nos. 5,587,507 and 5,670,602; both of which are incorporated herein by reference. The polyarylates are prepared using tyrosine-derived diphenol monomers having the structure of Scheme 7, wherein R₁ and R₂ are the same as described above with respect to Scheme 1. The preferred diphenol monomers are desaminotyrosyl-tyrosine carboxylic acids and esters thereof, wherein R₁ is —CH₂—CH₂—, which are referred to as DT esters. For purposes of the present invention, the ethyl ester (R₂=ethyl) is referred to as DTE, the benzyl ester (R₂=benzyl) as DTBn, and so forth. For purposes of the present invention, the desaminotyrosyl-tyrosine free carboxylic acid=(R₂=hydrogen) is referred to as DT.

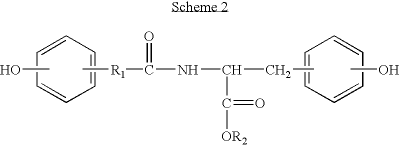

The polyarylate dicarboxylic acids have the structure: HOOC—R—COOH (Scheme 3) wherein R is the same as described above with respect to Scheme 2, and preferably contains up to 12 carbon atoms. R is preferably selected so that the dicarboxylic acids employed as starting materials are either important naturally-occurring metabolites, or highly biocompatible compounds. Preferred Scheme 3 dicarboxylic acids therefore include the intermediate dicarboxylic acids of the cellular respiration pathway known as the Krebs cycle. These dicarboxylic acids include alpha-ketoglutaric acid, succinic acid, fumaric acid, malic acid, and oxalacetic acid, for which R is —CH₂—CH₂—C(═O)—, —CH₂—CH₂—, —CH═CH═, —CH₂—CH(—OH)— and —CH₂—C(═O)—, respectively.

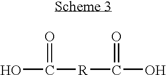

Another naturally-occurring, preferred dicarboxylic acid is adipic acid (R═[—CH₂—]₄), found in beet juice. Other preferred biocompatible dicarboxylic acids include oxalic acid (no R), malonic acid (R ═[—CH₂—]), glutaric acid (R═[(—CH₂—]₃) pimelic acid (R═[(—CH₂—]₅), suberic acid (R═[(—CH₂—]₆), and azalaic acid (R═[(—CH₂—]₇). In other words, among the dicarboxylic acids suitable for use in the present invention are compounds in which R represents (—CH₂—)_{z}, wherein z is an integer between 0 and 12, inclusive. A preferred class of highly biocompatible aromatic dicarboxylic acids are the bis(p-carboxyphenoxy) alkanes such as bis(p-carboxyphenoxy) propane.

The triblock copolymers of the present invention may be iodine- and bromine-substituted, which renders the copolymers radio-opaque. These copolymers and their methods of preparation are disclosed by U.S. Pat. No. 6,475,577; the disclosures of which are incorporated herein by reference in their entirety. Radio-opaque copolymers include repeating structural units in which one or more hydrogens of an aromatic ring, an alkylene carbon, or both, are replaced with an iodine or bromine atom. The triblock copolymers of the present invention may be similarly iodine- and bromine-substituted. Copolymers according to the present invention comprising the repeating structural units of Scheme 2 are radio-opaque when copolymerized with radio-opaque monomers so that the copolymers also contain radio-opaque repeating structural units, preferably one or more of the A or B blocks in which one or more hydrogens of an aromatic ring, an alkylene carbon, or both, have been replaced with an iodine or bromine atom.

The molecular weights of the triblock copolymers can be controlled either by limiting the reaction time or the ratios of the components. Molecular weights can also be controlled by the quantity of the carbodiimide coupling reagent employed.

Preferred polyarylate oligomers have weight average molecular weights between approximately 1,000 and 50,000 daltons, preferably between approximately 3,000 and 25,000 daltons, and more preferably between approximately 5,000 and 15,000 daltons. Molecular weights are calculated by gel permeation chromatography relative to polystyrene standards in tetrahydrofuran without further correction. The triblock copolymers thus have weight average molecular weights between approximately 2,500 and 75,000 daltons, preferably between approximately 5,000 and 50,000 daltons, and more preferably between approximately 10,000 and 25,000 daltons.

Preferred polycarbonates for use with the present invention have weight-average molecular weights ranging between approximately 1,000 and 100,000 daltons, preferably between approximately 3,000 and 50,000 daltons, and more preferably between approximately 10,000 and 25,000 daltons. The triblock copolymers thus have weight average molecular weights between approximately 2,500 and 130,000 daltons, preferably between approximately 5,000 and 80,000 daltons, and more preferably between approximately 10,000 and 50,000 daltons.

The triblock copolymers are prepared by the reaction of a non-functionalized poly(alkylene oxide) mono-alkyl ether with an excess of either the dicarboxylic acid (mediated by a coupling agent such as dicyclohexyl carbodiimide). The following is a specific example of this general design (Scheme 4), illustrating the synthesis of PEG-oligo-(DTO suberate)-PEG: the molecular weights of the triblock copolymers can be controlled either by limiting the reaction time or the ratios of the components. Molecular weights can also be controlled by the quantity of the carbodiimide coupling reagent employed.

As described herein, the triblock copolymers degrade by hydrolysis into the original starting materials, i.e., the tyrosine-derived diphenols, the dicarboxylic acids, and the water-soluble, hydrophilic, and non-toxic polymer end blocks. The copolymers are highly hydrophilic, which is advantageous for nanocarrier drug delivery systems described herein. However, the hydrophilic:hydrophobic balance of the copolymers can be varied in several ways. The ester of the pendant chain of the diphenol can be changed, with longer-chain ester groups increasing the hydrophobicity. Increasing the molecular weight of the A end blocks, for example, by increasing the number of carbons in the alkylene group of a poly(alkylene oxide), will also increase hydrophobicity. Changing the dicarboxylic acid will also change the hydrophilic:hydrophobic balance.

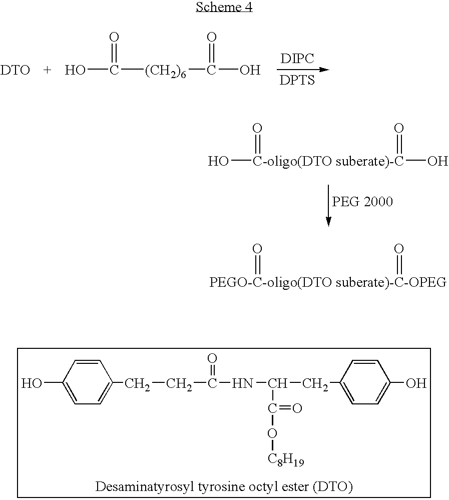

### Nanocarrier Stabilization

The polymersomes of embodiments of this disclosure may possess functional groups which may be crosslinked or stabilized by a variety of processes, methodologies, and procedures. Cross-linking said polymers is particularly useful in applications requiring additional stability of the nanocarrier corona, altered acoustic responsiveness, and/or increased retention capabilities of the encapsulated materials, such as, for example, nucleic acids. Crosslinked copolymers covalently interconnected include (1) completely crosslinked nanocarriers, having all corona components covalently interconnected into a giant single molecule; (2) cross-linked nanocarriers having interconnected components throughout the entire surface of said nanocarrier; and (3) partly cross-linked nanocarriers containing patches of interconnected components. Possible stabilization techniques include cross-linking by sulfur to form disulfide linkages, cross-linking using organic peroxides, cross-linking of unsaturated materials by means of high-energy radiation, photopolymerization, cross-linking with dimethylol carbamate, and the like.

One of the most preferred stabilization techniques for use with the present invention is photopolymerization, which is a methodology that uses light to initiate and propagate a polymerization reaction to form a linear, or cross-linked polymeric structure. This technology has been widely explored in a variety of industries for several applications including, for example, the coating industry, the paint and printing ink industries, in adhesives, and in composite materials. Recently, the use of photopolymerization has been proposed for the production of biomaterial-based polymer networks that could be differentially fabricated for a variety of applications including embodiments of this specification.

Photopolymers have broad utility in drug delivery because of a combination of properties held by photopolymerizable precursors and/or photopolymerized polymer networks. Exemplary characteristics include (1) ease of production, (2) possibility of carrying out photopolymerization in vivo or ex vivo, (3) spatial and temporal control of the polymerization process, (4) versatility of formulation and application, and (5) the possibility of entrapping a wide range of substances. Further, especially important characteristics for use with the present teachings include the ability to store photopolymerization formulation ingredients in easily accessible conditions until use such as, for example, in a clinical situation when said components need be immediately mixed together before production of polymer networks.

A wide variety of sources are known to those skilled in the art, describing techniques, procedures, and methods for photopolymerization and biomedical applications, such as, for example, drug delivery using the nanocarriers of this disclosure and other structures. Preferred publications include, but are not limited to: Photoinitiation, Photopolymerization, and Photocuring: Fundamentals and Applications (Fouassier, 1995); Microparticulate Systems for the Delivery of Proteins and Vaccines (Drugs and the Pharmaceutical Sciences: a Series of Textbooks and Monographs) (Cohen et al., 1996); Polyurethanes in Biomedical Applications (Lamba et al., 1998); Chemical and Physical Networks: Formation and Control of Properties (The Wiley Polymer Networks Group Review) (Nijenhuis et al., 1998); Polymeric Drugs & Drug Delivery Systems (Ottenbrite et al., 2001); Biomaterials for Delivery and Targeting of Proteins and Nucleic Acids (Mahato, 2005); Drug Delivery: Principles and Applications (Wiley Series in Drug Discovery and Development) (Wang et al., 2005); Photostability of Drugs and Drug Formulations (Tønnesen, 2004); and Polymers in Drug Delivery (Uchegbu et al., 2006), the disclosures of each of which are hereby incorporated by reference herein in their entirety for all purposes.

In their simplest form, a photopolymerizable system is composed of (1) a light source, (2) a photoinitiator, and (3) a monomer. In addition, this formulation can be supplemented with other molecules (e.g., monomers, crosslinkers, excipients, bioactive molecules, drugs) to fulfill specific drug delivery applications.

The light sources that heretofore have been utilized in producing biomedical polymer networks and devices, and may be used in embodiments of this disclosure include: (1) UV lamps, (2) halogen lamps, (3) plasma arc lamps, (4) light emitting diode (LED) lamps, (5) titanium-sapphire lasers—also called fernto second pulsed lasers—and (5) other laser lamps. These sources generate a beam of light that differs in terms of emission wavelength, intensity, and associated heat.

Photoinitiators are molecules responsible for initiating the polymerization reaction by producing reactive species upon light absorption. There are many different photoinitiator molecules know in the art, some of which are suitable for use in the present teachings. A partial list includes eosin Y, 1-cyclohexyl phenyl ketone; 2,2-dimethoxy-2-phenylacetophenone (DMPA), 2-hydroxy-1-[4-hydroxyethoxy)phenyl]-2-methyl-1-propanone, Irgacure 651, camphorquinone/amine, where the amine is triethylamine, triethanolamine, or ethyl 4-N—N-dimethylamino benzoate; and the like. A photoinitiator can induce a polymerization reaction directly or in the presence of other molecules. A system composed of a photoinitiator and other molecule(s) may have a synergistic function, and said mixtures are normally referred to as a Photoinitiator System (PIS). When a photopolymerizable formulation is irradiated with an appropriate light source, a series of events takes place. In the presence of a PIS, the photosensitizer will absorb said light energy, thus passing into an excited state. Successively, the photosensitizer has either to transfer said energy to the photoinitiator or to react with the photoinitiator itself. Both situations will cause the photoinitiator to produce reactive species (e.g., cationic, anionic, or free radicals). In the absence of a photosensitizer, the excited photoinitiator forms reactive species directly. Further, formulations may contain other molecules, called accelerators, which speed up these initial steps. Once the reactive species are generated, polymerization is initiated by (1) photocleavage, (2) hydrogen abstraction, or (3) generation of cationic species. The first two mechanisms of initiation will generate a free radical photo-induced polymerization, which is probably the most commonly used methodologies with biomaterials.

Commercially and non-commercially available molecules and macromolecules used as photopolymerizable monomers and macro-monomers (i.e., macromers) have one primary feature in common: Their backbone needs to have a photopolymerizable residue that normally is located at one or at both ends of the molecule. Photopolymerizable monomers already known in the art and suitable for use with the present teachings include (1) (di)methacrylic or (di)acrylic derivatives of PEG and its derivatives; (2) poly(ethylene oxide) poly(vinyl) alcohol (PVA) and its derivatives; (3) PEG-polystyrene copolymers (PEG)-(PST); (4) ethylene glycol-lactic acid copolymers (i.e., nEGmLA, where n and m are the number of repeat units of EG and LA, respectively); (5) ethylene glycol-lactic acid-caprolactone copolymers (nEGmLA CL); (6) PLA-b-PEG-b-PLA; (7) PLA-g-PVA; (8) poly(D,L-lactide-co-ε-caprolactone); (9) (poly)-anhydrides; (10) 27 anhydrides; (11) urethanes; (12) polysaccharides; (13) dextran; (14) collagen; (15) hyaluronic acid; (16) diethyl fumarate/poly(propylene fumarate); and (17) the like.

The introduction of specific properties (e.g., cell and protein adhesiveness or non-adhesiveness, mechanical strength and acoustic sensitivity, degradation rate, absence or limited mass transport constraints) in polymerized networks can be achieved by selecting appropriate monomers(s) and/or macromers(s), and supplemental molecules during the design of the monomer or its formulation. As an example, approaches to modify the degradation rate and cell adhesiveness of the polymerized networks are reported because of their significance for use in the present specification.

Degradation rate may be controlled by (1) the number of degradable chemical bonds in each monomer; (2) the type of degradable chemical bonds (e.g., ester, anhydride, amide); (3) the molecular weight of the monomer; and (4) the hydrophobic or hydrophilic nature of the monomer. While the number and nature of degradable chemical bonds are obvious key factors, the molecular weight determines whether polymer networks will be loosely (i.e., high MW) or tightly (i.e., low MW) crosslinked. In the second case, the degradation rate is slow because degradable linkages are hindered within the densely cross-linked network. In addition, hydrophobicity of highly crosslinked networks will further decrease their degradation. Obviously, degradation rate may be critical in applications involving the present teachings because it can influence, for example, the release and retention of entrapped therapeutics and other molecules.

### Nanocarrier Size

The size of the nanocarriers of embodiments of the present invention will depend on their intended use. Sizing also serves to modulate resultant biodistribution and clearance. The size of the nanocarrier can be adjusted, if desired, by the preferred method of filtering; although, other procedures known to those skilled in the art can also be used, such as shaking, microemulsification, vortexing, repeated freezing and thawing cycles, extrusion, and extrusion under pressure through pores of a defined size, sonication, and homogenization. See, for example, U.S. Pat. Nos. 4,162,282; 4,310,505; 4,533,254; 4,728,575; 4,728,578; 4,737,323; and 4,921,706, the disclosures of each of which are hereby incorporated by reference herein in their entirety for all purposes.

After intravenous injection, particles greater than 5 μm to 7 μm in diameter are often accumulated in the lung capillaries, while particles with a diameter of less than 5 μm are generally cleared from the circulation by the cells of the reticuloendothelial system. Particles in excess of 7 μm are larger than the blood capillary diameter (i.e., approximately 6 μm) and will be mechanically filtered. In the size range 70 nm to 200 nm, the surface curvature of particles may affect the extent and/or the type of protein or opsonin absorption, which plays a critical role in complement activation. The fact that particle size may change substantially upon introduction into a protein-containing medium, such as plasma, must also be taken into consideration.

Therefore, since vesicle size influences biodistribution, different-sized vesicles may be selected for various purposes. For example, for intravascular application, the preferred size range is a mean outside diameter between approximately 20 nm and approximately 1.5 μm, with the preferable mean outside diameter being approximately 750 nm. More preferably, for intravascular application, the size of the vesicles is approximately 200 nm or less in mean outside diameter, and most preferably less than approximately 100 nm in mean outside diameter. Preferably, the vesicles are no smaller than approximately 20 nm in mean outside diameter. To provide therapeutic delivery to organs such as the liver and to allow differentiation of a tumor from normal tissue, smaller vesicles, between approximately 30 nm and approximately 100 nm in mean outside diameter, are preferred. For immobilization of a tissue such as the kidney or the lung, the vesicles are preferably less than approximately 200 nm in mean outside diameter. For intranasal, intrarectal, or topical administration, the vesicles are preferably less than approximately 100 nm in mean outside diameter. Large vesicles, between 1 μm and approximately 1.5 μm in size, will generally be confined to the intravascular space until they are cleared by phagocytic elements of the immune system lining the vesicles, such as the macrophages and Kupffer cells lining capillary sinusoids. For passage to the cells beyond the sinusoids, smaller vesicles, for example, less than approximately 1 μm in mean outside diameter (e.g., less than approximately 300 nm in size), may be utilized. In preferred embodiments, the vesicles are typically administered individually, although this administration may be in some type of polymer matrix.

### Nanocarrier and Contrast Agent Targeting

As described herein, embodiments of the present invention include nanocarriers and/or certain contrast agents which may comprise various targeting components (e.g., ligands) to target the vesicle and its contents to, for example, specific cells either in vitro or in vivo. A ligand or targeting ligand is a molecule that specifically binds to another molecule, which may be referred to as a target. In another preferred embodiment, the nanocarriers of this specification can be targeted by compositions that facilitate magnetic targeting (i.e., said vesicle is guided by a magnetic field). Or in yet another embodiment, both targeting ligands and magnetic compositions (i.e., compositions that facilitate magnetic targeting) are utilized for active targeting. All of the targeting ligands and magnetic compositions described herein are considered to be within the definition of a “targeting moiety,” and are thus suitable for use with embodiments of this disclosure.

The targeting ligands incorporated in the nanocarriers of this specification are preferably substances which are capable of targeting receptors and/or tissues in vivo and/or in vitro. Preferred targeting ligands include (1) compositions facilitating magnetic targeting, (2) proteins, including antibodies, antibody fragments, hormones, hormone analogues, glycoproteins and lectins, peptides, polypeptides, and amino acids; (3) sugars such as saccharides, including monosaccharides and polysaccharides; carbohydrates, vitamins, steroids, steroid analogs, hormones, and cofactors; (4) genetic material, including aptamers, nucleosides, nucleotides, nucleotide acid constructs, and polynucleotides; and (5) peptides. Preferred targeting ligands for use with embodiments described herein include, for example, cell adhesion molecules (CAMs), among which are cytokines, integrins, cadherins, immunoglobulins and selectins; optimal genetic material for targeting includes aptamers.

A wide variety of sources is known in the art that describes techniques, procedures, and methods for active targeting of drug-containing vesicles using ligands and other structures, such as, for example, the nanocarriers of embodiments of the present invention. For enablement purposes, preferred publications include, but are not limited to Using Antibodies: A Laboratory Manual (Harlow et al., 1999); Drug Targeting Organ-Specific Strategies (Molema et al., 2001); Molecular Cloning: A Laboratory Manual (Sambrook et al., 2001); Drug Targeting Technology: Physical, Chemical, and Biological Methods (Schreier, 2001); Biomedical Aspects of Drug Targeting (Muzykantov et al., 2002); Liposomes: A Practical Approach (Torchilin et al., 2003); Cellular Drug Delivery: Principles and Practice (Lu et al., 2004); and Protein-Protein Interactions: A Molecular Cloning Manual (Golemis et al., 2005).

Patents identifying and describing specific targeting ligands of medical importance and methods for their use, include, but are not limited to U.S. Pat. Nos. 5,128,326; 5,580,960; 5,610,031; 5,625,040; 5,648,465; 5,766,922; 5,770,565; 5,792,743; 5,849,865; 5,866,165; 5,872,231; 6,121,231; 6,140,117; 6,159,467; 6,204,054; 6,352,972; and 6,482,410. The disclosures of each of the publications and patents in paragraphs [0196] and [0197] are hereby incorporated by reference herein in their entirety for all purposes.

Briefly, many different targeting ligands can be selected to bind to specific domains of various adhesion molecules (e.g., the immunoglobulin Superfamily [ICAM 1-3, PECAM-1, VCAM-1], the Selectins [EWLAM-1, LECAM-1, GMP-140] and the Integrins [LEA-1]). Targeting ligands in this regard may include (1) lectins, (2) a wide variety of carbohydrate or sugar moieties, (3) antibodies, (4) antibody fragments, (5) Fab fragments, such as, for example, Fab′₂; and (6) synthetic peptides, including, for example, Arginine-Glycine-Aspartic Acid (R-G-D) which may be targeted to wound healing. While many of these materials may be derived from natural sources, some may be synthesized by molecular biological recombinant techniques, and others may be synthetic in origin. Peptides may be prepared by a variety of techniques known in the art. Targeting ligands derived or modified from human leukocyte origin (e.g., CD11a/CD 18 and leukocyte cell surface glycoprotein [LFA-1]) may also be used, as these bind to the endothelial cell receptor ICAM-1. The cytokine inducible member of the immunoglobulin superfamily, VCAM-1, which is mononuclear leukocyte-selective, may also be used as a targeting ligand. VLA-4, derived from human monocytes, may be used to target VCAM-1. Antibodies and other targeting ligands may be employed to target endoglin, which is an endothelial cell proliferation marker. Endoglin is upregulated on endothelial cells in miscellaneous solid tumors. Further, the cadherin family of cell adhesion molecules may also be used as targeting ligands including, for example, (1) the E-, N-, and P-cadherins; (2) cadherin-4, (3) cadherin-5, (4) cadherin-6, (5) cadherin-7, (6) cadherin-8, (7) cadherin-9, (8) cadherin-10, (9) cadherin-11, and, most preferably, (11) cadherin C-5. Further, antibodies directed to cadherins, may be used to recognize cadherins expressed locally by specific endothelial cells.

Targeting ligands may be selected for targeting antigens, including antigens associated with breast cancer, such as epidermal growth factor receptor (EGFR), fibroblast growth factor receptor, erbB2/BER-2, and tumor associated carbohydrate antigens, CTA 16.88, homologous to cytokeratins 8, 18, and 19, which is expressed by most epithelial-derived tumors including carcinomas of the colon, pancreas, breast, and ovary. Chemically conjugated bispecific anti-cell surface antigen, and anti-hapten Fab′-Fab antibodies may also be used as targeting ligands. The MG series monoclonal antibodies may be selected for targeting (e.g., gastric cancer). Fully humanized antibodies or antibody fragments are most preferred.

There are a variety of cell surface epitopes on epithelial cells for which targeting ligands may be selected. For example, the human papilloma virus (HPV) has been associated with benign and malignant epithelial proliferations in both skin and mucosa. Two HPV oncogenic proteins, E6 and E7, may be targeted, as these may be expressed in certain epithelial-derived cancers (e.g., as cervical carcinoma). Membrane receptors for peptide growth factors (PGF-R), which are involved in cancer cell proliferation, may also be selected as tumor antigens. Also, epidermal growth factor (EGF) and interleukin-2 may be targeted with suitable targeting ligands, including peptides, which bind these receptors. Certain melanoma-associated antigens (MAAs) (e.g., epidermal growth factor receptor [EGFR]), and adhesion molecules expressed by, for example, malignant melanoma cells can also be targeted with specific ligands.

A wide variety of targeting ligands may be selected for targeting myocardial cells. Exemplary targeting ligands include, for example, anticardiomyosin antibody, which may comprise polyclonal antibody, Fab′₂ fragments, or be of human origin, animal origin, for example, mouse or of chimeric origin. Again, in all antibody and antibody fragment embodiments, fully humanized species are preferred. Additional targeting ligands include (1) dipyridamole, digitalis, nifedipine, and apolipoprotein; (2) low-density lipoproteins (LDL) including vLDL and methyl LDL; (3) ryanodine, endothelin, complement receptor type 1, IgG Fc, beta 1-adrenergic, dihydropyridine, adenosine, mineralocorticoid, nicotinic acetylcholine and muscarinic acetylcholine; antibodies to the human alpha 1A-adrenergic receptor; bioactive agents, such as drugs, including the α-1-antagonist prazosin; (4) antibodies to the anti-beta-receptor, and drugs which bind to the anti-beta-receptor; (5) anti-cardiac RyR antibodies; (6) endothelin-1, which is an endothelial cell-derived vasoconstrictor peptide that exerts a potent positive inotropic effect on cardiac tissue (endothelin-1 binds to cardiac sarcolemmal vesicles); (7) monoclonal antibodies which may be generated to the T-cell receptor-β receptor, and thereby employed to generate targeting ligands; (8) the complement inhibitor sCR1; (9) drugs, peptides, or antibodies which are generated to the dihydropyridine receptor; and (10) monoclonal antibodies directed toward the anti-interleukin-2 receptor, which may be used as targeting ligands to direct the nanocarriers of this specification to areas of myocardial tissue which express this receptor and which may be upregulated in conditions such as inflammation.

In another embodiment, the targeting ligands are directed to lymphocytes which may be T-cells or B-cells. Depending on the targeting ligand, the composition may be targeted to one or more classes or clones of T-cells. To select a class of targeted lymphocytes, a targeting ligand having specific affinity for that class is employed. For example, an anti CD-4 antibody can be used for selecting the class of T-cells harboring CD4 receptors, an anti CD-8 antibody can be used for selecting the class of T-cells harboring CD-8 receptors, an anti CD-34 antibody can be used for selecting the class of T-cells harboring CD-34 receptors, etc. A lower molecular weight ligand is preferably employed (e.g., Fab or a peptide fragment). For example, an OKT3 antibody or OKT3 antibody fragment may be used. When a receptor for a class of T-cells is selected, or clones of T-cells are selected, the composition will be delivered to that class of cells. Using HLA-derived peptides, for example, will allow selection of targeted clones of cells expressing reactivity to HLA proteins. Another major area for targeted delivery involves the interlekin-2 (IL-2) system. IL-2 is a T-cell growth factor produced following antigen- or mitogen-induced stimulation of lymphoid cells. Among the cell types which produce IL-2 are CD4⁺ and CD8⁺ T-cells, large granular lymphocytes, as well as certain T-cell tumors, etc. Still other systems which can be used in embodiments of the present invention include IgM-mediated endocytosis in B-cells or a variant of the ligand-receptor interactions described above, wherein the T-cell receptor is CD2 and the ligand is lymphocyte function-associated antigen 3 (LFA-3).

Synthetic compounds, which combine a natural amino acid sequence with synthetic amino acids, can also be used as targeting ligands as well as peptides, or derivatives thereof. In view of the present disclosure, as will be immediately apparent to those skilled in the art, a large number of additional targeting ligands may be used with the present teachings, in addition to those exemplified above. Other suitable targeting ligands include, for example, conjugated peptides, such as, for example, glycoconjugates and lectins, which are peptides attached to sugar moieties. The compositions may comprise a single targeting ligand, as well as two or more different targeting ligands.

Preferred embodiments of present invention include the use of magnetic targeting of nanocarriers and/or contrast agents. This is accomplished by using magnetically susceptible compositions bound to, and/or associated with said nanocarriers or contrast agents exteriorly, and/or by other means, and then guided by a magnetic field. A wide variety of sources are known in the art describing techniques, procedures, and methods for active targeting of drug-containing vesicles using magnetically susceptible materials, and said materials may be hereafter referred to as “magnetic compositions” or “magnetic targeting components.” For enablement and other purposes, preferred publications include, but are not limited to Principles of Nuclear Magnetism (International Series of Monographs on Physics) (Abragam, 1983); Ultrathin Magnetic Structures I: An Introduction to the Electronic, Magnetic and Structural Properties (Bland et al., 1994); Magnetism: Molecules to Materials, (Miller et al., 2001); and Drug and Enzyme Targeting, Parts: Volume 112: Drug and Enzyme Targeting (Methods in Enzymology) (Colowick et al., 2006). Important peer-reviewed research publications associated with magnetic targeting and drug delivery include Widder et al., (1979); Hsieh et al., (1981); Kost et al., (1987); He et al., (1993); Wu et al., (1993); Wu et al., (1994); Chen et al., (1997); Rudge et al., (2000); Jones et al., (2001); Lubbe et al., (2001); and Moroz et al., (2001). Patents concerning magnetic targeting and drug delivery relevant to the teachings of this specification include U.S. Pat. Nos. 6,200,547; 6,482,436; 6,488,615; and 6,663,555. Each of the publications and patents listed in this paragraph [0204] are hereby incorporated by reference herein in their entirety for all purposes.

Magnetic targeting components for use with embodiments of the present invention are typically comprised of 1% to 70% of a biocompatible polymer, and 30% to 99% of a magnetic component. With compositions having less than 1% polymer, the physical integrity of the particle is less than optimal. With compositions of greater than 70% polymer, the magnetic susceptibility of the particle is generally reduced beyond an optimal level for the nanocarriers described herein. The compositions may be of any shape, different shapes conferring differing advantageous properties, with an average size of approximately 0.1 μm to approximately 30 μm in diameter.

Said magnetic targeting components have the general properties of having Curie temperatures (Tc) greater than the normal human body temperature (37° C.), having high magnetic saturation (>approximately 20 Am²/kg), and being ferromagnetic or ferrimagnetic. Examples of suitable magnetic components include magnetic iron sulfides such as pyrrhotite (Fe₇S₈), and greigite (Fe₄S₄); magnetic ceramics such as Alnico 5, Alnico 5 DG, Sm₂CO₁₇, SmCo₅, and NdFeB; magnetic iron alloys, such as jacobsite (MnFe₂O₄), trevorite (NiFe₂O₄), awaruite (Ni₃Fe), and wairauite (CoFe); and magnetic metals such as metallic iron (Fe), cobalt (Co), and nickel (Ni). Each of the magnetic components can have added to its chemical formula specific impurities that may or may not alter the magnetic properties of the material. Doped ferromagnetic or ferrimagnetic materials, within the above limits of Curie temperatures and magnetic saturation values, are considered to be suitable as magnetic compositions for use in active targeting of the nanocarriers described herein. Specifically excluded from suitable magnetic components and the magnetically susceptible compositions are the iron oxides magnetite (Fe₃O₄), hematite (α-Fe₂O₃), and maghemite (γ-Fe₂O₃).

The term “metallic iron” indicates that iron is primarily in its “zero valence” state (Fe⁰). Generally, the metallic iron is greater than approximately 85% Fe⁰, and preferably greater than approximately 90% Fe⁰. More preferably, the metallic iron is greater than approximately 95% “zero valence” iron. Metallic iron is a material with high magnetic saturation and density (i.e., 218 emu/gm and 7.8 gm/cm³), which are much higher than magnetite (i.e., 92 emu/gm and 5.0 gm/cm³). The density of metallic iron is 7.8 gm/cm³, while magnetite is approximately 5.0 gm/cm³. Thus, the magnetic saturation of metallic iron is approximately 4-fold higher than that of magnetite per unit volume (Craik, 1995). The use of said magnetic compositions with the nanocarriers of this specification results in magnetically responsive compositions with a high degree of magnetic saturation (i.e., >50 emu/gm). The higher magnetic saturation allows the nanocarrier with biologically active agents (e.g., therapeutic macromolecules) to be effectively targeted to the desired site by an external magnetic field, and eventually be extravasated through blood vessel walls, penetrating into the target tissues of the patient.

The biocompatible polymers for use with said magnetic targeting components may be bioinert and/or biodegradable. Some nonlimiting examples of biocompatible polymers are polylactides, polyglycolides, polycaprolactones, polydioxanones, polycarbonates, polyhydroxybutyrates, polyalkylene oxalates, polyanhydrides, polyamides, polyacrylic acid, poloxamers, polyesteramides, polyurethanes, polyacetals, polyorthocarbonates, polyphosphazenes, polyhydroxyvalerates, polyalkylene succinates, poly(malic acid), poly(amino acids), alginate, agarose, chitin, chitosan, gelatin, collagen, atelocollagen, dextran, proteins, polyorthoesters, copolymers, terpolymers, and combinations and/or mixtures thereof. Said biocompatible polymers can be prepared in the form of matrices, which are polymeric networks. One type of polymeric matrix is a hydrogel, which can be defined as a water-containing polymeric network. The polymers used to prepare hydrogels can be based on a variety of monomer types, such as those based on methacrylic and acrylic ester monomers, acrylamide (methacrylamide) monomers, and N-vinyl-2-pyrrolidone. Hydrogels can also be based on polymers such as starch, ethylene glycol, hyaluran, chitose, and/or cellulose. To form a hydrogel, monomers are typically crosslinked with cross-linking agents such as ethylene dimethacrylate, N,N-methylenediacrylamide, methylenebis(4-phenyl isocyanate), epichlarohydin glutaraldehyde, ethylene dimethacrylate, divinylbenzene, and allyl methacrylate. Hydrogels can also be based on polymers such as starch, ethylene glycol, hyaluran, chitose, and/or cellulose. In addition, hydrogels can be formed from a mixture of monomers and polymers.

Another type of polymeric network can be formed from more hydrophobic monomers and/or macromers. Matrices formed from these materials generally exclude water. Polymers used to prepare hydrophobic matrices can be based on a variety of monomer types such as alkyl acrylates and methacrylates, and polyester-forming monomers such as ε-caprolactone, glycolide, lactic acid, glycolic acid, lactide, and the like. When formulated for use in an aqueous environment, these materials do not normally need to be cross-linked, but they can be cross-linked with standard agents such as divinyl benzene. Hydrophobic matrices may also be formed from reactions of macromers bearing the appropriate reactive groups such as the reaction of diisocyanate macromers with dihydroxy macromers, and the reaction of diepoxy-containing macromers with dianhydride or diamine-containing macromers.

The biocompatible polymers for use with said magnetic compositions can be preferably prepared in the form of dendrimers and/or hyperbranched polymers. The size, shape, and properties of these dendrimers can be molecularly tailored to meet specialized end uses, such as a means for the delivery of high concentrations of carried material per unit of polymer, controlled delivery, targeted delivery, and/or multiple species delivery or use. The dendritic polymers can be prepared according to methods known in the art, including those detailed herein. The biocompatible polymers for use with said magnetic compositions may be, for example, biodegradable, bioresorbable, bioinert, and/or biostable. Bioresorbable hydrogel-forming polymers are generally naturally occurring polymers such as polysaccharides, examples of which include, but are not limited to hyaluronic acid, starch, dextran, heparin, and chitosan; and proteins—and other polyamino acids—examples of which include but are not limited to gelatin, collagen, fibronectin, laminin, albumin, and active peptide domains thereof. Matrices formed from these materials degrade under physiological conditions, generally via enzyme-mediated hydrolysis.

Bioresorbable matrix-forming polymers are generally synthetic polymers prepared via condensation polymerization of one or more monomers. Matrix-forming polymers of this type include: polylactide (PLA), polyglycolide (PGA), polylactide coglycolide (PLGA), polycaprolactone (PCL), and copolymers of these materials, polyanhydrides, and polyortho esters. Biostable or bioinert hydrogel matrix-forming polymers are generally synthetic or naturally occurring polymers which are soluble in water, matrices of which are hydrogels or water-containing gels. Examples of this type of polymer include: polyvinylpyrrolidone (PVP), polyethylene glycol (PEG), polyethylene oxide (PEO), polyacrylamide (PAA), polyvinyl alcohol (PVA), and the like. Biostable or bioinert matrix-forming polymers are generally synthetic polymers formed from hydrophobic monomers such as methyl methacrylate, butyl methacrylate, dimethyl siloxanes, and the like. These polymer materials generally do not possess significant water solubility, but can be formulated as neat liquids which form strong matrices upon activation. Said polymers may also contain hydrophilic and hydrophobic monomers.

Targeting moieties may also be incorporated into the nanocarriers of this specification in a variety of other ways. Additional preferred methods include being associated covalently or non-covalently with one or more of the polymers described herein. Photopolymerizable elements are most preferred, and photopolymerization may be used in cross-linking the targeting moieties with themselves, and/or linking said moieties to the nanocarriers. In other preferred embodiments, the targeting moiety is covalently bound to the surface of the nanocarrier by a spacer including, for example, hydrophilic polymers, preferably polyethylene glycol. Preferred molecular weights of the polymers are from 1,000 Daltons to 10,000 Daltons, with 500 Daltons being most preferred. Preferably, the polymer is bifunctional with the targeting moiety bound to a terminus of the polymer. Generally, in the case of a targeting ligand, it should range from approximately 0.1 to approximately 20 mole % of the exterior components of the vesicle. The exact ratio will depend on the particular targeting ligand and the application.

Exemplary covalent bonds by which the targeting moieties are associated with the nanocarriers of embodiments of the present invention include, for example, amide (—CONH—); thioamide (—CSNH—); ether (ROR′), where R and R′ may be the same or different and are other than hydrogen; ester (—COO—); thioester (—COS—); —O—; —S—; —Sₙ, where n is greater than 1, preferably approximately 2 to approximately 8, and more preferably approximately 2; carbamates; —NH—; —NR—, where R is alkyl, for example, alkyl of from 1 carbon to approximately 4 carbons; urethane; and substituted imidate; and combinations of two or more of these. Covalent bonds between targeting ligands and polymers may be achieved through the use of molecules that may act as spacers to increase the conformational and topographical flexibility of the ligand. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like. In addition, in the case of targeting ligands which comprise peptide moieties, side-chain-to-side-chain cross-linking may be complemented with side chain-to-end cross-linking and/or end-to-end cross-linking. Also, small spacer molecules, such as dimethylsuberimidate, may be used to accomplish similar objectives. The use of agents, including those used in Schiff's base-type reactions, such as gluteraldehyde, may also be employed.

The covalent linking of targeting moieties to embodiments of the invention may also be accomplished using synthetic organic techniques, which in view of the present disclosure, will be readily apparent to one of ordinary skill in the art. For example, the targeting moieties may be linked to the materials, including the polymers, via the use of well-known coupling or activation agents. As known to those skilled in the art, activating agents are generally electrophilic, which can be employed to elicit the formation of a covalent bond. Exemplary activating agents which may be used include, for example, carbonyldiimidazole (CDI), dicyclohexylcarbodiimide (DCC), diisopropylcarbodiimide (DIC), methyl sulfonyl chloride, Castro's Reagent, and diphenyl phosphoryl chloride. The covalent bonds may involve cross-linking and/or polymerization. Cross-linking preferably refers to the attachment of two chains of polymer molecules by bridges, composed of either an element, a group, or a compound, which join certain carbon atoms of the chains by covalent chemical bonds. For example, cross-linking may occur by photopolymerization and for polypeptides which are joined by the disulfide bonds of the cysteine residue. Cross-linking may be achieved, for example, by (1) adding a chemical substance (e.g., cross-linking agent) and exposing the mixture to heat, or (2) subjecting a polymer to high-energy radiation. A variety of cross-linking agents, or “tethers,” of different lengths and/or functionalities are described (Hermanson, 1996), the disclosures of which are hereby incorporated herein by reference in their entirety for all purposes.

### Contrast Agents

Most of the contrast agents for use with preferred embodiments of this specification have a high degree of echogenicity, the ability of an object to reflect ultrasonic waves. Thus, preferred contrast agents comprise small, stabilized gas-filled microbubbles that can pass through the smallest capillaries of the patient. For imaging purposes, the larger the microbubbles, the better the acoustic responsiveness; however, if the bubbles are too large, they will be retained in the capillaries of the patient and they are unable to cross the pulmonary circulation. Further, the size of said contrast agents will likely vary depending upon the drug delivery application. Properties of the ideal contrast agent for use with embodiments of this disclosure (1) are nontoxic and easily eliminated by the patient, (2) are administered intravenously, (3) pass easily through the microcirculation, (4) are physically stable, and (5) are acoustically responsive with stable harmonics and the capability of rapid disruption. The ultrasonic characteristics of these contrast agents depend not only on the size of the bubble, but also on the composition of the shell and the gas contained therein. The outer shell of preferred microbubbles is composed of many different substances including albumin, polymers, palmitic acid, or phospholipids. The composition of the shell determines its elasticity, its behavior in an ultrasonic field, how rapidly the bubble is taken up by the immune system, and the physiological methods for metabolism and elimination from the patient. A more hydrophilic material tends to be taken up more easily, which reduces the microbubble residence time in the circulation. In general, the stiffer the shell, the more easily it will crack or break when exposed to ultrasonic energy. Conversely, the more elastic the shell, the greater its ability to be compressed or resonated, a characteristic of considerable importance for the preferred contrast agents used in the practice of the present teachings.

The gas core is a critical component of the microbubble because it is the primary determinate of its echogenicity. When gas bubbles are caught in an ultrasonic frequency field, they compress, oscillate, and reflect a characteristic echo, which generates the strong and unique sonogram in contrast-enhanced ultrasound. Gas cores can be composed of air, nitrogen or, for example, heavy gases like perfluorocarbon. Heavy gases are less water-soluble so they are less likely to leak out from the microbubble to impair echogenicity. Thus, microbubbles with heavy gas cores are likely to remain in the circulation longer.

Ultrasound contrast reagents for use with the present invention can be prepared as described in U.S. Pat. No. 6,146,657, the disclosures of which are hereby incorporated herein by reference in their entirety for all purposes. Briefly, a sealed container is used comprising an aqueous lipid suspension phase and a substantially separate gaseous phase. Prior to use, the container and its contents may be agitated, causing the lipid and gas phases to mix, resulting in the formation of gas-filled liposomes which entrap the gas. The resulting gas-filled liposomes provide an excellent contrast enhancement agent for diagnostic imaging, particularly using ultrasound or magnetic resonance imaging, and for assisting in acoustically mediated intracellular therapeutic delivery in vivo, as detailed herein. A wide variety of lipids may be employed in the aqueous lipid suspension phase of the preferred contrast agents for use with the present teachings. The lipids may be saturated or unsaturated, and may be in linear or branched form, as desired. Such lipids may comprise, for example, fatty acids molecules that contain a wide range of carbon atoms, preferably between approximately 12 carbon atoms and 22 carbon atoms. Hydrocarbon groups consisting of isoprenoid units, prenyl groups, and/or sterol moieties (e.g., cholesterol, cholesterol sulfate, and analogs thereof) may also be employed. The lipids may also bear polymer chains, such as the amphipathic polymers polyethylene glycol (PEG), polyvinylpyrrolidone (PVP), derivatives thereof for in vivo targeting; charged amino acids such as polylysine or polyarginine, for binding of a negatively charged compound; carbohydrates, for in vivo targeting, such as described in U.S. Pat. No. 4,310,505; glycolipids, for in vivo targeting; or antibodies and other peptides and proteins, for in vivo targeting, etc., as desired. Such targeting or binding compounds may be simply added to the aqueous lipid suspension phase or may be specifically chemically attached to the lipids using methods described herein, or employing other methodologies known in the art. The lipids may also be anionic or cationic.

Classes of and specific lipids for use as shell materials with preferred contrast agents for use with the present teachings, but are not limited to, include phosphatidylcholines such as dioleoylphosphatidylcholine, dimyristoylphosphatidylcholine, dipalmitoylphosphatidylcholine (DPPC), and distearoylphosphatidylcholine; phosphatidylethanolamines such as dipatmitoylphosphatidylethanolamine (DPPE), dioleoylphosphatidylethanolamine, and N-succinyl-dioleoylphosphatidylethanolamine; phosphatidylserines, phosphatidylglycerols, and sphingolipids; glycolipids such as ganglioside GM1; glucolipids, sulfatides, and glycosphingolipids; phosphatidic acids such as dipalmatoylphosphatidic acid (DPPA); palmitic fatty acids, stearic fatty acids, arachidonic fatty acids, lauric fatty acids, myristic fatty acids, lauroleic fatty acids, physeteric fatty acids, myristoleic fatty acids, palmitoleic fatty acids, petroselinic fatty acids, oleic fatty acids, isolauric fatty acids, isomyristic fatty acids, isopalmitic fatty acids, and isostearic fatty acids; cholesterol and cholesterol derivatives such as cholesterol hemisuccinate, cholesterol sulfate, and cholesteryl-(4′-trimethylammonio)-butanoate; polyoxyethylene fatty acid esters, polyoxyethylene fatty acid alcohols, polyoxyethylene fatty acid alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene fatty acid polymers, polyoxyethylene fatty acid stearates, 12-(((7′-diethylaminocoumarin-3-yl)-carbonyl)-methylamino)-octadecanoic acid, N-(12-(((7′-diethylamino-coumarin-3-yl)-carbonyl)-methyl-amino)octadecanoyl)-2-amino-palmitic acid, 1,2-dioleoyl-sn-glycerol, 1,2-dipalmitoyl-sn-3-succinylglycerol, 1,3-dipalmitoyl-2-succinyl-glycerol, 1-hexadecyl-2-patmitoyl-glycerophosphoethanolamine, and palmitoylhomocysteine, lauryltrimethylammonium bromide (lauryl-=dodecyl-); cetyltrimethylammonium bromide (cetryl-=hexadecyl-), myristyltrimethylammonium bromide (myristyl-=tetradecyl-); alkyldimethylbenzylammonium chlorides such as wherein alkyl is a C₁₂, C₁₄, or C₁₆ alkyl; and benzyldimethyldodecylammonium bromide, benzyldimethyldodecylammonium chloride, benzyldimethylhexadecylammonium bromide, benzyldimethylhexadecylammonium chloride, benzyldimethyltetradecylammonium bromide, benzyldimethyltetradecylammonium chloride, cetyldimethylethylammonium bromide, cetyldimethylethylammonium chloride, cetylpyridinium bromide, cetylpyridinium chloride, N-(1-2,3-dioleoyloxy)-propyl)-N,N,N-trimethylammonium chloride (DOTMA), 1,2-dioleoyloxy-3-(trimethylammonio)propane (DOTAP), and 1,2-dioleoyl-e-(4′-trimethylammonio)-butanoyl-sn-glycerol (DOTB).

In addition, the aqueous lipid phase may further comprise a polymer, preferably an amphipathic polymer, and preferably one that is directly bound (i.e., chemically attached) to the lipid. Preferably, the amphipathic polymer is polyethylene glycol or a derivative thereof. The most preferred combination is the lipid dipatmitoylphosphatidylethanolamine (DPPE) bound to polyethylene glycol (PEG), especially PEG of an average molecular weight of approximately 5000 (DPPE-PEG5000). The PEG or other polymer may be bound to the DPPE or other lipid through a covalent linkage such as through an amide, carbamate, or amine linkage. Alternatively, ester, ether, thioester, thioamide, or disulfide (i.e., thioester) linkages may be used with the PEG or other polymer to bind the polymer to, for example, cholesterol or other phospholipids. A particularly preferred combination of lipids is DPPC, DPPE-PEG5000, and DPPA, especially in a ratio of approximately 82:8:10% (mole %), DPPC: DPPE-PEG5000:DPPA.

Examples of classes of and specific suitable gases for use as the core of preferred contrast agents, suitable for use with the present teachings, are those gases that are substantially insoluble in an aqueous shell suspension. Suitable gases that are substantially insoluble or soluble include, but are not limited to hexafluoroacetone, isopropylacetylene, allene, tetrafluoroallene, boron trifluoride, 1,2-butadiene, 1,3-butadiene, 1,2,3-trichlorobutadiene, 2-fluoro-1,3-butadiene, 2-methyl-1,3 butadiene, hexafluoro-1,3-butadiene, butadiyne, 1-fluorobutane, 2-methylbutane, decafluorobutane (perfluorobutane), decafluoroisobutane (perfluoroisobutane), 1-butene, 2-butene, 2-methyl-1-butene, 3-methyl-1-butene, per fluoro-1-butene, per fluoro-1-butene, perfluoro-2-butene, 4-phenyl-3-butene-2-one, 2-methyl-1-butene-3-yne, butylnitrate, 1-butyne, 2-butyne, 2-chloro-1,1,1,4,4,4-hexafluoro-butyne, 3-methyl-1-butyne, perfluoro-2-butyne, 2-bromo-butyraldehyde, carbonyl sulfide, crotononitrile, cyclobutane, methylcyclobutane, octafluorocyclobutane (perfluorocyclobutane), perfluoroisobutane, 3-chlorocyclopentene, cyclopropane, 1,2-dimethylcyclopropane, 1,1-dimethylcyclopropane, ethyl cyclopropane, methylcyclopropane, diacetylene, 3-ethyl-3-methyldiaziridine, 1,1,1-trifluorodiazoethane, dimethylamine, hexafluorodimethylamine, dimethylethylamine, bis-(dimethyl phosphine) amine, 2,3-dimethyl-2-norbornane, perfluorodimethylamine, dimethyloxonium chloride, 1,3-dioxolane-2-one, 1,1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoroethane, 2-chloro-1,1-difluoroethane, chloroethane, chloropentafluoroethane, dichlorotrifluoroethane, fluoroethane, nitropentafluoroethane, nitrosopentafluoro-ethane, perfluoroethane, perfluoroethylamine, ethyl vinyl ether, 1,1-dichloroethylene, 1,1-dichloro-1,2-difluoro-ethylene, 1,2-difluoro-ethylene, methane, methane-sulfonyl-chloride-trifluoro, methane-sulfonyl-fluoride-trifluoro, methane-(penta-fluorothio)trifluoro, methane-bromo-difluoro-nitroso, methane-bromo-fluoro, methane-bromo-chloro-fluoro, methane-bromo-trifluoro, methane-chloro-difluoro-nitro, methane-chloro-dinitro, methane-chloro-fluoro, methane-chloro-trifluoro, methane-chloro-difluoro, methane-dibromo-difluoro, ethane-dichloro-difluoro, methane-dichloro-fluoro, methane-difluoro, methane-difluoro-iodo, methane-disilano, methane-fluoro, methane-iodonethane-iodo-trifluoro, methane-nitro-trifluoro, methane-nitroso-trifluoro, methane-tetrafluoro, methane-trichloro-fluoro, methane-trifluoro, methanesulfenylchloride-trifluoro, 2-methyl butane, methyl ether, methyl isopropyl ether, methyl lactate, methyl nitrite, methyl sulfide, methyl vinyl ether, neopentane, nitrogen (N₂), nitrous oxide, 1,2,3-nonadecane tricarboxylic acid-2-hydroxytrimethylester, 1-nonene-3-yne, oxygen (O₂), oxygen 17 (¹⁷O₂), 1,4-pentadiene, n-pentane, dodecafluoropentane (perfluoropentane), tetradecafluorohexane (perfluorohexane), perfluoroisopentane, perfluoroneopentane, 2-pentanone-4-amino-4-methyl, 1-pentene, 2-pentene {cis #0}, 2-pentene {trans}, 1-pentene-3-bromo, 1-pentene-perfluoro, phthalic acid-tetrachloro, piperidine-2,3,6-trimethyl, propane, propane-1,1,1,2,2,3-hexafluoro, propane-1,2-epoxy, propane-2,2 difluoro, propane-2-amino, propane-2-chloro, propane-heptafluoro-1-nitro, propane-heptafluoro-1-nitroso, perfluoropropane, propene, propyl-1,1,1,2,3,3-hexafluoro-2,3 dichloro, propylene-1-chloro, propylene-chloro-{trans}, propylene-2-chloro, propylene-3-fluoro, propylene-perfluoro, propyne, propyne-3,3,3-trifluoro, styrene-3-fluoro, sulfur hexafluoride, sulfur (di)-decafluoro(S₂F₁₀), toluene-2,4-diamino, trifluoroacetonitrile, trifluoromethyl peroxide, trifluoromethyl sulfide, tungsten hexafluoride, vinyl acetylene, vinyl ether, neon, helium, krypton, xenon (especially rubidium enriched hyperpolarized xenon gas), carbon dioxide, helium, and air. Fluorinated gases (i.e., a gas containing one or more fluorine molecules such as sulfur hexafluoride); fluorocarbon gases (i.e., a fluorinated gas which is a fluorinated carbon or gas); and perfluorocarbon gases (i.e., a fluorocarbon gas which is fully fluorinated such as perfluoropropane and perfluorobutane) are preferred.

A targeted contrast agent, for use with embodiments of the present invention, is a contrast agent that can bind selectively or specifically to a desired target. The same aforementioned preferred shell materials and preferred gases may be used in preferred targeted contrast agents, with the addition of a targeting moiety, as described in detail herein, or other structure, either alone or in combination. For example, an antibody fragment or an aptamer may be bound to the surface of said contrast agent by the methods described herein and/or in the art. If an antibody or similar targeting mechanism is used, selective or specific binding to a target can be determined based on standard antigen/epitope/antibody complementary binding relationships. Further, other controls may be used. For example, the specific or selective targeting of the microbubbles can be determined by exposing targeted microbubbles to a control tissue, which includes all of the components of the test tissue except for the desired target ligand, epitope, or other structure.

Specific or selectively targeted contrast agents can be produced by methods known in the art. For example, targeted contrast agents can be prepared as perfluorocarbon or other gas-filled microbubbles with a monoclonal antibody on the shell as a ligand for binding to a target ligand in a patient as described in Villanueva et al. (1998); the disclosures of which are hereby incorporated herein by reference in their entirety for all purposes. For example, perfluorobutane can be dispersed by sonication in an aqueous medium containing phosphatidylcholine, a surfactant, and a phospholipid derivative containing a carboxyl group. The perfluorobutane is encapsulated during sonication by a lipid shell. The carboxylic groups are exposed to an aqueous environment and used for covalent attachment of antibodies to the microbubbles by the following steps. First, unbound lipid dispersed in the aqueous phase is separated from the gas-filled microbubbles by flotation. Second, carboxylic groups on the microbubble shell are activated with 1-ethyl-3-(3-dimethylaminopropyl) carbodimide, and antibody is then covalently attached via its primary amino groups with the formation of amide bonds.

Targeted microbubbles can also be prepared with a biotinylated shell, using the methods described in Weller et al., 2002, the disclosures of which are hereby incorporated herein by reference in their entirety for all purposes. For example, lipid-based perfluorocarbon-filled microbubbles can be prepared with monoclonal antibody on the shell using avidin-biotin bridging chemistry, employing, for example, the following protocol. Perfluorobutane is dispersed by sonication in aqueous saline containing phosphatidyl choline, polyethylene glycol (PEG) stearate, and a biotinylated derivative of phosphatidylethanolamine, as described in the art. The sonication results in the formation of perfluorobutane microbubbles coated with a lipid monolayer shell and carrying the biotin label. Antibody conjugation to the shell is achieved via avidin-biotin bridging chemistry. Samples of biotinylated microbubbles are washed in phosphate-buffered saline (PBS) by centrifugation to remove the lipid not incorporated in the microbubble shell. Next, the microbubbles are incubated in a solution (0.1-10 μg/ml) of streptavidin in PBS. Excess streptavidin is removed by washing with PBS. The microbubbles are then incubated in a solution of biotinylated monoclonal antibody in PBS and washed. The resultant microbubble has antibody conjugated to the lipid shell via biotin-streptavidin-biotin linkage. In another example, biotinylated microbubbles can be prepared by sonication of an aqueous dispersion of decafluorobutane gas, distearoylphosphatidylcholine, polyethyleneglycol-(PEG)-state, and distearoyl-phosphatidylethanolamine-PEG-biotin. Microbubbles can then be combined with streptavidin, washed, and combined with biotinylated echistatin.

Targeted microbubbles can also be prepared with an avidinated shell, as is known in the art. In a preferred embodiment, a polymer microbubble can be prepared with an avidinated or streptavidinated shelf. In another preferred embodiment, avidinated microbubbles can be used by the methods disclosed herein. When using avidinated microbubbles, a biotinylated antibody or fragment thereof or another biotinylated targeting molecule or fragments thereof can be administered to the patient. For example, a biotinylated targeting ligand (e.g., an antibody, protein, or other bioconjugate) can be used. Thus, a biotinylated antibody, targeting ligand or molecule, or fragment thereof can bind to a desired target within the patient. Once bound to the desired target, the contrast agent with an avidinated shell can bind to the biotinylated antibody, targeting molecule, or fragment thereof. An avidinated contrast agent can also be bound to a biotinylated antibody, targeting ligand or molecule, or fragment thereof, prior to administration. When using a targeted contrast agent with a biotinylated shell or an avidinated shell, a targeting ligand or molecule can be administered to the patient. For example, a biotinylated targeting ligand such as an antibody, protein, or other bioconjugate, can be administered to the patient and allowed to accumulate at a target site. A fragment of the targeting ligand or molecule can also be used. When a targeted contrast agent with a biotinylated shell is used, an avidin linker molecule, which attaches to the biotinylated targeting ligand, can be administered to the patient. Then a targeted contrast agent with a biotinylated shell is administered. The targeted contrast agent binds to the avidin linker molecule, which is bound to the biotinylated targeting ligand, which is itself bound to the desired target. In this way, a three-step method can be used to target contrast agents to a desired target.

Targeted contrast agents or nontargeted contrast agents can also comprise a variety of markers, detectable moieties, or labels. Thus, a microbubble contrast agent, equipped with a targeting ligand or antibody incorporated into the shell of the microbubble, can also include another detectable moiety or label. As used herein, the term “detectable moiety” is intended to mean any suitable label, including, but not limited to enzymes, fluorophores, biotin, chromophores, radioisotopes, colored particles, electrochemical, chemical-modifying, or chemiluminescent moieties. Common fluorescent moieties include fluorescein, cyanine dyes, coumarins, phycoerythrin, phycobiliproteins, dansyl chloride, Texas Red, and lanthanide complexes. Of course, the derivatives of these compounds, which are known to those skilled in the art, are also included as common fluorescent moieties. The detection of the detectable moiety can be direct, provided that the detectable moiety is itself detectable such as, for example, in the case of fluorophores. Alternatively, the detection of the detectable moiety can be indirect. In the latter case, a second moiety reactable with the detectable moiety, itself being directly detectable, can be employed. The detectable moiety may be inherent to the molecular probe. For example, the constant region of an antibody can serve as an indirect detectable moiety to which a second antibody having a direct detectable moiety can specifically bind. Targeted contrast agents can also be modified by allowing larger bubbles to separate in solution relative to smaller bubbles. For example, targeted contrast agents can be modified by allowing larger bubbles to float higher in solution relative to smaller bubbles. A population of microbubbles, of an appropriate size to achieve a desired volume percentage, can subsequently be selected. Other means are available in the art for separating micron-sized and nano-sized particles, and could be adapted to select a microbubble population of the desired volume of submicron bubbles such as, for example, by centrifugation. Sizing of the microbubbles can occur before or after the microbubbles are adapted for targeting.

The targeted contrast agents may be used with the nanocarriers of this disclosure by targeting said contrast agents to a variety of cells, cell types, antigens, cellular membrane proteins, organs, markers, tumor markers, angiogenesis markers, blood vessels, thrombus, fibrin, and infective agents, as described herein. For example, targeted microbubbles can be produced that localize to specific targets expressed in the patient. Desired targets are generally based on, but not limited to, the molecular signature of various pathologies, organs, and/or cells. For example, adhesion molecules (e.g., integrin αᵥβ₃, intercellular adhesion molecule-1 (I-CAM-1), fibrinogen receptor GPIIb/IIIa, and VEGF receptors) are expressed in regions of angiogenesis, inflammation, or thrombus. These molecular signatures can be used to localize contrast agents through the use of targeting molecules, including but not limited to, complementary receptor ligands, targeting ligands, proteins, and fragments thereof. Target cell types include, but are not limited to, endothelial cells, neoplastic cells, and blood cells. The methods described herein optionally use contrast agents targeted to VEGFR2, I-CAM-1, αᵥβ₃ integrin, αᵥ, integrin, fibrinogen receptor GPIIb/IIIa, P-selectin, and mucosal vascular adressin cell adhesion molecule-1. Moreover, using methods described herein and known to those skilled in the art, complementary receptor ligands (e.g., monoclonal antibodies) can be readily produced to target other markers in the patient. For example, antibodies can be produced to bind to tumor marker proteins, organ or cell type specific markers, or infective agent markers. Thus, targeted contrast agents may be targeted, using antibodies, proteins, fragments thereof, aptamers, or other ligands, as described herein, to sites of neoplasia, angiogenesis, thrombus, inflammation, infection, and to diseased or normal organs or tissues, including but not limited to blood, heart, brain, blood vessel, kidney, muscle, lung, and liver. Optionally, the targeted markers are proteins and may be extracellular or transmembrane proteins. The targeted markers, including tumor markers, can be the extracellular domain of a protein. The antibodies or fragments thereof designed to target these marker proteins can bind to any portion of the protein. Optionally, the antibodies can bind to the extracellular portion of a protein, for example, a cellular transmembrane protein. Antibodies, proteins, and fragments thereof can be made that specifically or selectively target a desired target molecule using methods described herein and/or known in the art.

Examples of other contrast agents for use with this specification include, for example, stable free radicals (e.g., stable nitroxides) as well as compounds comprising transition, lanthanide, and actinide elements, which may, if desired, be in the form of a salt or may be covalently or noncovalently bound to complexing agents, including lipophilic derivatives thereof or to proteinaceous macromolecules. Preferable transition, lanthanide, and actinide elements include, for example, Gd(III), Mn(II), Cu(II), Cr(III), Fe(II), Fe(III), Co(II), Er(II), Ni(II), Eu(III), and Dy(III). More preferably, the elements may be Gd(III), Mn(II), Cu(II), Fe(II), Fe(III), Eu(III), and Dy(III), and most preferably, Mn(II) and Gd(III). The foregoing elements may be in the form of a salt, including inorganic salts such as a manganese salt, for example, manganese chloride, manganese carbonate, manganese acetate, and organic salts such as manganese gluconate and manganese hydroxylapatite. Other exemplary salts include salts of iron such as iron sulfides, and ferric salts such as ferric chloride.

The above elements may also be bound, for example, through covalent or noncovalent association, to complexing agents, including lipophilic derivatives thereof or to proteinaceous macromolecules. Preferred complexing agents include, for example, diethylenetriaminepentaacetic acid (DTPA); ethylenediaminetetraacetic acid (EDTA); 1,4,7,10-tetraazacyclododecane-N,N′N″,N′″-tetraacetic acid (DOTA); 1,4,7,10-tetraazacyclododecane-N,N′,N″-triacetic acid (DOTA); 3,6,9-triaza-12-oxa-3,6,9-tricarboxymethylene-10-carboxy-13-phenyltridecan oic acid (B-19036); hydroxybenzylethylenediamine diacetic acid (HBED); N,N′-bis(pyridoxyl-5-phosphate)ethylene diamine; N,N′-diacetate (DPDP); 1,4,7-triazacyclononane-N,N′,N″-triacetic acid (NOTA); 1,4,8,11-tetraazacyclotetradecane-N,N′,N″N′″-tetraacetic acid (TETA); kryptands (macrocyclic complexes); and desferrioxamine. More preferably, the complexing agents are EDTA, DTPA, DOTA, DO3A, and kryptands, most preferably DTPA. Preferable lipophilic complexes include alkylated derivatives of the complexing agents EDTA and DOTA, for example, N,N′-bis-(carboxydecylamidomethyl-N-2,3-dihydroxypropyl)ethylenediamine-N,N′-diacetate (EDTA-DDP); N,N′-bis-(carboxyoctadecylamidomethyl-N-2,3-dihydroxypropyl)ethylenediamine-N,N′-diacetate (EDTA-ODP); and N,N′-Bis(carboxylaurylamidomethyl-N-2,3-dihydroxypropyl)ethylenediamine-N, N′-diacetate (EDTA-LDP), including those described in U.S. Pat. No. 5,312,617, the disclosures of which are hereby incorporated herein by reference in their entirety for all purposes. Preferable proteinaceous macromolecules include, for example, albumin, collagen, polyarginine, polylysine, polyhistidine; γ-globulin and β-globulin, with albumin; with polyarginine, polylysine, and polyhistidine being more preferred. Suitable complexes therefore include: Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine; more preferably, Mn(II)-DTPA or Gd(III)-DTPA.

Nitroxides are paramagnetic contrast agents which increase both T1 and T2 relaxation rates on MRI by virtue of the presence of an unpaired electron in the nitroxide molecule. As known to one of ordinary skill in the art, the paramagnetic effectiveness of a given compound as an MRI contrast agent may be related, at least in part, to the number of unpaired electrons in the paramagnetic nucleus or molecule, and specifically, to the square of the number of unpaired electrons. For example, gadolinium has seven unpaired electrons whereas a nitroxide molecule has one unpaired electron. Thus, gadolinium is generally a much stronger MRI contrast agent than a nitroxide. However, effective correlation time, another important parameter for assessing the effectiveness of contrast agents, confers potential increased relaxivity to the nitroxides. When the tumbling rate is slowed, for example, by attaching the paramagnetic contrast agent to a large molecule, it will tumble more slowly, and thereby more effectively transfer energy to hasten relaxation of the water protons. In gadolinium, however, the electron spin relaxation time is rapid and will limit the extent to which slow rotational correlation times can increase relaxivity. For nitroxides, however, the electron spin correlation times are more favorable and tremendous increases in relaxivity may be attained by slowing the rotational correlation time of these molecules. Although not intending to be bound by any particular theory of operation, since the nitroxides may be designed to coat the perimeters of the vesicles, for example, by making alkyl derivatives thereof, and the resulting correlation times can be optimized. Moreover, the resulting contrast medium of the present disclosure may be viewed as a magnetic sphere, a geometric configuration which maximizes relaxivity.

Superparamagnetic contrast agents suitable for use with the nanocarriers described herein include metal oxides and sulfides which experience a magnetic domain, ferro- or ferrimagnetic compounds such as pure iron; and magnetic iron oxide such as magnetite, γ-Fe₂O₃, Fe₃O₄, manganese ferrite, cobalt ferrite, and nickel ferrite. Along with the gaseous precursors described herein, paramagnetic gases can be employed in the present compositions (e.g., oxygen 17 gas [¹⁷O₂], hyperpolarized xenon, neon, or helium). MR whole body imaging may then be employed to rapidly screen the body, for example, for thrombosis, and ultrasound may be applied, if desired, to aid in thrombolysis.

The contrast agents such as the paramagnetic and superparamagnetic contrast agents described above, may be employed as a component within the compositions of embodiments of the present invention. With respect to vesicles, the contrast agents may be entrapped within the internal void thereof, administered as a solution with the vesicles, incorporated with any additional stabilizing materials, or coated onto the surface or membrane of the vesicle. Mixtures of any one or more of the paramagnetic agents and/or superparamagnetic agents in the present compositions may be used. The paramagnetic and superparamagnetic agents may also be co-administered separately, if desired. In addition, the paramagnetic or superparamagnetic agents may be delivered as alkylated or other derivatives incorporated into the compositions, if desired, especially the polymeric walls of the nanocarriers of the present invention. In particular, the nitroxides 2,2,5,5-tetramethyl-1-pyrrolidinyloxy, free radical, and 2,2,6,6-tetramethyl-1-piperidinyloxy, free radical, can form adducts with, for example, polymers and copolypeptides of the embodiments of the invention.

The iron oxides may simply be incorporated into the contrast agents for use with the present teachings using methods and procedures described previously in this specification. A few large particles may have a much greater effect than a larger number of much smaller particles, primarily due to a larger correlation time. If one were to make the iron oxide particles very large, however, increased toxicity may result, and the lungs may be embolized or the complement cascade system activated. Further, the total size of the particle is not as important as the diameter of the particle at its edge or outer surface. The domain of magnetization or susceptibility effect falls off exponentially from the surface of the particle. Generally, in the case of dipolar (i.e., through space) relaxation mechanisms, this exponential fall off exhibits an r⁶ dependence for a paramagnetic dipole-dipole interaction. Interpreted literally, a water molecule that is 4 Å away from a paramagnetic surface will be influenced 64 times less than a water molecule that is 2 Å away from the same paramagnetic surface. The ideal situation in terms of maximizing the contrast effect would be to make the iron oxide particles hollow, flexible, and as large as possible. By coating the inner or outer surfaces of the nanocarriers of the present invention with the contrast agents, even though the individual contrast agents, for example, iron oxide nanoparticles or paramagnetic ions, are relatively small structures, the effectiveness of the contrast agents may be even further enhanced. In so doing, the contrast agents may function as an effectively much larger sphere wherein the effective domain of magnetization is determined by the diameter of the vesicle and is maximal at the surface of the vesicle. These agents afford the advantage of flexibility, namely, compliance. While rigid vesicles might lodge in the lungs or other organs and cause toxic reactions, these flexible vesicles slide through the capillaries much more easily.

In contrast to the flexible compositions described above, it may be desirable, in certain circumstances, to formulate compositions from substantially impermeable polymeric materials, including, for example, polymethyl methacrylate. This would generally result in the formation of compositions which may be substantially impermeable and relatively inelastic and brittle. In embodiments involving diagnostic imaging, for example, ultrasound, contrast media which comprise such brittle compositions would generally not provide the desirable reflectivity that the flexible compositions may provide. However, by increasing the power output on ultrasound, the brittle compositions (e.g., microspheres) may be made to rupture, thereby causing acoustic emissions, leading to nanocarrier disassociation, therapeutic release, and detection of said acoustic emissions (e.g., by a spectral analyzer).

### Therapeutics

The therapeutic to be delivered by embodiments of this specification may be embedded within the wall of a nanocarrier, encapsulated in the vesicle and/or attached to the surface of the nanocarrier. The phrase “attached to” or variations thereof mean that the therapeutic is linked in some manner to the inside and/or the outside wall of the nanocarrier such as through a covalent or ionic bond or other means of chemical or electrochemical linkage or interaction. The phrase “encapsulated in,” or a variation thereof means that the therapeutic is located in the internal nanocarrier void. The delivery vesicles of the present teachings may also be designed so that there is a symmetric or an asymmetric distribution of the drug, both inside and outside of the stabilizing material and/or nanocarrier. Ultrasonically sensitive materials are especially preferred for use as therapeutics with the present specification.

Any of a variety of therapeutic agents, including those described herein, may be encapsulated in, attached to, and/or embedded in said nanocarriers. If desired, more than one therapeutic may be applied using the vesicles. For example, a single vesicle may contain more than one therapeutic or nanocarriers containing different bioactive agents may be co-administered. In an optimal embodiment, compositions of this disclosure comprise a therapeutic and a targeting moiety. By way of example, a monoclonal antibody capable of binding to a melanoma antigen and an oligonucleotide encoding at least a portion of EL-2 may be administered at the same time. The phrase “at least a portion of” means that, for example, the entire gene need not be represented by the oligonucleotide, so long as the portion of the gene represented provides an effective block to gene expression.

Some of the preferred therapeutic macromolecules for delivery to the patient by embodiments of the present teachings, either attached to or encapsulated within, include genetic material such as nucleic acids, RNA and DNA of either natural or synthetic origin, recombinant RNA and DNA, antisense RNA, microRNAs (miRNAs), shorthairpin RNAs (shRNAs), RNA interference (RNAi), and small interfering RNA (siRNA), including other small RNA-based therapeutics. Other types of genetic material that may be delivered by the nanocarriers described herein include, for example, genes carried on expression vectors such as plasmids, phagemids, cosmids, yeast artificial chromosomes (YACs), defective or “helper” viruses, viral subcomponents, viral proteins or peptides, either alone or in combination with other agents including the therapeutics described herein; and antigene nucleic acids, both single- and double-stranded RNA and DNA, and analogs thereof such as phosphorothioate and phosphorodithioate oligodeoxynucleotides. Additional genetic material that may be delivered to the patient by the present teachings include partially and fully single-stranded and double-stranded nucleotide molecules and sequences, chimeric nucleotides, hybrids, duplexes, heteroduplexes, and any ribonucleotide, deoxyribonucleotide, or chimeric counterpart thereof, and/or corresponding complementary sequence, promoter or primer-annealing sequence needed to amplify, transcribe, or replicate all or part of a biological molecule or sequence. Additionally, the genetic material may be combined with, for example, proteins, polymers, and/or other components including a variety of therapeutics. Other examples of genetic material that may be applied using the nanocarriers of this disclosure include, for example, DNA encoding at least a portion of LFA-3, DNA encoding at least a portion of an HLA gene, DNA encoding at least a portion of dystrophin, DNA encoding at least a portion of CFTR, DNA encoding at least a portion of IL-2, DNA encoding at least a portion of TNF, and an antisense oligonucleotide capable of binding the DNA encoding at least a portion of ras.

In addition, preferred therapeutics include peptides, polypeptides, and proteins such as adrenocorticotropic hormone, angiostatin, Angiotensin Converting Enzyme (ACE) inhibitors (e.g., captopril, enalapril, and lisinopril), bradykinins, calcitonins, cholecystokinins, and collagenases; enzymes such as alkaline phosphatase and cyclooxygenases colony stimulating factors, corticotropin release factor, dopamine, elastins, epidermal growth factors, erythropoietin, transforming growth factors, fibroblast growth factors, glucagon, glutathione, granulocyte colony stimulating factors, granulocyte-macrophage colony stimulating factors, human chorionic gonadotropin, IgA, IgG, IgM, inhibitors of bradykinins, insulin, integrins, interferons (e.g., interferon α, interferon β, and interferon γ); ligands for Effector Cell Protease Receptors, thrombin, manganese super oxide dismutase, metalloprotein kinase ligands, oncostatin M, interleukins (e.g., interleukin 1, interleukin 2, interleukin 3, interleukin 4, interleukin 5, interleukin 6, interleukin 7, interleukin 8, interleukin 9, interleukin 10, interleukin 11, and interleukin 12), opiate peptides (e.g., enkephalines and endorphins); and oxytocin, pepsins, platelet-derived growth factors, lymphotoxin, promoters of bradykinins, Protein Kinase C, streptokinase, substance P (i.e., a pain moderation peptide), tissue plasminogen activator, tumor necrosis factors, nerve growth factors, urokinase, vascular endothelial cell growth factors, and vasopressin.

Other preferred therapeutics such as for the treatment of ophthalmologic diseases and prostate cancer, for use with the nanocarriers described herein, include 15-deoxy spergualin, 17-α-acyl steroids, 3-(Bicyclyl methylene) oxindole, 3α-, 5α-tetrahydrocortisol, 5α-reductase inhibitor, adaprolol enantiomers, aldose reductase inhibitors (e.g., sorbinil and tolrestat), aminoguanidine, antiestrogenics (e.g., 24-(1,2-diphenyl-1-butenyl)phenoxy)-N,N-dimethylethanamine) apraclonidine hydrochloride, aurintricarboxylic acid, azaandrosterone, bendazac, benzoylcarbinol salts, betaxolol, bifemelane hydrochloride, bioerodible poly(ortho ester), cetrorelix acetate, cidofovir, vitamin E, dipifevrin, dipyridamole+aspirin, dorzolamide, epalrestat, etofibrate, etoposide, filgastrim, foscarnet, fumagillin, ganciclovir, granulocyte macrophage colony stimulating factor (GM-CSF), haloperidol, imidazo pyridine, latanoprost, lecosim, levobunolol, N-4 sulphanol benzyl-imidazole, N-acyl-5-hydroxytryptamine, nipradilol, nitric oxide synthase inhibitors, pilocarpine, ponalrestat, prostanoic acid, S-(1,3 hydroxyl-2-phosphonylmethoxypropyl) cytosine, somatuline, sorvudine, ticlopidine, timolol, Trolox™, vaninolol, vascular endothelial growth factor, and α-interferon.

Additional therapeutics suitable for delivery to the patient, either attached or encapsulated within embodiments of the invention, include anti-allergic agents such as amelexanox; Anti-anginals such as diltiazem, erythrityl tetranitrate, isosorbide dinitrate, nifedipine, nitroglycerin (glyceryl trinitrate), pentaerythritol tetranitrate, and verapamil; antibiotics such as amoxicillin, ampicillin, bacampicillin, carbenicillin, cefaclor, cefadroxil, cephalexin, cephradine, chloramphenicol, clindamycin, cyclacillin, dapsone, dicloxacillin, erythromycin, hetacillin, lincomycin, methicillin, nafcillin, neomycin, oxacillin, penicillin G, penicillin V, picloxacillin, rifampin, tetracycline, ticarcillin, and vancomycin hydrochloride; anti-coagulants such as phenprocoumon, and heparin; anti-fungal agents such as polyene antibiotics like flipin, natamycine, and rimocidin; imidazoles such as clotrimazole, ketoconazole, and micronazole; triazoles such as fluconazole, itraconazole, and ravuconazole; allylamines such as amorolfine, butenafine, naftifine, and terbinafine; echinocandins such as caspofungin, micafungin, and ulafungin, and others such as amphotericin B, flucytosine, griseofulvin, miconazole, nystatin, and ricin; anti-inflammatories such as aspirin difimisal, ibuprofen, indomethacin, meclofenamate, mefenamic acid, naproxen, oxyphenbutazone, phenylbutazone, piroxicam, salicylates, sulindac, and tolmetin; anti-neoplastic agents such as adriamycin, aminoglutethimide, amsacrine (m-AMSA), ansamitocin, arabinosyl adenine, arabinosyl, asparaginase (L-asparaginase), Erwina asparaginase, bisitnidazoacridones, bleomycin sulfate, bleomycin, bleomycin, busulfan, carzelesin, chlorambucil, cytosine arabinoside, dactinomycin (actinomycin D), daunorubicin hydrochloride, doxorubicin hydrochloride, estramustine phosphate sodium, etoposide (VP-16), flutamide, interferon α-2a, interferon α-2b, leuprolide acetate mercaptopolylysine, leuprolide acetate, megestrol acetate, melphalan (e.g., L-sarolysin [L-PAM, also known as Alkeran] and phenylalanine mustard [PAM]), mercaptopurine, methotrexate, methotrexate, mitomycin, mitomycin, mitotane, platinum compounds (e.g., spiroplatin, cisplatin, and carboplatin), plicamycin (mithramycin), procarbazine hydrochloride, tamoxifen citrate, taxol, teniposide (VM-26), testolactone, trilostane, vinblastine sulfate (VLB), vincristine sulfate, and vincristine; anti-protozoans such as chloroquine, hydroxychloroquine, metronidazole, quinine, and meglumine antimonate; anti-rheumatics such as penicillamine; and anti-virals such as abacavir, acyclovir, amantadine, didanosine, emtricitabine, enfuvirtide, entecavir, ganciclovir, gardasil, lamivudine, nevirapine, nelfinavir, oseltamivir, ribavirin, rimantadine, ritonavir, stavudine, valaciclovir, vidarabine, zalcitabine, and zidovudine.

Other therapeutics suitable for delivery to the patient, either attached or encapsulated within the nanocarriers described herein, include biological response modifiers such as muramyldipeptide, muramyltripeptide, prostaglandins, microbial cell wall components, lymphokines (e.g., bacterial endotoxin such as lipopoly saccharide, macrophage activation factor, etc.), and bacterial polypeptides such as bacitracin, colistin, and polymixin B; blood products such as parenteral iron, hemin, hematoporphyrins, and their derivatives; cardiac glycosides such as deslanoside, digitoxin, digoxin, digitalin, and digitalis; and circulatory drugs such as propranolol. DNA encoding certain proteins may be used in the treatment of many different types of diseases. For example, adenosine deaminase may be provided to treat ADA deficiency; tumor necrosis factor and/or interleukin-2 may be provided to treat advanced cancers; HDL receptors may be provided to treat liver disease; thymidine kinase may be provided to treat ovarian cancer, brain tumors, or HIV infection; HLA-B7 may be provided to treat malignant melanoma; interleukin-2 may be provided to treat neuroblastoma, malignant melanoma, or kidney cancer; interleukin-4 may be provided to treat cancer; HIV env may be provided to treat HIV infection; antisense ras/p53 may be provided to treat lung cancer; and Factor VIII may be provided to treat Hemophilia B, dyes are included within the definition of a “therapeutic.” Dyes may be useful for identifying the location of a vesicle within the patient's body or particular region of the patient's body. Following administration of the vesicle compositions, and locating, with energy such compositions within a region of the patient's body to be treated, the dye may be released from the composition and visualized by energy. Dyes useful in the present teachings include fluorescent dyes and colorimetric dyes such as 3HCl, 5-carboxyfluorescein diacetate, 4-chloro-1-naphthol, 7-amino-actinomycin D, 9-azidoacridine, acridine orange, allophycocyanin, amino methylcoumarin, benzoxanthene-yellow, bisbenzidide H 33258 fluorochrome, BODIPY FL, BODIPY TMR, BODIPY-TR, bromocresol blue, bromophenol blue, carbosy-SNARF, Cascade blue, chromomycin-A3, dansyl+R—NH₂, DAPI, DTAF, DTNB, ethidium bromide, fluorescein, fluorescein-5-maleimide diacetate, FM1-43, fura-2, Indo-1, lucifer yellow, methylene blue, mithramycin A, NBD, oregon green, propidium iodide, rhodamine 123, rhodamine red-X, R-Phycoerythrin, SBFI, SIST, sudan black, tetramethyl purpurate, tetramethylbenzidine, tetramethylrhodamine, texas red, thiazolyl blue, TRITC, YOYO-1, and the like. Fluorescein may be fluorescein isothiocyanate. The fluorescein isothiocyanate includes inter alia, fluorescein isothiocyanate albumin, fluorescein isothiocyanate antibody conjugates, fluorescein isothiocyanate α-bungarotoxin, fluorescein isothiocyanate-casein, fluorescein isothiocyanate-dextrans, fluorescein isothiocyanate-insulin, fluorescein isothiocyanate-lectins, fluorescein isothiocyanate-peroxidase, and fluorescein isothiocyanate-protein A.

Additional therapeutics suitable for delivery to the patient, either attached or encapsulated within the nanocarriers of this disclosure, include general anesthetics such as droperidol, etomidate, fentanyl citrate with droperidol, ketamine hydrochloride, methohexital sodium, and thiopental sodium, and radioactive particles or ions such as strontium, iodide rhenium, technetium, cobalt, and yttrium. In certain preferred embodiments, the bioactive agent is a monoclonal antibody or a monoclonal antibody fragment such as a monoclonal antibody capable of binding to melanoma antigen; hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunsolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, progesterone, testosterone, and adrenocorticotropic hormone; local anesthetics such as bupivacaine hydrochloride, chloroprocaine hydrochloride, etidocaine hydrochloride, lidocaine hydrochloride, mepivacaine hydrochloride, procaine hydrochloride, and tetracaine hydrochloride; metabolic potentiators such as glutathione; antituberculars such as para-aminosalicylic acid, isoniazid, capreomycin sulfate cycloserine, ethambutol hydrochloride ethionanide, pyrazinamide, rifampin, and streptomycin sulfate; narcotics such as paregoric, and opiates such as codeine, heroin, methadone, morphine, and opium; neuromuscular blockers such as atracurium besylate, gallamine triethiodide, hexafluorenium bromide, metocurine iodide, pancuronium bromide, succinylcholine chloride (suxamethonium chloride), tubocurarine chloride, and vecuronium bromide; sedatives (i.e., hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam, and triazolam; subunits of bacteria (e.g., Mycobacteria and Corynebacteria), the synthetic dipeptide N-acetyl-muramyl-L-alanyl-D-isoglutamine, and the like; and vitamins such as cyanocobalamin neinoic acid, retinoids and derivatives such as retinol palmitate, α-tocopherol, naphthoquinone, cholecalciferol, folic acid, and tetrahydrofolate.

The aforementioned therapeutics and their precursors and modifications are only representative of the plethora of compounds suitable for delivery to the patient by embodiments of this disclosure. A large number of molecular variables can be altered with nearly all of these illustrative embodiments; thus, a wide variety of drugs, genes, and other compounds and structures have the capability of being delivered alone or in combination with other materials by embodiments of the present invention. Optimally, said therapeutics will be specifically designed and engineered for acoustically mediated drug and gene delivery.

### Ultrasound and Drug Delivery

In 1954, the successful treatment of digital polyarthritis using hydrocortisone in combination with ultrasound was reported (Fellinger et al., 1954). Half a century later, this technique, sometimes called sonophoresis or phonophoresis, has emerged as a powerful tool for facilitating transdermal drug delivery, allowing needle-free drug administration. Skin prohibits the transport of macromolecular drugs such as proteins; therefore, needles are still required as the primary mode of administration of therapeutic macromolecules.

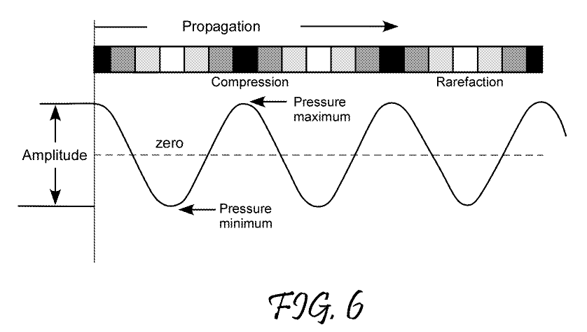

By definition, ultrasound is sound having a frequency greater than 20,000 cycles per second (i.e., sound above the audible range). Acoustic (i.e., sound) waves are merely organized vibrations of the molecules or atoms of a medium capable of supporting the propagation of said wave. Usually, the vibrations are organized in a sinusoidal fashion which readily reflects areas of compression and rarefaction (FIG. 6). Such changes are frequently depicted or described as a sine wave with the peak of the “hill” representing the pressure maximum and the nadir of the “valley” representing the pressure minimum. The areas of compression and refraction are due to periodic pressure being applied to the surface of the medium which is, in the most preferred embodiments of the present invention, human tissue (FIG. 6).

Simplistically, ultrasound is generated by a transducer which converts electrical energy to acoustical energy, or vice versa. Nearly all transducers use piezoelectric materials, and can be either a natural crystalline solid (e.g., quartz) or a manufactured ceramic (e.g., barium titanate or zirconate titanate). To produce ultrasound, a suitable voltage is applied to the transducer. When the frequency of the input voltage reaches the resonance frequency of the piezoelectric material, the piezoelectric material responds by undergoing vibrations. Thus, a piezoelectric crystal can produce a pulse of mechanical energy (i.e., pressure pulse; FIG. 6) by electrically exciting the crystal, functioning as a transmitter; and as a transducer, producing a pulse of electrical energy by mechanically exciting the crystal and thus functioning as a receiver. Either single- or multiple-(phased) transducers may be utilized in ultrasonic instrumentation.

In 1956, Burov suggested that high-intensity ultrasound (HIFU) could be used for the treatment of cancer; in the years following, several studies looked at the effect of ultrasound on tissues (Taylor et al., 1969). Embodiments of the present invention highlight a new clinical and laboratory use of HIFU, mediating intracellular drug delivery in vivo. Because the goal of this HIFU application is usually not cell death at the treatment focus, for the purposes of the present disclosure, increases in heat at the treatment area must be carefully monitored and controlled.

While not wishing to be bound by any particular theory, HIFU's mode of action on living tissue is probably through two predominant mechanisms. The first is by a thermal mechanism (i.e., hyperthermia), the conversion of mechanical energy into heat. Whenever ultrasonic energy is propagated into material (e.g., tissue), the amplitude of the wave decreases with distance. This attenuation is due either to energy absorption or scattering. Absorption is a mechanism where a portion of the wave energy is converted into heat, and scattering is where a portion of the wave changes direction. Because tissue can absorb energy to produce heat, a temperature increase may occur as long as the rate heat produced is greater than the rate heat removed. This thermal mechanism is relatively well understood because an increase in temperature caused by ultrasound can be calculated using mathematical modeling techniques.

Briefly, healthy cellular activity depends on chemical reactions occurring at the proper location, at the proper rate. The rates of these chemical reactions, and thus of enzymatic activity, are temperature dependent. The overall effect of temperature on enzymatic activity is described by the relationship known as the 10° temperature coefficient, or Q₁₀ Rule (Hille, 2001). Many enzymatic reactions have a Q₁₀ near 3 which means that for each 10° C. increase in temperature, enzymatic activity increases by a factor of 3. An immediate consequence of a temperature increase is an escalation in biochemical reaction rates. However, when the temperature becomes sufficiently high (i.e., approximately ≧45° C.), enzymes denature. Subsequently, enzymatic activity decreases and ultimately ceases, which can have a significant impact on cell structure and function. The extent of damage induced by hyperthermia will be dependent on the duration of the exposure as well as on the temperature increase achieved. Detrimental effects in vitro are generally noted at temperatures of 39-43° C., if maintained for a sufficient time period; at higher temperatures (i.e., >44° C.), coagulation of proteins occurs rapidly.

Without wishing to be bound by any particular theory, HIFU's second major mode of action on living tissue, and whose effect is most important for the purposes of embodiments of this specification, is believed to be acoustic cavitation. Cavitation is a complex phenomena; at the time of this writing, it is somewhat unpredictable especially because of instrument limitations. However, for the purposes of embodiments of this disclosure, if it is not controlled, the end result, as with hyperthermia, is also cell necrosis, induced through a combination of mechanical stresses and thermal injury. Ultrasound causes tissues to vibrate, where cellular molecular structure is subjected to alternating periods of compression and rarefaction (FIG. 6). During rarefaction, gas can be drawn out of solution to form bubbles, which can oscillate in size, or collapse (i.e., implode) rapidly, causing mechanical stresses and generating temperatures of 2,000-5,000° K. in the microenvironment surrounding the bubble. Cavitation is dependent, among other things, on acoustic energy pulse length, frequency, and intensity, and is unlikely to occur when using diagnostic ultrasound equipment. At this time, the impact on tissue induced by hyperthermia is both more repeatable and predictable than by cavitation. This undoubtedly is a major factor in making heat the preferred mode of therapeutic action in early clinical applications of HIFU.

Information relevant to attempts to use ultrasound in delivering drugs to specific regions of a patient in vivo using an ultrasonically active gas or gaseous precursor-filled lipid microspheres (i.e., termed “lipospheres”) can be found in, for example, U.S. Pat. Nos. 5,770,222; 5,935,553; 6,071,495; 6,139,819; 6,146,657; 6,403,056; 6,416,740; 6,773,696; 6,998,107; and 7,083,572. These preceding applications are seriously limited, because, for example, multicomponent, non-covalently associated systems are challenging to formulate and stabilize; and difficulties with storage/stability and short shelf-life; unmodified lipospheres activate complement, a basic component of the immune system, and cause pseudo-allergic reactions that can damage heart and liver cells; large-scale manufacturing of lipidic carrying vesicles is still very challenging, even with recent technological advances in sterile techniques and process controls; optimization of the long-term physical stability of liposomal formulations remains a critical task in new product development; and most lipospheres are limited to carrying predominantly hydrophobic drugs, and they have a reduced capacity to deliver higher levels of these therapeutics to the treatment sight.

### Ultrasound Instruments and Parameters

After reviewing the cellular mechanisms that appear to be responsible for macromolecular uptake in insonated cells, instrumentation to measure, monitor, and control the amount and extent of acoustic cavitation induced by acoustic energy transferred to the patient would be the most preferred type of device to use with the present teachings. Unfortunately, as of yet, no such FDA-approved or even experimental device exists. Therefore, existing diagnostic and HIFU instrumentation may be employed in the practice of the present teachings. For example, any of the various types of diagnostic ultrasound imaging devices may be employed, the particular type or model of the device not being critical to the method of the invention. Additionally, devices designed for administering ultrasonic hyperthermia are suitable to be used with said imaging devices in the practice of the invention such devices being described in, for example, U.S. Pat. Nos. 4,620,546, 4,658,828, and 4,586,512, and the disclosures of each of which are hereby incorporated herein by reference in their entirety for all purposes. Recently, a new commercially available system became available—the Sonablate 500® by Misonix—available through International HIFU Central, and described in U.S. patent application Ser. No. 11/177,827, filed on Jul. 8, 2005; the disclosures of which are incorporated by reference in their entity for all purposes. This is a HIFU system designed with enhancements for the treatment of prostatic diseases. However, it is contemplated that the instrument with a suitable transducer will be useful in the practice of embodiments of this disclosure.

Ultrasound devices may be more optimally used with the present teachings, for example, by employing some of the following instrumentation parameters. In general, devices for therapeutic ultrasound should employ from approximately 10% to approximately 100% pulse durations, depending on the area of tissue to be treated. A region of the patient which is generally characterized by larger amounts of muscle mass, (e.g., the back and thighs) as well as highly vascularized tissues (e.g., heart tissue) may require a larger duty factor, for example, up to approximately 100% (i.e., continuous). In therapeutic ultrasound, continuous wave ultrasound is used to deliver higher energy levels. For rupturing the nanocarriers of the present invention, continuous wave ultrasound may, in some circumstances, be preferred, although the sound energy is usually pulsed, especially in order to optimize acoustic cavitation and minimize temperature increases at the target site. If pulsed sound energy is used, the sound will generally be pulsed in echo train lengths of approximately 8 pulses to approximately 20 or more pulses at a time.

In addition to the pulsed method, continuous wave ultrasound (e.g., Power Doppler) may be applied. This may be particularly useful where rigid vesicles (e.g., nanocarriers that are extensively cross-linked) are employed. In this case, the relatively higher energy of the Power Doppler may be made to resonate ultrasound contrast agents co-administered with the nanocarriers of the present teachings, thereby promoting their rupture. Indeed, as described herein, this can create acoustic emissions which may be in the subharmonic or ultraharmonic range or, in some cases, in the same frequency as the applied ultrasound. Generally, the levels of energy from diagnostic ultrasound should be insufficient to promote the rupture of vesicles and to facilitate release and cellular uptake of any bioactive agents. As noted previously, diagnostic ultrasound may involve the application of one or more pulses of sound. Pauses between pulses permit the reflected sonic signals to be received and analyzed. Thus, the limited number of pulses used in diagnostic ultrasound limits the effective energy which is delivered to the tissue under treatment.

Higher-energy ultrasound (i.e., ultrasound which is generated by therapeutic ultrasound equipment) is usually capable of causing rupture of embodiments of the present invention. The frequency of the sound used may vary from approximately 0.025 MHz to approximately 10 MHz. In general, frequency for therapeutic ultrasound preferably ranges between approximately 0.75 MHz and approximately 3 MHz, with from approximately 1 MHz to approximately 2 MHz being more preferred. In addition, energy levels may vary from approximately 0.5 Watt (W) per square centimeter (cm²) to approximately 5.0 W/cm², with energy levels from approximately 0.5 W/cm² to approximately 2.5 W/cm² being preferred. Energy levels for therapeutic ultrasound causing hyperthermia are generally from approximately 5 W/cm² to approximately 50 W/cm². For small vesicles (e.g., vesicles having a diameter of less than approximately 0.5 μm) higher frequencies of sound are generally preferred because smaller vesicles are capable of absorbing sonic energy more effectively at higher frequencies of sound. When very high frequencies are used (i.e., greater than approximately 10 MHz), the sonic energy will generally penetrate fluids and tissues to a limited depth only. Thus, external application of the sonic energy may be suitable for skin and other superficial tissues. However, it is generally necessary for deep structures to focus the ultrasonic energy so that it is preferentially directed within a focal zone. Alternatively, the ultrasonic energy may be applied via interstitial probes, intravascular ultrasound catheters, or endoluminal catheters.

For therapeutic drug delivery, after the compositions described herein have been administered to, or have otherwise reached the target region (e.g., via delivery with targeting ligand), the rupturing of the therapeutic containing nanocarriers of this specification is carried out by applying ultrasound of a certain total exposure time, duty factor, pulse length, and peak incident pressure and frequency, to the region of the patient where therapy is desired. Specifically, when ultrasound is applied at a frequency corresponding to the peak resonant frequency of, for example, gaseous ultrasound contrast agents (i.e., microbubbles) co-administered with said therapeutic-containing nanocarriers, the vesicles should rupture and release their contents at the target area in part because of shockwaves produced by said cavitating microbubbles, as described herein. The peak resonant frequency can be determined either in vivo or in vitro, but preferably in vivo, by exposing the compositions to ultrasound, receiving the reflected resonant frequency signals and analyzing the spectrum of signals received to determine the peak, using conventional means. The peak, as so determined, corresponds to the peak resonant frequency, or second harmonic, as it is sometimes termed.

The therapeutic-containing nanocarriers should also rupture when, for example, co-administered ultrasound contrast agents are exposed to non-peak resonant frequency ultrasound in combination with a higher intensity (i.e., wattage) and duration (i.e., time). This higher energy, however, results in greatly increased heating and tissue damage. By adjusting the frequency of the energy to match the peak resonant frequency of, for example, the gaseous contrast agents co-administered with said therapeutic-containing nanocarriers, the efficiency of therapeutic rupture and release should be improved, and appreciable tissue heating should not occur (i.e., no increase in temperature above approximately 2° C.), because less overall energy is ultimately required for the release of said therapeutic.

A therapeutic ultrasound device may be used with the present teachings which employs two frequencies of ultrasound. The first frequency may be x, and the second frequency may be, for example, 2x. It is contemplated such a device might be designed such that the focal zones of the first and second frequencies converge to a single focal zone at the target area. The focal zone of the device may then be directed to the targeted compositions, for example, targeted vesicle compositions, within the targeted tissue. This ultrasound device may provide second harmonic therapy with simultaneous application of the x and 2x frequencies of ultrasonic energy. In the case of ultrasound involving vesicles, it is contemplated that this second harmonic therapy may provide improved rupturing of vesicles as compared to ultrasonic energy involving a single frequency. Lower energy may also be used with this dual frequency therapeutic ultrasound device, resulting in less sonolysis and cytotoxicity in the target area.

Preferably, the ultrasound device used in the practice of the present invention employs a resonant frequency (RF) spectral analyzer. The transducer probes may be applied externally or may be implanted. Ultrasound is generally initiated at lower intensity and duration, and then intensity, time, and/or resonant frequency is increased. Although application of these various principles will be readily apparent to one skilled in the art, in view of the present disclosure, by way of general guidance, the resonant frequency will generally be in the range of approximately 1 MHz to approximately 10 MHz. Using the 7.5 MHz curved array transducer as an example, adjusting the power delivered to the transducer to maximum and adjusting the focal zone within the target tissue, the spatial peak temporal average (SPTA) power will then be a maximum of approximately 5.31 mW/cm² in water. This power should cause some release of therapeutic agents from nanocarriers in close proximity to gas-filled microbubbles, with much greater release being accomplished by using a higher power.

As described in detail herein, the present teachings function most optimally primarily because of the phenomena of inertial cavitation in rupturing the nanocarriers of this disclosure, releasing and/or activating the bioactive agents within said vesicles. Thus, lower frequency energies may be used, as cavitation occurs more effectively at lower frequencies. Using a 0.757 MHz transducer driven with higher voltages (i.e., as high as 300 volts), cavitation of solutions of gas-filled ultrasound contrast agents will occur at thresholds of approximately 5.2 atmospheres. The ranges of energies transmitted to tissues from diagnostic ultrasound on commonly used instruments is known to one skilled in the art and described, for example, by Carson et al. (1978), the disclosure of which is hereby incorporated herein by reference in its entirety for all purposes. In general, these ranges of energies employed in pulse repetition are useful for diagnosis and monitoring compositions, but should be insufficient to rupture most of the nanocarriers of the present invention.

Either fixed frequency or modulated frequency ultrasound may be used in practicing the present invention. Fixed frequency is defined wherein the frequency of the sound wave is constant over time. A modulated frequency is one in which the wave frequency changes over time, for example, from high to low (i.e., PRICH) or from low to high (i.e., CHIRP). For example, a PRICH pulse with an initial frequency of 2.5 MHz of sonic energy is swept to 50 kHz with increasing power from 1 watt to 5 watts. Focused, frequency-modulated, high-energy ultrasound may increase the rate of local gaseous expansion within ultrasound contrast agents co-administered with the compositions described herein, thereby rupturing said nanocarriers to provide local delivery of therapeutics.

### Best Mode of Practice

The following is a description of a single preferred method for practicing embodiments of the present invention, and should in no way be considered limiting. As this embodiment is described with reference to the aforementioned drawings and definitions, various modifications or adaptations of the methods, materials, and specific techniques described herein may become apparent to those skilled in the art. All such modifications, adaptations, or variations that rely on the teachings of the present invention, and through which these teachings have advanced the art, are considered to be within the spirit and scope of the present invention.

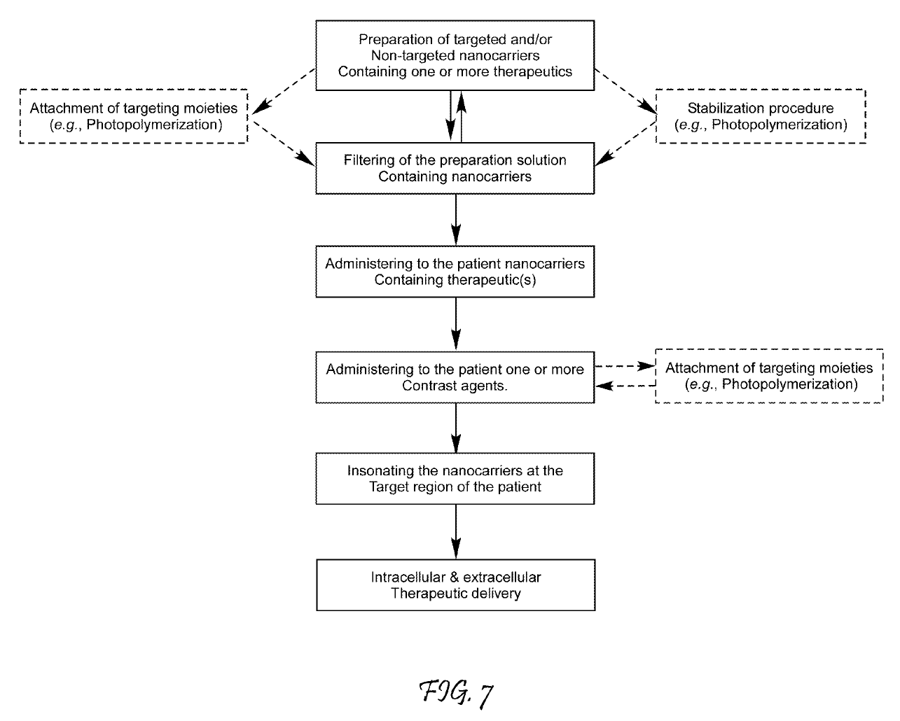

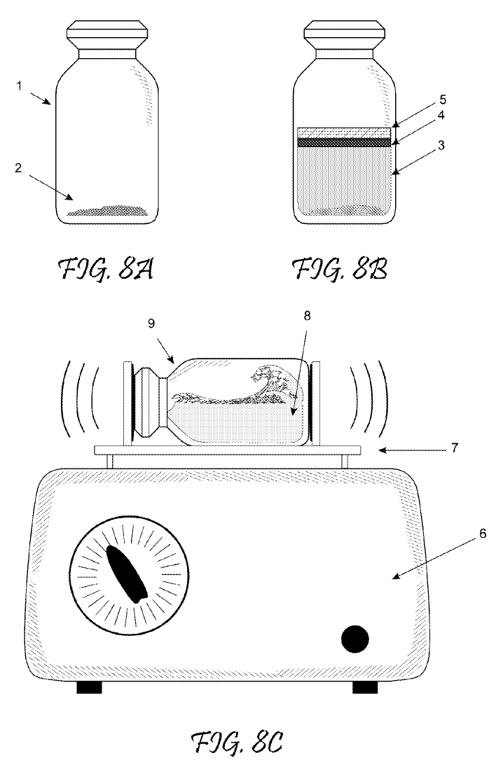

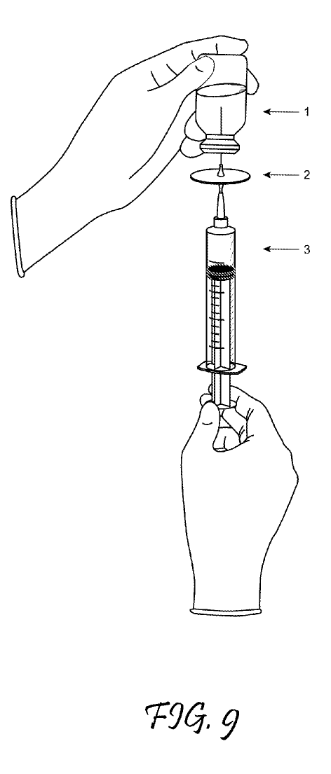

A preferred method for practicing the present invention in a clinical or laboratory environment (FIG. 7), using nanocarriers comprised substantially of supramolecular complexes, involves the following:
1. Preparation of targeted and/or non-targeted nanocarriers containing one or more therapeutics, wherein said therapeutics may be the same as or different from one another (FIGS. 8A-C)
2. Filtering of the preparation solution containing said therapeutic-containing nanocarriers (FIG. 9).
3. Administering to the patient a quantity of said targeted and/or non-targeted nanocarriers.
4. Administering to the patient a quantity of one or more targeted and/or non-targeted contrast agents, wherein said contrast agents may be the same as or different from one another.
5. Insonating the nanocarriers at the target region of the patient with therapeutic ultrasonic waves at a frequency and energy level to cause rupture of said therapeutic containing nanocarriers. However, the energy level must not be so great as to cause significant sonolysis and cytotoxicity at the target site.
6. Possibly receiving ultrasonic and other emissions simultaneously from said contrast and/or other agents, generating an image of said region from the received ultrasonic emissions and other data.
7. Possibly repeating steps 1 through 6, independently or in various combinations, one or more times (FIG. 7).

As one skilled in the art will immediately recognize, once armed with the present disclosure, different nanocarrier preparation methods may be used, as well as widely varying amounts of nanocarriers and contrast agents may be employed in the practice of this preferred embodiment of this specification. As used herein, the phrases “a quantity of said targeted and/or non-targeted nanocarriers” and “quantity of one or more targeted and/or non-targeted contrast agents” are intended to encompass all such amounts.

Nanocarriers may be administered to the patient in a variety of forms adapted to the chosen route of administration, namely, parenterally, orally, or intraperitoneally. Parenteral administration, which is the most preferred method, includes administration by the following routes: intravenous, intramuscular, interstitially, intraarterial, subcutaneous, intraocular, and intrasynovial; transepithelial, including transdermal; pulmonary via inhalation, ophthalmic, sublingual, and buccal; and topically including ophthalmic, dermal, ocular, rectal, and nasal inhalation via insufflation. Intravenous administration is preferred among the routes of parenteral administration.

The useful dosage to be administered and the mode of administration will vary depending on the age, weight, and type of patient to be treated, and the particular therapeutic application intended. Typically, dosage is initiated at lower levels and increased until the desired therapeutic effect is achieved. The patient may be any type of animal, but is preferably a vertebrate, more preferably a mammal, and most preferably human. By “region of a patient,” “target”, or “target site,” it is meant the whole patient, or a particular area or portion of the patient.

The method of the present teachings can also be carried out in vitro (i.e., in cell culture applications, where the nanocarriers and contrast agents may be added to the cells in cultures and then incubated). Therapeutic ultrasonic waves can then be applied to the culture media containing the cells and nanocarriers.

The aforementioned is a description of a single preferred method for practicing the methods of the present disclosure. A plethora of variables can be altered with this clinical or laboratory treatment protocol; therefore, a wide variety of techniques, materials, and other properties are available for the preparation of targeted and non-targeted nanocarriers and contrast agents for acoustically mediated drug delivery, as well as administration and activation procedures of said components.

### EXAMPLES

A more complete understanding of embodiments of this specification will be obtained from the following Examples, all of which are prospective (i.e., prophetic). These examples are intended to be exemplary only and non-limiting to embodiments of the present invention. The chemicals, materials, reagents, glassware, equipment, and instrumentation necessary for the synthesis, purification, and characterization, and evaluation of embodiments of this disclosure are readily known and available to those skilled in the art. The aforementioned biodegradable polymer publications listed in paragraphs (0149] and [0150], and now apart of this specification, describe in detail unique methods and compositions to generate biodegradable polymers and related materials, each with varied properties. The synthesis of PEG-b-oligo(DTO-SA)-b-PEG and PEG-b-oligo(DTO carbonate)-b-PEG self-assembling nanocarriers will be presented.

### Example 1

### Prospective Example: Synthesis of Polyethylene Glycol-Block-Oligo [Desaminotyrosyl-Tyrosine Octyl Ester Suberate]-Block-Polyethylene Glycol [Peg-b-Oligo (DTO-SA)-b-PEG]

This prospective example illustrates the synthesis of polyethylene glycol-block-oligo(desaminotyrosyl-tyrosine octyl ester suberate)-block-polyethylene glycol (PEG-b-oligo [DTO-SA]-b-PEG). In a 100 ml round-bottomed flask, 2.21 gm (0.005 mole) of DTO, 0.96 gm (0.0055 mole) of suberic acid, 0.59 gm (0.002 mole) of 4-dimethylaminopyridinium-p-toluene sulfate, and 25 ml of methylene chloride (DCM) are combined at 293° K. While stirring continuously at room temperature, 1.8 gm (0.014 mole) of diisopropylcarbodiimide (DIPC) is added to the suspension. At regular intervals, aliquots are withdrawn from the mixture for gel permeation chromatography (GPC). When the number average molecular weight (Mₙ) and the weight average molecular weight (M) of the reaction mixture reaches approximately 7,000 and 15,000 respectively (relative to polystyrene standards), 1.1 gm of poly(ethylene glycol) monomethyl ether and 0.4 gm of DIPC are added. Following 2 hours of additional stirring, the reaction mixture is filtered through a sintered glass funnel, and the filtrate evaporated to 10 ml, and then precipitated with 2-propanol. This precipitate is dried, dissolved in 10 ml of DCM, and reprecipitated with 50 ml of methanol, and dried under a vacuum at room temperature.

### Example 2

### Prospective Example: Synthesis of Polyethylene Glycol-Block-Oligo [Desaminotyrosyl-Tyrosine Octyl Carbonate]-Block-Polyethylene Glycol (PEG-b-Oligo [DTO-Carbonate]-b-PEG

This prospective example illustrates the synthesis of polyethylene glycol-block-oligo (desaminotyrosyl-tyro sine octyl carbonate)-block-polyethylene glycol (PEG-b-oligo [DTO-carbonate]-b-PEG). A 0.5 liter 3-necked flask is equipped with a mechanical stirrer and pump and purged with N₂ for 15 minutes. PEG is added to the flask followed by triphosgene (TP; solid) and DCM (HPLC grade). The mixture is stirred to obtain a clear solution (10-15 minutes). At this point, the reaction mixture contains “activated PEG”—PEG-chloroformate—and an excess of unreacted TP. DTO and DCM are placed in a bottle with a screw cap, and the TP solution is added to the reaction mixture using an fluid metering (FMI) pump (over 1 hour). After the addition is complete, 10 ml of D CM is added to the bottle and the reaction mixture over 10 minutes using the pump. After addition is complete, a 150 ml aliquot is withdrawn, evaporated to dryness with air and diluted with 1 ml of THF, filtered, and the filtrate analyzed by GPC to determine the molecular weight distribution of the synthesized compounds. GPC chromatogram should reveal a mixture of products and reacted monomers.

The entire reaction mixture is concentrated by evaporation to a thick syrup (˜10 ml total), which is then precipitated with 60 ml of 2-propanol (drop-wise addition) and allowed to settle. The product obtained should be a yellow thick oil, and the solvents are then decanted off. The precipitate is dried (under N₂) for 20 minutes, redissolved in 10 ml of DCM and precipitated with 60 ml of 2-propanol. This process is repeated 2 more times with (1) 50 ml of methanol:IPA=1:1, and (2) 50 ml methanol. The product (a thick gum) is dried under a stream of nitrogen followed by vacuum drying.

The polycarbonate middle blocks of the present disclosure can be prepared by the conventional methods for polymerizing diphenols into the same, as described by U.S. Pat. No. 5,099,060 (Kohn et al., 1992), the disclosures of which are incorporated herein by reference in their entirety for all purposes. These methods involve the reaction of amino acid-derived diphenol compounds, including those described in U.S. Pat. No. 4,980,449 (Kohn et al., 1990), the disclosures of which are incorporated herein by reference in their entirety for all purposes, with phosgene or phosgene precursors (e.g., diphosgene or triphosgene) in the presence of a catalyst. Suitable processes, associated catalysts, and solvents are known in the art and taught in Chemistry and Physics of Polycarbonates (Shnell, 1964), the disclosures of which are incorporated herein by reference in their entirety for all purposes.

Polymerizing oligomers having pendant free carboxylic acid groups from diphenols, with pendant free carboxylic acid groups, without cross-reaction of the free carboxylic acid groups with the co-monomer, is now possible. Accordingly, homopolymers or copolymers of benzyl ester diphenyl monomers such as DTBn may be converted to corresponding free carboxylic acid homopolymers and copolymers through the selective removal of the benzyl groups by the palladium catalyzed hydrogenolysis method disclosed by U.S. Pat. No. 6,120,491, the disclosures of which are incorporated by reference herein in their entirety for all purposes. The catalytic hydrogenolysis is necessary because the lability of the oligomer backbone prevents the employment of harsher hydrolysis techniques.

### Characterization of Tyrosine Triblock Copolymers

Biodegradable triblock polymers are characterized (minimally) by nuclear magnetic resonance, gel permeation chromatography, and critical micelle concentration. Brief summaries of these procedures are as follows. TABLE 1 summaries a variety of properties commonly observed for suspensions of several different PEG-b-oligo(DTR-SA)-b-PEG triblock copolymers.

### Example 3

### Prospective Example: ¹H Nuclear Magnetic Resonance [NMR] Spectroscopy Evaluation of Tyrosine Triblock Copolymers

This prospective example illustrates the 1H nuclear magnetic resonance (NMR) spectroscopy evaluation of biodegradable triblock copolymers. ¹H NMR spectra are recorded with a suitable spectrophotometer (i.e., 300 MHz). Spectra are obtained with or without TFAA (trifluoroacetic anhydride, 99+%) as a shifting reagent. With DMSO_{-d6} as the solvent, the central line of DMSO at 2.50 ppm is used as the reference line. With CHCl₃ as the solvent, the CHCl₃ signal at 7.26 ppm is used as the reference line.

### Example 4

### Prospective Example: Gel Permeation Chromatography [GPC] of Tyrosine Triblock Copolymers

The molecular weights of the different polymers and their distribution are most easily determined by GPC. Briefly, chromatography columns containing material such as Plgel 3 (Polymer Laboratories) are

TABLE 1
Prospective data illustrating molecular weight properties of the
PEG-b-oligo(DTR-SA)-b-PEG triblock copolymers, and their
corresponding nanocarrier hydrodynamic diameters.

| | | | | | Triblock | Nanocarrier |
|---|---|---|---|---|---|---|
| Polymers/ | Oligo (DTR-SA) | Oligo (DTR-SA) | Triblock^{a} | Triblock^{b} | Polydispersity^{c} | Hydrodynamic |
| Nanocarriers | Block Mₙ | Block M_{w} | Mₙ | M_{w} | (M_{w}/Mₙ) | Diameter, nm |
| 2K/DTE-SA | 6,400 | 11,300 | 10,800 | 21,000 | 1.94 | 44.0 ± 23.6 |
| 5K/DTE-SA | 6,100 | 11,200 | 15,200 | 25,100 | 1.65 | 67.3 ± 32.2 |
| 2K/DTB-SA | 6,000 | 11,700 | 10,300 | 19,800 | 1.92 | 44.8 ± 18.3 |
| 5K/DTB-SA | 6,100 | 11,800 | 15,400 | 25,300 | 1.64 | 70.8 ± 30.3 |
| 2K/DTBn-SA | 6,300 | 13,600 | 10,800 | 21,500 | 1.99 | 47.2 ± 16.0 |
| 5K/DTBn-SA | 6,400 | 13,300 | 15,700 | 25,000 | 1.59 | 68.0 ± 30.9 |
| 2K/DTO-SA | 6,800 | 13,000 | 11,500 | 20,200 | 1.76 | 47.5 ± 30.9 |
| 5K/DTO-SA | 6,100 | 12,600 | 15,500 | 25,000 | 1.61 | 73.6 ± 31.5 |

^{a}The number average molecular weight is a way of determining the molecular weight of a polymer. Polymer molecules, even ones of the same type, come in different sizes (chain lengths, for linear polymers), so the average molecular weight will depend on the method of averaging. The number average molecular weight is the common, mean, average of the molecular weights of the individual polymers, and is determined by measuring the molecular weight of n polymer molecules, summing the weights, and dividing by n.
^{b}The weight average molecular weight is a way of describing the molecular weight of a polymer. Polymer molecules, even if of the same type, come in different sizes (chain lengths, for linear polymers), so an average of some kind must be calculated. The weight average molecular weight can be determined by light scattering, small angle neutron scattering (SANS), X-ray scattering, and sedimentation velocity.
^{c}The polydispersity index (PDI) is a measure of the distribution of molecular weights in a given polymer sample. The PDI calculated is the weight average molecular weight divided by the number average molecular weight, and indicates the distribution of individual molecular weights in a batch of polymers. The PDI has a value always greater than 1, but as the polymer chains approach uniform chain length, the PDI approaches unity.

used with conventional chromatography systems along with a refractometer. Poly(ethylene glycol)s of defined molecular weights may be used as standards. Dimethylformamide (DMF) containing 10 mM LiCl is used as eluent. The samples are dissolved overnight at a concentration of 5 mg/ml in the eluent, and passed through a 0.45 μm filter prior to analysis.

### Example 5

### Prospective Example: Critical Micelle Concentration (CMC) Determination of Tyrosine Triblock Copolymers

This prospective example illustrates the critical micelle concentration (CMC) determination of tyrosine triblock copolymers are determined by static light scattering (SLS) measurements. Briefly, SLS is performed at 303° K. using a multi-angle light scattering instrument equipped with a GaAs laser (wavelength λ=690 nm). Prior to injection, samples are cleared by filtration through a 0.1 μm Millipore syringe filter. The refractive index increment is obtained at the corresponding wavelength and temperature by using a differential refractometer to measure the refractive index of an assembled nanocarrier's dispersion at different concentrations. Given the unexpectedly high value of the refractive index increment of the nanocarrier solution, SLS measurements at several angles and concentrations are extrapolated in a Zimm plot, and self-assembly properties, including average molecular weight, radius of gyration, and second virial coefficient are calculated. The CMC is calculated using the close association model restrictions.

The triblock copolymers of the present invention form vesicular structures in dilute aqueous solutions in the 5-200 nm range (diameter). Preferred structures have diameters between 50 and 150 nm. For example, poly(ethylene glycol)-block-oligo-(DTO suberate)-block-poly(ethylene glycol), i.e., PEG-b-oligo-(DTO suberate)-b-PEG triblock oligomer, forms vesicular structures in dilute aqueous solution having a diameter of about a 100 nm range. The vesicles are characterized with conventional techniques, i.e., light scattering and transmission electron microscopy.

### Example 6

### Prospective Example: Self-Assembly of Nanocarriers from Tyrosine Triblock Copolymers

This prospective example illustrates the self-assembly of nanocarriers from tyrosine triblock copolymers. Briefly, vesicle self-assembly is performed by dissolving, for example, 10 mg of PEG-oligo(DTO suberate)-PEG in 0.2 gm THF, and adding said mixture dropwise to 4.79 gm of water (18 MΩcm⁻¹) under mild agitation. To achieve uniform particle size, the resulting turbid dispersion is sequentially filtered through 0.45, 0.22, and 0.1 μm size syringe filters. All subsequent characterizations are performed using this final filtered preparation. Trace organic solvent contamination is removed by either gentle nitrogen blow-drying or size exclusion chromatography.

The prior art has established that self-assembly of amphiphilic molecules depends on several correlated properties of the underlying material, i.e., its chemical structure, architecture, and/or molecular weight. However, assuming that the driving force of the self-assembly is mainly governed by hydrophobic interactions, the design of a self-assembling block copolymer inherently depends on its molecular weight, and hydrophobic to hydrophilic balance. The self-assembly of the triblock copolymers in dilute aqueous solution is induced by simple dropwise addition and may be facilitated by sonication, high shear mixing, nanoprecipitation or emulsification methods. Active hydrophobic products are complexed by premixing the triblocks and hydrophobic products in a suitable solvent prior to nanocarrier formation or by forming the nanospheres in solutions or suspensions of the product to be complexed.

Acceptable pharmaceutical carriers for therapeutic used are well known in the pharmaceutical field, and are described, for example, in Remington: The Science and Practice of Pharmacy, (Gennaro et al., 1995), the disclosures of which are incorporated herein in their entirety for all purposes. Such materials are non-toxic to recipients at the dosages and concentrations employed, and include diluents, solubilizers, lubricants, suspending agents, encapsulating materials, solvents, thickeners, dispersants, buffers such as phosphate, citrate, acetate and other organic acid salts, anti-oxidants such as ascorbic acid, preservatives, low molecular weight (less than about 10 residues) peptides such as polyarginine, proteins such as serum albumin, gelatin or immunoglobulins, hydrophilic polymers such as poly(vinylpyrrolindinone), amino acids such as glycine, glutamic acid, aspartic acid or arginine, monosaccharides, disaccharides, and other carbohydrates including cellulose or its derivatives, glucose, mannose or dextrines, chelating agents such as EDTA, sugar alcohols such as mannitol or sorbitol, counter-ions such as sodium and/or non-ionic surfactants such as Tween™, Pluronics®, or PEG.

### Example 7

### Prospective Example: Encapsulation Efficiency of Radiolabelled Oligonucleotide in Nanocarriers Comprised of Tyrosine Triblock Copolymers

This prospective example demonstrates the encapsulation of the radiolabeled oligonucleotide, Vitravene® within nanocarriers formed from the tyrosine triblock copolymers synthesized in Example 1, using a modified technique known in the art (Al-Jamal et al., 2005). Vitravene® is an FDA-approved oligonucleotide used to treat cytomeglavirus invention retinitis in AIDS patients. Oligonucleotides are an emerging new class of therapeutics consisting of short nucleic acid chains that work by interfering with the processing of genetic information. Typically, they are unmodified or chemically modified single-stranded DNA or RNA molecules. They are relatively short (i.e., 19-25 nucleotides) and hybridise to a unique sequence in the total pool of DNA or RNA targets present in cells. New technological advances in molecular biology have led to the identification of genes associated with major human diseases and to the determination of their genetic basis. And now, oligonucleotide technologies are providing a highly specific strategy for targeting a wide range of diseases at the genetic level. In this example, the encapsulation and retention efficiency of an oligonucleotide, a 21-nucleotide phasphorothioate-based product with the sequence 5′-G-C-G-T-T-T-G-C-T-C-T-T-C-T-T-C-T-T-G-C-G-3′ in nanocarriers is evaluated, and compared to the encapsulation and retention efficiency of the same oligonucleotide in a conventional liposome formulation prepared by the same technique.

[³²P]-Oligonucleotide is reconstituted in doubly deionized water to make a final concentration of 0.9 μCi/50 μL. A mixture of cold (i.e., 2 mg) and radiolabelled (i.e., 5.7 μg or 0.9 μCi) oligonucleotide is dissolved in 5 ml water—drug to lipid percentage is 10%—and injected into the nanocarrier complex and lipid solutions, prepared as mentioned above. The suspension is ultracentrifuged in a Sorvall CombiPlus ultracentrifuge (Sorvall, Dupont, USA) at 42,000 rpm for 1 hour at 4° C., and washed to remove any unentrapped/non-interacting oligonucleotide. The pellets are suspended in 1 ml water for encapsulation efficiency and release studies. Radioactivity is measured in 10 μg of pellets suspension and supernatant. The weight of entrapped oligonucleotide is calculated accordingly; 0.9 μCi is equivalent to approximately 2 mg oligonucleotide. The percentage entrapment is calculated as the number of mg of oligonucleotide entrapped in 100 mg of total encapsulation material (total mass of block copolymer, etc.). Entrapment studies are carried out in triplicate, with the results expressed as percentage±SD.

In vitro release of oligonucleotide from nanocarriers and the comparator DSPC:CHOL (1:1) liposome formulation is measured using a dialysis technique. One milliliter of oligo-containing nanocarrier or liposome suspension is pipetted into the dialysis tubing, where the tubing has a molecular weight cutoff of 3500 Da, and then the tubing is sealed. The dialysis tubing is placed in 250 ml of deionized water in a 300-ml conical flask with constant stirring at 25° C. At intervals over 48 hours, 1 ml samples are taken and replaced with water of the same temperature. Each 1 ml sample is then added to 4 ml Optiphase “Safe” scintillation cocktail for quantification (e.g., LS 6500 multipurpose scintillation counter, Beckman, USA). Release studies are carried out in triplicate, with the results expressed as percentage±SD.

### Prospective Experimental Results

The influence of the negatively charged oligonucleotide on the morphology of drug-loaded nanocarriers should be minimal. Neutral liposomes (i.e., hydrodynamic diameter 730±13.5 nm, polydispersity index 0.35, zeta potential—2 mV) are used as a comparator to avoid the complications of electrostatic interaction. The encapsulation efficiency in FIG. 10 illustrates that nanocarriers comprised of tyrosine triblock copolymers have considerably greater entrapment efficiencies, and retention of those materials, than conventional liposomes.

### Example 8

### Prospective Example: Exposure of Mammalian Cells and Calcein-Containing Nanocarriers [Comprised of Tyrosine Triblock Copolymers] and Mammalian Cells to Controlled Ultrasonic Energy, and Evaluation of Cell Viability and Intracellular Calcein Delivery

This prospective example demonstrates the encapsulation of calcein within nanocarriers comprised substantially of tyrosein triblock copolymers. Said nanocarrier is synthesized, formed, and characterized in Examples 1 and 6. This is followed by disruption of the nanocarriers, calcein release, permeation of cellular membranes, and intracellular calcein delivery mediated by controlled ultrasonic energy. Calcein (623 Da, radius=0.6 nm), also known as fluorexon, fluorescein complex, is a fluorescent dye with an excitation and emission wavelengths of 495/515 nm, respectively. The acetomethoxy derivative of calcein (calcein AM) is used in biology, and in this experimental protocol, as it can be transported through the cellular membrane into live cells, which makes it useful for testing of cell viability and for short-term labeling of cells.

### Perspective Experimental Methods

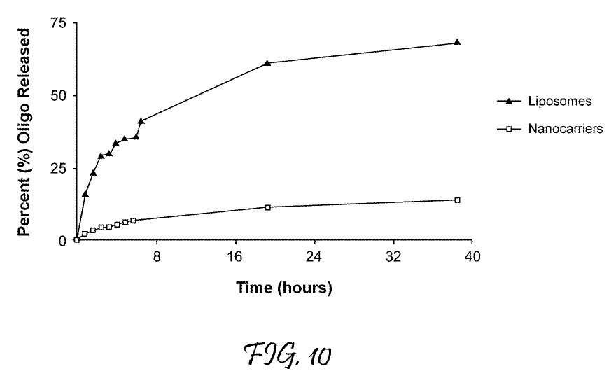

Ultrasound. Ultrasonic energy is produced using an immersible, focused, piezoceramic transducer. In this experimental system, two different matching resistance networks are necessary, allowing production of sound at 1.0 MHz and 3.0 MHz, similar to a method known in the art (Guzmán et al., 2001a, 2001b, 2002, and 2003; and Schlicher et al., 2006). A sinusoidal waveform is produced by, for example, programmable waveform generators (Stanford Research Instruments, Sunnyvale, Calif.) used in conjunction to control pulse length, frequency, and peak-to-peak voltage. The sinusoidal waveform is amplified by an RF broadband power amplifier (Electronic Navigation Industries, Rochester, N.Y.) before passing through a matching network and controlling the response of the transducer. The transducer is housed in a polycarbonate tank (FIG. 11 [601])—34.5×32×40 cm; containing approximately 34 liters of deionized, distilled, and partially degassed water at room temperature (i.e., 22° C. to 23° C.). A thick acoustic absorber is mounted opposite the transducer to minimize standing-wave formation (not illustrated). A three-axis micropositioning system (10 μm resolution; Velmex, Bloomfield, N.Y.) is mounted on top of the tank [603] to position samples and a hydrophone at desired locations in the tank. A PVDF membrane hydrophone (NTR Systems, Seattle, Wash.) is used to measure spatial-peak-temporal-peak negative pressure and to map and calibrate the acoustic field produced by the transducer versus the peak-to-peak voltage signal provided by a function generator.

Cell Culture. Cell culture and preparation is performed by a previously published procedure (Guzman et al., 2001). Briefly, Henrietta Lacks (HeLa) cells are cultured as monolayers in a humidified atmosphere of 95% air and 5% CO₂ at 37° C. in RMPI-1640 medium, supplemented with 100 μg/ml penicillin-streptomycin and 10% (i.e., v/v) heat-inactivated fetal bovine serum. Human aortic smooth muscle cells (AoSMC) are initiated from a cryopreserved stock and harvested at passage seven before each experiment. The cells are cultured as monolayers in a humidified atmosphere of 95% air and 5% CO₂ at 37° C. in MCDB-131 medium, supplemented with 100 ixg/ml penicillin-streptomycin and 10% (i.e., v/v) heat-inactivated fetal bovine serum. Both cell types are harvested by trypsin/EDTA digestion, washed, and re-suspended in pure RPMI for HeLa cells, and pure MCDB-131 for AoSMC cells.

Preparation of Nanocarriers (comprised of Tyrosine Triblock Copolymers) and Samples. Before ultrasound exposure, nanocarriers are prepared encapsulating calcein (i.e., 15 μM initial solution) by using the procedures of Example 6. Samples are prepared at a cell concentration of 10⁶ cells/ml, nanocarriers containing calcein, and the ultrasound contrast agent Optison® at concentrations of 0.30% v/v (˜1.6×10⁶ bubble/ml), 1.8% v/v (˜1.1×10⁷ bubble/ml), and 15.0% v/v (˜9.3×10⁷ bubble/ml). Optison® is an ultrasound contrast agent, a suspension of perfluorcarbon gas bubbles stabilized with denatured human albumin that is used to serve primarily as nuclei to promote acoustic cavitation activity in sonicated samples. The product name “Optison®” may hereafter be referred to as “contrast agent” or “ultrasound contrast agent.”

Before every experiment, the desired placement of the nanocarriers and cell samples in the acoustic field is found using the PVDF membrane hydrophone (FIG. 11 [604]). This location is approximately 1 cm and 0.5 cm out of the ultrasound's focus toward the transducer (601) (for 1.1 MHz and 3.1 MHz, respectively). The acoustic pressure is calibrated versus the peak-to-peak voltage of the signal created by the function generator, using the PVDF membrane hydrophone (604) at the desired location. These out-of-focus locations have a broader acoustic beam than at the focus, approximately 10.4-mm and 2.4-mm wide at half-amplitude (i.e., −6 dB) for 1.1 MHz and 3.1 MHz, respectively. This broader acoustic energy wave is more favorable for the experimental system shown in FIG. 11, allowing a more uniform acoustic exposure across the sample chamber and reducing “dead zones,” where cells or contrast agents are not uniformly exposed to said acoustic energy (Schlicher et al., 2006). In addition to the broad exposure zone, vigorous mixing, probably caused by microstreaming and acoustic cavitation during ultrasound exposure, further enables a more uniform exposure of all nanocarriers and mammalian cells in the sample chamber.

Samples are placed within chambers (605) constructed from a cylindrical bulb of polyethylene with an approximate dimension of 1.4 cm in height and 0.6 cm in diameter. Sample solutions are slowly aliquoted into the sample chamber (605) with a syringe, making sure to fill the chamber completely, however, without the production of air bubbles. A metal rod (606) is immediately inserted into the open end of the sample chamber and then attached to the three-axis positioning system (603), placing the sample in the desired location as determined by the hydrophone (604). After sample placement, a computer program is initiated to record hydrophone (604) output, and the exposure is initiated by triggering the function generator with the desired setting.

Exposures are performed at a burst length of 1 ms, 1% duty cycle (i.e., 10 pulses per second); pressures of 0.5, 0.8, 1.2, 1.7 and 2 MPa; total exposure times of 2, 10, 20, 100, 200, 1000 and 2000 ms; and frequencies of 1.1 MHz and 3.1 MHz. Total exposure time is simply the amount of time a sample is exposed to ultrasound, calculated by multiplying the number of ultrasound bursts times the burst length. After ultrasound exposure, samples are immediately transferred into 1.5-ml microcentrifuge tubes and allowed to “rest” for 5 minutes at room temperature. Samples in microcentrifuge tubes are then placed on ice and allowed to incubate until all samples have been exposed (1 hour to 2 hours).

After all nanocarrier samples and cell suspensions have been exposed, sonicated nanocarriers and cells are washed with phosphate buffered saline (PBS) and centrifuged (i.e., 900×g for 3 minutes) three times to remove non-ruptured nanocarriers, nanocarrier components, and extracellular calcein in the sample supernatant. The subsequent cell pellets are resuspended in a final volume of PBS containing propidium iodide (i.e., 2 mg/ml), a viability marker that stains nonviable cells with red fluorescence.

Quantitation of Cell Viability and Permeability. Flow cytometry is used to assay cell viability and permeability, a method of measuring the number of cells in a sample, the percentage of live cells in a sample, and certain characteristics of cells. These characteristics include size, shape, and in some of the experiments described herein, the rupture of nanocarriers encapsulating specific markers, the number of cells taking up said marker following exposure to ultrasound in the presence of ultrasound contrast agents. The cells are stained with a light-sensitive dye, placed in a fluid, and passed in a stream before a laser or other type of light. The measurements are based on how the light-sensitive dye reacts to the light. Specifically, in this experimental procedure, this measurement represents the fraction of cells containing intracellular calcein, where the loss of cell viability is determined by detecting the fluorescence intensity from calcein and propidium iodide, respectively, on a cell-by-cell basis. The viability of a sonicated sample compared with a “sham” sonicated (i.e., control) cell population is determined by accounting for lysed cells, intact dead cells, and viable cells. Viable cells are counted in each sample, normalized based on the fluid volume analyzed by the flow cytometer, and then normalized to the viability of a control sample. The analysis time of a particular sample in the flow cytometer is used as a measure of the sample volume analyzed, since the flow cytometer is operated at a constant flow rate.

### Prospective Experimental Results

Dependence of Cell Permeability and Viability on Ultrasound and Experimental Parameters. The influence of a variety of ultrasound and experimental parameters on cell viability, cell membrane permeability, nanocarrier disruption and calcein release, as well as intracellular calcein delivery can be determined with the experimental system illustrated in FIG. 1. Only some of the parameters that may be evaluated in this simple system include (1) the influence of ultrasonic pressure and frequency, (2) ultrasonic exposure time, (3) ultrasound contrast agent concentration, and (4) characteristics of the biological system (i.e., cell type) where extracellular and intracellular drug delivery is sought.

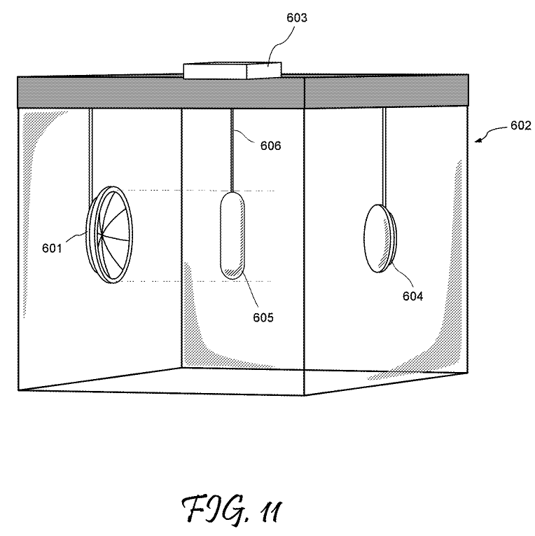

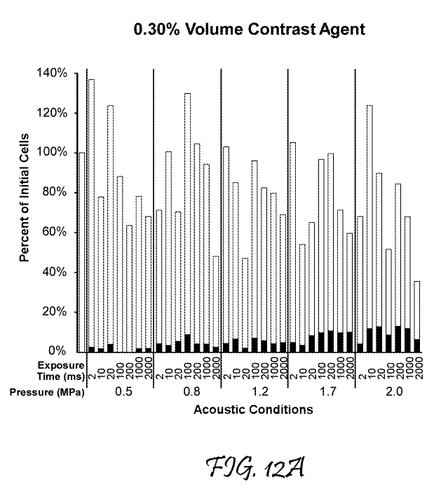

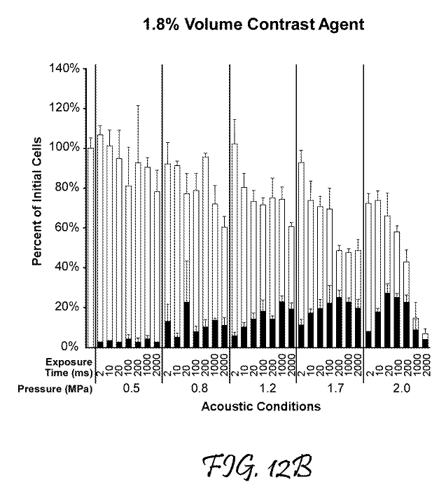

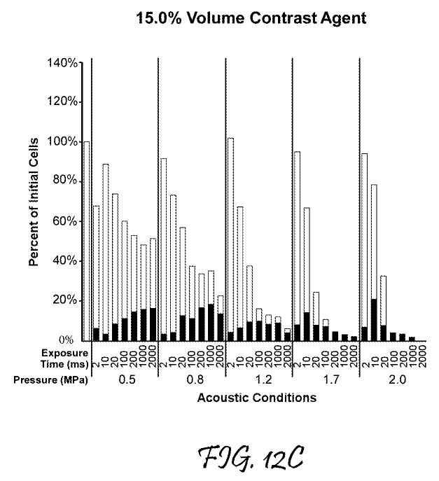

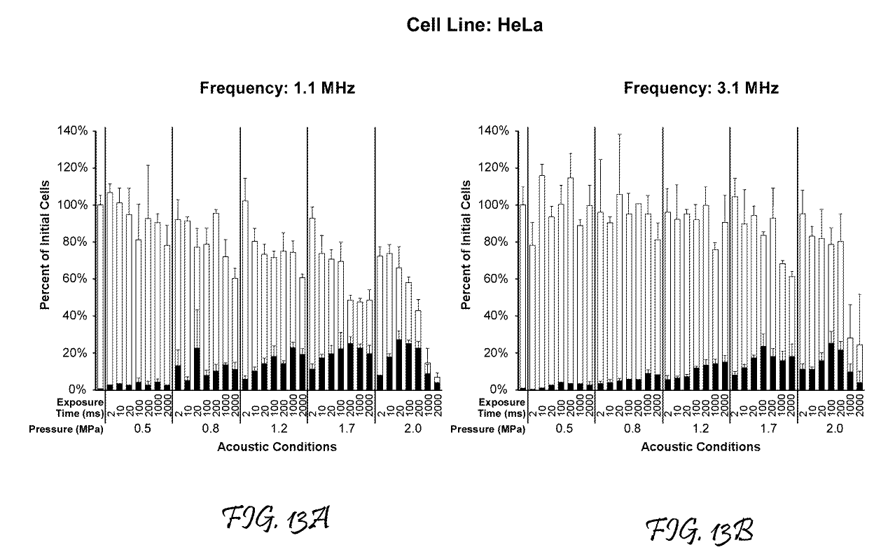

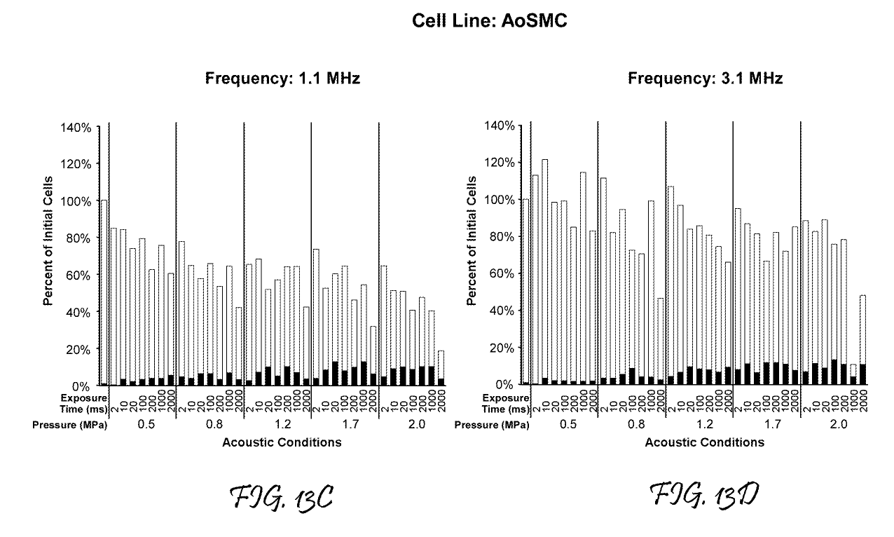

FIGS. 12A-12C illustrates the effect of changing the concentration of contrast agent (i.e., nucleation sites for cavitation) over a range of pressures and exposure times. Further, the effects of frequency and cell type are shown in FIGS. 13A-13D, wherein two different cell types, Henrietta Lacks (HeLa, FIGS. 13A-13B) and aortic smooth muscle cells (AoSMC; FIGS. 13C-13D), are sonicated at two different frequencies, 1.1 MHz and 3.1 MHz, over the same range of pressures and exposure times. In FIGS. 12A-12C, the total height of each bar represents the percent of cells remaining viable after sonication, which is subdivided into a black bar, representing the percent of viable cells with calcein uptake. All samples are normalized to the control sample (i.e., “sham” sonication), taken to represent 100% viability and 0% uptake.

In FIGS. 12A-12C and FIGS. 13A-13D, independently increasing pressure or exposure time increases the fraction of cells affected by insonation, increasing both the fraction of cells with uptake and the fraction of cells killed. The interaction of pressure and exposure time also has a statistically synergistic effect. Increasing contrast agent concentration (FIGS. 12A-12C) and decreasing frequency (FIGS. 13A-13B) also increases the effects on biological materials, by increasing uptake and cell death. In general, greater uptake and lower levels of death are seen in HeLa cells compared with AoSMC cells (FIGS. 13C-13D).

As with the present invention, a variety of conditions and parameters can be altered and evaluated that cumulatively result in mild to profound effects on nanocarrier rupture, calcein release, and on the alteration of insonated biological materials. These include (1) mild conditions that cause low levels of uptake calcein uptake, and almost no cell death, (2) moderate conditions that cause uptake into as many as one-third of cells and some cell death, and (3) strong conditions that kill almost all insonated cells.

While not wishing to be bound by any particular theory, the studies conducted in this relatively simple experimental system (FIG. 11) do have limitations. For example, the cell sample is exposed to a nonuniform acoustic field in said system. Acoustic scattering in the direction of the ultrasound beam by high concentrations of contrast agent can cause significant attenuation, which can approach 100% during the initial bursts of ultrasound (data not shown). Given these nonuniformities, only a fraction of the sample volume is exposed to a pressure above the threshold to, for example, inertial cavitation. Therefore, the size of any “cavitation zone” is expected to depend primarily on ultrasound pressure, frequency, and contrast agent concentration.

A far better testing environment would be to apply similar methods and techniques, but have nanocarrier and cells or tissue samples [FIG. 11 (606)] suspended inside one of several different commercially available ultrasound phantoms. An especially valuable apparatus is the CIRS series of ultrasound phantoms manufactured by CIRS, Tissue Simulation and Phantom Technology (Norfolk, Va.). Unlike human subjects or random scannable materials, this device offers a reliable medium which contains specific, known test objects for repeatable quantitative assessment of ultrasound performance over time. The phantom is constructed from the patented solid elastic material, Zerdine (see U.S. Pat. No. 5,196,343, the disclosures of which are hereby incorporated herein by reference in their entirety for all purposes). Zerdine, unlike other phantom materials on the market, is not affected by changes in temperature, can be subjected to boiling or freezing conditions without sustaining significant damage, and should be suitable for testing potential therapeutic applications of HIFU. At normal or room temperatures, the Zerdine material found in the Model 040 accurately simulates the ultrasound characteristics found in human tissue. Thus, this type of phantom should be ideal for use with a similar, but modified type of experimental system with characteristics similar to the experimental system illustrated in FIG. 11.

While a variety of methods and systems for use in acoustically mediated drug delivery have been described with reference to specific embodiments, it will be understood by those skilled in the art that methods and systems may be used by the present invention and that various, sometimes significant changes may be made and equivalents may be substituted for elements thereof without departing from the true spirit and scope of the invention. In addition, modifications may be made without departing from the essential teachings of the invention.

I claim:

1. A method suitable for the controlled intracellular and extracellular delivery of one or more therapeutic compounds to a region of a patient, the method comprising the acts (steps) of
(a) administering to said patient a therapeutic delivery system comprising a nanocarrier, in combination with one or more therapeutic compounds, wherein said nanocarrier is comprised of a biodegradable triblock copolymers or mixtures thereof, wherein said biodegradable triblock copolymers may be the same as or different from one another;
(b) administering to said patient one or more contrast agents, wherein said contrast agents may be the same as or different from one another, where steps (a) and (b) are performed
(i) in any order; or
(ii) simultaneously;
(c) applying therapeutic ultrasound to said region to induce rupturing of said nanocarrier, and disruption of cellular membranes and other structures of said patient, in said region, wherein said therapeutic compounds are encapsulated or embedded in said nanocarrier, thereby releasing one or more therapeutic compounds in said region, where said therapeutic ultrasound is applied at a level below the threshold level for lethal sonolysis or cytotoxicity; and
(d) allowing said therapeutic compounds to traverse said disrupted cellular membranes and/or other internal structures of said patient, in said region; and
(e) possibly repeating steps (a) through (d), in whole or in part, either independently or in any combination, one or more times.

2. The method as defined in claim 1, wherein at least one targeting moiety is associated with said nanocarrier.

3. The method as defined in claim 1, wherein at least one targeting moiety is associated with at least one of said contrast agents.

4. The method as defined in claim 1, wherein said nanocarrier is comprised substantially of a stabilized biodegradable triblock copolymers or mixtures thereof, wherein said stabilized biodegradable triblock copolymers may be the same as or different from one another.

5. The nanocarrier according to claim 1, further comprising at least one lipid, phospholipid, steroid, cholesterol, single-chain alcohol, polymer, copolymer, or surfactant.

6. A nanocarrier according to claim 1 wherein said triblock copolymer has an A-B-A structure
wherein each A end block is water-soluble, hydrophilic, and non-toxic;
and wherein the B middle block of said triblock copolymer is a polycarbonate with the same as or different repeating units with the following structure 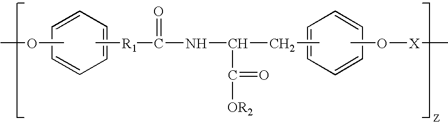
wherein X is: 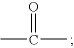
and wherein Z is between 2 and about 100, inclusive;
and wherein R₁ is CH═CH or (CH₂)ₙ, wherein n is from 0 to 18, inclusive;
and wherein R₂ is selected from the group consisting of hydrogen and straight and branched alkyl and alkylaryl groups containing up to 18 carbon atoms;

7. A nanocarrier according to claim 6 wherein said A end blocks of said triblock copolymer are poly(alkylene oxides) having the structure 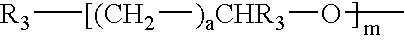
wherein m for each of said A end block is independently selected to provide a molecular weight for each of said A end block between about 1,000 and about 15,000 gm/mole;
and wherein R₃ for each of said A end block and within each of said A end block is independently selected from the group consisting of hydrogen and lower alkyl groups containing from one to four carbon atoms;
and wherein a is an integer greater than or equal to one.

8. A nanocarrier according to claim 6 wherein the end blocks of said triblock copolymer have the structure

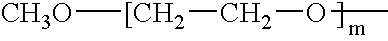

9. A nanocarrier according to claim 6 wherein Z of said triblock copolymer is about 10.

10. A nanocarrier according to claim 6 wherein R₁ and R₂, of said triblock copolymer contain an ether linkage.

11. A nanocarrier according to claim 6 wherein R₁ of said triblock copolymer is —CH₂—CH₂—.

12. A nanocarrier according to claim 6 wherein R₂ of said triblock copolymer is selected from the group consisting of ethyl, butyl, hexyl, octyl, decyl, dodecyl, and benzyl groups.

13. A nanocarrier according to claim 1 wherein said triblock copolymer has an A-B-A structure
wherein each A end block is water-soluble, hydrophilic, and non-toxic;
and wherein each B middle block is hydrophobic with the same as or different repeating units having the following structure 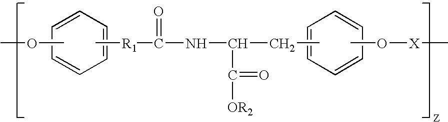
wherein X is: 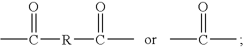
and wherein X is Z and is between 2 and about 100, inclusive;
and wherein R₁ is CH═CH or (CH₂)ₙ, wherein n is from 0 to 18, inclusive;
and wherein R₂ is selected from the group consisting of hydrogen and straight and branched alkyl and alkylaryl groups containing up to 18 carbon atoms;
and wherein R₂ is selected from the group consisting of (a) a bond or straight and branched alkyl and alkylaryl groups containing up to 18 carbon atoms; and (b) a hydrophobic compound complexed by said nanocarriers.

14. A nanocarrier according to claim 13 wherein the end groups of said triblock copolymer are poly(alkylene oxides) having the structure 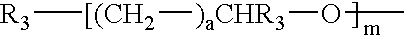
wherein m for each A is independently selected to provide a molecular weight for each A between about 1,000 and about 15,000 gm/mole;
and wherein R₃ for each A and within each A is independently selected from the group consisting of hydrogen and lower alkyl groups containing from one to four carbon atoms;
and wherein a is an integer greater than or equal to one.

15. A nanocarrier according to claim 13, wherein the A end blocks of said triblock copolymer have the structure

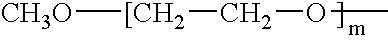

16. A nanocarrier according to claim 13 wherein Z of said triblock copolymer is about 10.

17. A nanocarrier according to claim 13 wherein one or more of R, R₁, and R₂, of said triblock copolymer contains an ether linkage.

18. A nanocarrier according to claim 13 wherein R₁ of said triblock copolymer is —CH₂—CH₂—.

19. A nanocarrier according to claim 13 wherein R of said triblock copolymer contains up to 12 carbon atoms.

20. A nanocarrier according to claim 19, wherein R of said triblock copolymer is selected from the group consisting of: —CH₂—CH₂—C(═O)—, —CH═CH—, —CH₂—CH(—OH)—, —CH₂-—C(═O)— and (—CH₂—)_{z}, wherein z is between 0 and 12, inclusive.

21. A nanocarrier according to claim 13, wherein R₂ of said triblock copolymer is selected from the group consisting of ethyl, butyl, hexyl, octyl, and benzyl groups.

22. The method as defined in claim 1, wherein said therapeutic compound is genetic material.

23. The therapeutic compound according to claim 22, wherein said genetic material comprises a nucleic acid, RNA or DNA of either natural or synthetic origin, comprising recombinant RNA and DNA, antisense RNA, RNA interference (RNAi), small interfering RNA (siRNA), or any combination thereof.

24. The nanocarrier according to claim 1, wherein said nanocarrier, which may be the same or different from one another, is embedded or dispersed in a drug delivery polymer matrix such as a hydrogel.

25. The method as defined in claim 1, wherein said method is for delivering one or more of said therapeutic compounds to the anterior or posterior portion of the eye.

26. The method as defined in claim 1, where previous to being administered to said patient, said therapeutic is embedded in a polymer, gel, or other matrix, allowing extended intracellular therapeutic release.

27. The method as defined in claim 1, wherein said therapeutic ultrasound comprises continuous wave ultrasound.

28. The method as defined in claim 1, wherein said therapeutic ultrasound is selected from the group consisting of amplitude and frequency modulated pulses.

29. The method as defined in claim 1, wherein said therapeutic ultrasound is applied externally to said patient.

30. The method as defined in claim 1, wherein said therapeutic ultrasound is applied endoscopically to said patient.

31. The method as defined in claim 1, wherein said nanocarrier is administered intravenously to said patient.
